SEQUENCE LISTING (81 sequences)

SEQ ID NO: 1 - DNA, 8017 bp, Thermothelomyces heterothallica
  source 1..8017: mol_type = genomic DNA
agggtaggtgggatgggcggggtgtagggtaggtcggtgtagggtaggtcggctgggcggggtgtagggtaggtcggttgggcggggtgtagggtaggtcggttgggatgggtgtagggtaggtcggccgggtgtagggtaggtcggctgggcggggtgtagggtaggtcggtgtagggtaggtgggatggggcgctatgtgcggccgcgagctcgcgagcccatttttagcgaaggccatacaaacgagttttgcggaacccgggattccacccccgaagccgccggcgcgtgcgccccgctgcgcatcggtcggtgggtatatgagaagggggcgggcaagccggaagccagaggcaactgctactgttagctgccgctggcctccgcggcccagggcgcggcacggctgcgttgaagtctcccagtctcccacccgttggctgcgcggatccgcccgtcttggtggttgcgagctcgcgagcccatttttagcgaaggccatacaaacgagttttgcggggcccgggattccaccccggaacccgccggcgcgtgcgccccgctgcgcatcggtcggtgggtatgtgagggaggaagaagaaaaaaaaaaaaagctcctgcgggggggctgtcgggcacgcctactttcgggcgacccggcacctctccgcggcagccttcgcaggccgctgttggtcccatttcatacgtcgccgccttcgcgtggtgccctacggtctgccggggtaccgacgattgcggcgagcaccgcctcagcaccgctgctgccaccggcgcgacctcgcccgggggtgcgcgcggcatctgggaagactctgcaggcgtaagggaataccccatgtgcgccgaggggtgggctatgtgggtgcttggcggttcgccagacctttctaaagccaccgggggtacctaccggttggggacgcctacagggctgaacctcccggtcgggcctcctcttggggcgcttaggcggcgacttcggggcgcgatcgctccccgctctcgcccgccgacggcgctctggggaattcaggaggggaaagcagatgtgacccgcggctcgaccggcgcattgccggacgagctgcgcggccacgcgggcccccgcgcccgccgacccagtaacttagtgaactcttccgccctgaaacacgggcggttggccctaaccggctcacgatagttacctggttgattctgccagtagtcatatgcttgtctcaaagattaagccatgcatgtctaagtataagcaattatacagcgaaactgcgaatggctcattaaatcagttatcgtttatttgatagtaccttactacatggataaccgtggtaattctagagctaatacatgctaaaaatcccgacttcggaagggatgtatttattagattaaaaaccaatgccctccggggctctctggtgattcatgataacttctcgaatcgcacggccttgcgccggcgatggttcattcaaatttctgccctatcaactttcgacggctgggtcttggccagccgtggtgacaacgggtaacggagggttagggctcgaccccggagaaggagcctgagaaacggctactacatccaaggaaggcagcaggcgcgcaaattacccaatcccgacacggggaggtagtgacaataaatactgatacagggctcttttgggtcttgtaattggaatgagtacaatttaaatcccttaacgaggaacaattggagggcaagtctggtgccagcagccgcggtaattccagctccaatagcgtatattaaagttgttgaggttaaaaagctcgtagttgaaccttgggcctagccggccggtccgcctcaccgcgtgcactggctcggctgggtctttccttctggagaaccgcatgcccttcactgggtgtgccggggaaccaggacttttactctgaacaaattagatcgcttaaagaaggcctatgctcgaatacattagcatggaataatagaataggacgtgtggttctattttgttggtttctaggaccgccgtaatgattaatagggacagtcgggggcatcagtattcaattgtcagaggtgaaattcttggatttattgaagactaactactgcgaaagcatttgccaaggatgttttcattaatcaggaacgaaagttaggggatcgaagacgatcagataccgtcgtagtcttaaccataaactatgccgattagggatcggacggcgttattttttgacccgttcggcaccttacgataaatcaaaatgtttgggctcctgggggagtatggtcgcaaggctgaaacttaaagaaattgacggaagggcaccaccaggggtggagcctgcggcttaatttgactcaacacggggaaactcaccaggtccagacacgatgaggattgacagattgagagctctttcttgatttcgtgggtggtggtgcatggccgttcttagttggtggagtgatttgtctgcttaattgcgataacgaacgagaccttaacctgctaaatagcccgtattgctttggcagtacgccggcttcttagagggactatcggctcaagccgatggaagtttgaggcaataacaggtctgtgatgcccttagatgttctgggccgcacgcgcgctacactgacagagccagcgagtactcccttggccggaaggcccgggtaatcttgttaaactctgtcgtgctggggatagagcattgcaattattgctcttcaacgaggaatccctagtaagcgcaagtcatcagcttgcgttgattacgtccctgccctttgtacacaccgcccgtcgctactaccgattgaatggctcagtgaggctttcggactggcccagagaggtcggcaacgaccactcagggccggaaagttatccaaactcggtcatttagaggaagtaaaagtcgtaacaaggtctccgttggtgaaccagcggagggatcattacagagctgcaaaactccctaaaccatcgtgaacgctacctagaccgttgcttcggcgggcggcgccctcgcgcgccccccctggggcccgcaccgcgggcgcccgccggaggtacaccaaactcttgatatgttatggccactctgagtctcctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagcatcctggcgggcatgcctgttcgagcgtcatttcaacccatcaagcccacggcttgtgttggggacctgcggctgcccgcaggccctgaaaaccagtggcgggctcgctagtcacaccgggcgtagtagcatacgacctcgctcagggcgtgctgcgggttccagccgtaaaacgaccttcacaacccaaggttgacctcggatcaggtaggaggacccgctgaacttaagcatatcaataagcggaggaaaagaaaccaacagggattgccctagtaacggcgagtgaagcggcaacagctcaaatttgaaatctggcttcggcccgagttgtaatttgcagaggaagctttaggcgcggcaccttctgagtcccctggaacggggcgccatagagggtgagagccccgtatagttggatgcctagcctgtgtaaagctccttcgacgagtcgagtagtttgggaatgctgctcaaaatgggaggtaaatttcttctaaagctaaataccggccagagaccgatagcgcacaagtagagtgatcgaaagatgaaaagcactttgaaaagagggttaaatagcacgtgaaattgttgaaagggaagcgcttgtgaccagacttgcgccgggctgatcatccggtgttctcaccggtgcactctgcccggctcaggccagcatcggttctcgcggggggataaaggcccggggaatgtagctcctccgggagtgttatagccccgggtgtaataccctcgcggggaccgaggttcgcgcatctgcaaggatgctggcgtaatggtcatcagcgacccgtcttgaaacacggaccaaggagtcaaggttttgcgcgagtgtttgggtgtaaaacccgcacgcgtaatgaaagtgaacgtaggtgagagcttcggcgcatcatcgaccgatcctgatgttttcggatggatttgagtaggagcgttaagccttggacccgaaagatggtgaactatgcttggatagggtgaagccagaggaaactctggtggaggctcgcagcggttctgacgtgcaaatcgatcgtcaaatctgagcatgggggcgaaagactaatcgaaccatctagtagctggttaccgccgaagtttccctcaggatagcagtgttgtcttcagttttatgaggtaaagcgaatgattagggactcgggggcgctttttagccttcatccattctcaaactttaaatatgtaagaagcccttgttacttagttgaacgtgggccttcgaatgtatcaacactagtgggccatttttggtaagcagaactggcgatgcgggatgaaccgaacgcggggttaaggtgccggagtggacgctcatcagacaccacaaaaggcgttagtacatcttgacagcaggacggtggccatggaagtcggaatccgctaaggactgtgtaacaactcacctgccgaatgtactagccctgaaaatggatggcgctcaagcgtcccacccataccccgccctcagggtagaaacgacgccctgaggagtaggcggccgtggaggtcagtgacgaagcctagggcgtgagcccgggtcgaacggcctctagtgcagatcttggtggtagtagcaaatacttcaatgagaacttgaaggaccgaagtggggaaaggttccatgtgaacagcggttggacatgggttagtcgatcctaagccatagggaagttccgtttcaaaggggcactcgtgccccgtgtggcgaaagggaagccggttaacattccggcacctggatgtgggttttgcgcggtaacgcaactgaacgcggagacgacggcgggggccccgggcagagttctcttttcttcttaacggtctatcaccctggaaacagtttgtctggagatagggtttaacggccggaagagcccgacacttctgtcgggtccggtgcgctctcgacgtcccttgaaaatccgcgggagggaataattctcacgccaggtcgtactcataaccgcagcaggtccccaaggtgaacagcctctggttgatagaacaatgtagataagggaagtcggcaaaatagatccgtaacttcgggataaggattggctctaagggttgggcacgttgggctttgggcggacgccctgggagcaggtcgcctctagccgggcaaccggcggggggcttccagcatccgggtgcagatgcccttagcaggcttcggccgtccggcgcgcggttaacaaccaacttagaactggtacggacagggggaatctgactgtctaattaaaacatagcattgcgatggccagaaagtggtgttgacgcaatgtgatttctgcccagtgctctgaatgtcaaagtgaagaaattcaaccaagcgcgggtaaacggcgggagtaactatgactctcttaaggtagccaaatgcctcgtcatctaattagtgacgcgcatgaatggattaacgagattcccactgtccctatctactatctagcgaaaccacagccaagggaacgggcttggcagaatcagcggggaaagaagaccctgttgagcttgactctagtttgacattgtgaaaagacataggaggtgtagaataggtgggagcttcggcgccggtgaaataccactactcctattgtttttttacttattcaatgaagcggggctggattttcgtccaacttctggttttaaggtccttcgcgggccgacccgggttgaagacattgtcaggtggggagtttggctggggcggcacatctgttaaaccataacgcaggtgtcctaaggggggctcatggagaacagaaatctccagtagaacaaaagggtaaaagtccccttgattttgattttcagtgtgaatacaaaccatgaaagtgtggcctatcgatcctttagtccctcgaaatttgaggctagaggtgccagaaaagttaccacagggataactggcttgtggcggccaagcgttcatagcgacgtcgctttttgatccttcgatgtcggctcttcctatcataccgaagcagaattcggtaagcgttggattgttcacccactaatagggaacgtgagctgggtttagaccgtcgtgagacaggttagttttaccctactgatgaactcatcgcaatggtaattcagcttagtacgagaggaaccgctgattcagataattggtttttgcggttgtccgaccgggcagtgccgcgaagctaccatctgctggataatggctgaacgcctctaagtcagaatccatgccagaacgcgatgatactacccgcacgttgtagacgtataagaataggctccggcctcgtatcctagcaggcgattcctccgccggcctcgaagttggccggcggtaattcgcgtattgcaatttcgacacgcgcgggatcaaatcctttgcagacgacttagatgtgcgaaagggtcctgtaagcagtagagtagccttgttgttacgatctgctgagggtaagccctccttcgcctagatttcccagcgagagcccgccggcggaacagccgggcgagccttacgggggaagccttaaggggattgagaagtggtgccgtgcgttcgcgcgcccctaggtcctttagccggccgcaggtgtagggtaggtcggttgggaggatggggtgtagggtaggtcggtgtagggtaggttggttgggaggatggggtgtagggtaggtcggccgggtgtagggtaggtcggtgtagggtaggtgggatggggcgctatatgcggccgcgagctcgcgagcctatttttagtgaaggctatataaataagctttacgttaccgggccttgctaccctcgagtggcgtgggccgtgctgcctactgggcattgctcgccgggctgtataagggaggggtcggggtcgcggtctagggtaggtcgggtgggatggggtgtagggtaggagaagcgctctagtcgtgtgtctttttctctaggtctattattagtactggctgtagggcgacgtgccctgccttgttataatattatattgtatgtttaggcctatactagcttgtaatctatttgtatctggcttattaggtacggcttcctttgtatataactagagaggctctggtatgcttcttagtatagcggtataggattcataatcatagtaatgataatcataatagtaataataataataatagtaatgataataataataatctatttatatcttatttaaaatgcttgtacggctgcctgctcttaaggagtagctagatatgagatggtagggtagctagctaacctaggctagacgttctcgtcccttagctatataagtgctatatattatagttagttatctaacctaccttcttacttgagcagaagaggtagggttctagtatagctagtagggcttctaggcctaagggcctgttattcgagttattataggttagtatttaatatagttatagggataggcctcgattacgggtataggataggtaggataggtatagggtaggtcggttaggaggatagggtgtaaggtaggtcggccgggtatagggtaggtagtaggttaggcggggtgtagggtaggtcggtgtagggtaggtgggatgggcggggtgtagggtaggtcggttgggaggatggggtgtagggtaggtcggtgtagggtaggtcgg

SEQ ID NO: 2 - protein, 195 aa, coronavirus (CoVs)
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGP

SEQ ID NO: 3 - DNA, 585 bp, coronavirus (CoVs)
  source 1..585: mol_type = other DNA
accaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcgagggcttcaactgctacttccccctccagtcctacggcttccagcccacctacggcgtcggctaccagccctaccgcgtcgtcgtcctctccttcgagctcctgcacgcccccgccaccgtctgcggccct

SEQ ID NO: 4 - protein, 195 aa, coronavirus (CoVs)
TNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGP

SEQ ID NO: 5 - DNA, 586 bp, coronavirus (CoVs)
  source 1..586: mol_type = other DNA
taccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaacatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcaagggcttcaactgctacttccccctccagtcctacggcttccagcccacctacggcgtcggctaccagccctaccgcgtcgtcgtcctctccttcgagctcctgcacgcccccgccaccgtctgcggccct

SEQ ID NO: 6 - protein, 195 aa, coronavirus (CoVs)
TNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGP

SEQ ID NO: 7 - DNA, 586 bp, coronavirus (CoVs)
  source 1..586: mol_type = other DNA
taccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaccatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcaagggcttcaactgctacttccccctccagtcctacggcttccagcccacctacggcgtcggctaccagccctaccgcgtcgtcgtcctctccttcgagctcctgcacgcccccgccaccgtctgcggccct

SEQ ID NO: 8 - protein, 892 aa, Thermothelomyces heterothallica
MKIWSGAALLGLAALATASHILPRDWEANDYYVLHLDADTSPQEVARSLGLSHEGPLGELRDHHVFVAKRAEHDVVKRELARRRKKRSLGLGGRDVLDGVLFSQKQRLRKPWEKRVVPRLFGPLPRRSVDEPVESLVQRQTEVARKLDIKDPIFHEQWHLFNTVQAGHDVNVTDVWLQGVTGKNATVAIVDDGLDMYSDDLRDNYYALGSYDFNDKADEPRPRLANDNHGTRCAGEVAAGRNNACGVGVAYDSNIAGLRILSKLISDADEAVALNYDFQHNQIYSCSWGPPDDGKSMDAPGILIRRAMLNAVQNGRGGLGSIYVFASGNGAHNEDNCNFDGYTNSIYSITVGALDRKGQHPYYSESCSAGLVVTYSSGSGDAIHTTDVGQNTCTSSHGGTSAAAPLAAGIFALVLQVRPDLSWRDMQYLAMDTAVPVNVDTGDYQDTTIGKKFSHTYGYGKLDSYAIVEAAKKWKKVKPQAWFYSPWIHVNQPIPQGDKGVVVEFEVTKEMLEEANLDRLEHVTVTMNVEHGRRGDLSVDLISPNKIVSHLSVTRKNDDSDKGYNDWTFMSVAHWGESGVGTWTIVVKDTEINQYTGKFIDWHLKLWGETRDASKAQLLPMPTEEDDDDHDVIATTTATAATTTVSKPEATGSVPADATDQPNRPVNSKPTDTSPAETSSSSSSSSSSAETDKTNTWLPSFLPTFGVSAATQAWIYGSLVLIVLFCAGLGIYLYLARRKRLRNKTRTDYEFELLDDDDDDDEEAAALTRGGGGGEKGVVGGGGGGGGKRGRRTRGGELYDAFAGESDEDSDDNDFAGGGGGGGGGGYRDRSDSRSRSRSDGSGSPIGISEKLPGRRDSLSGEEEHHVVGDDDDDDEDGTGNDQARPLQGGAR

SEQ ID NO: 9 - protein, 420 aa, Thermothelomyces heterothallica
MAGRLLLCLTAALSALGVSAAPAPDASGRPFIGVPVSNPGIANAIPNRYIVVYNNTFNDEDIDLHQSNVIKTIAKRNIAKRSLTGKLLSTTVNTYKINNWRAMALEADDATINEIFAAKEVSYIEQDAVISLNVRQMQSQATTGLARISHAQPGARTYIFDSSAGEGITAYVVDTGIRVTHEEFEGRATFAANFIDDVDTDEQGHGSHVAGTIGGKTFGVAKKVNLVAVKVLGADGSGSNSGVIAGMQFVASNATAMGLKGRAVMNMSLGGPASRAVNSAINQVEAAGVVPVVAAGNESQDTANTSPGSAEAAITVGAIDQTNDRMASFSNFGELVDIFAPGVNVQSVGIRSDTSTNTLSGTSMASPHVAGLAAYIMSLENITGVQAVSDRLKELAQATGARARGVPRGTTTLIANNGFA

SEQ ID NO: 10 - protein, 221 aa, coronavirus (CoVs)
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSEPEA

SEQ ID NO: 11 - DNA, 663 bp, coronavirus (CoVs)
  source 1..663: mol_type = other DNA
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcgagggcttcaactgctacttccccctccagtcctacggcttccagcccaccaacggcgtcggctaccagccctaccgcgtcgtcgtcctctccttcgagctcctgcacgcccccgccaccgtctgcggccctggcggcggcggcagcgagccggaggcc

SEQ ID NO: 12 - DNA, 14070 bp, synthetic construct
  source 1..14070: mol_type = other DNA
cgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcgagggcttcaactgctacttccccctccagtcctacggcttccagcccaccaacggcgtcggctaccagccctaccgcgtcgtcgtcctctccttcgagctcctgcacgcccccgccaccgtctgcggccctggcggcggcggcagcgagccggaggcctaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtgg

SEQ ID NO: 13 - DNA, 8756 bp, synthetic construct
  source 1..8756: mol_type = other DNA
ggcgcgccgtttaaactcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaaggagaggcgcgccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtatttaaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 14 - DNA, 11288 bp, synthetic construct
  source 1..11288: mol_type = other DNA
cgcgccgtttaaactcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaaggagaggcgcgccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtatttaaatgacgtgtatcataagttaagtaggtactgtacgttagtttgtttaccttcttcagccagtagtccgactttgctctcaagtgctcatccaaccctttggccttccaaatcttgataccgagagcctcaggcaggttggtatattctacctcaaatacctgccagatgttgaccaatgccgttgtcgttactccgtactacgcgaaccaatgccaacatgattctcctttcagtcacgagacgattcgagtgtcatggtagcagtactttggtagtaaagagtcactcaattgaacatgtcgtagctcattcgtaaccaagtcatgataaccctgaatttcaggggttcccttcagagacaggagccgtcatgttgcggcaagagaaaaaaaaatgaagaggaaacacacaaaaacgtgggaagaatagccatcagcggctaccctcatactccaactcctgcctgccttaggcctccggctcgctgccgccgccgccagggccgcagacggtggcgggggcgtgcaggagctcgaaggagaggacgacgacgcggtagggctggtagccgacgccgttggtgggctggaagccgtaggactggagggggaagtagcagttgaagccctcgacgccgttgcagggggtggagccggcctggtagatctcggtcgagatgtcgcgctcgaacggcttgaggttcgacttgcggaacaggcggtagaggtagttgtagttgccgccgaccttcgagtccaggttgttgctgttccaggcgatgacgcagccggtgaagtcgtcggggagcttgtagttgtagtcggcgatcttgccggtctggccgggggcgatctggcggacctcgtcgccgcggatgacgaaggagtcggcgtagacgttggtgaagcacaggtcgttgagcttggtggggctgacgccgtagcacttgaaggtggagaacgaggcgctgttgtacaggacgctgtagtcggcgacgcagttggagatgcgcttgcggttccaggcgtagacggaggcgaagcgggtggcgttgaagacctcgccgaacgggcagaggttggtagcagcggcgccagccacaagggcggcgagggtcgcgaacttggcgtacatggtggacgatggacgattgtctgtgtgagtaagggaaaggtggataggaggaaggggaaggaagttctgaggagggatggaagacggatgatgatgtgctagtaggccccaatgagccgaggagggaaggcctgatctcctttatatccaggaaccatcacggccgtttcccgccatgatctgcccccatctctcccgcgagtttcggaccgtcaacaccgatgaaaaaaagctgggcatcgcttgagagatccgggccctgtgttacgctgagtgcacagcgtgggcgatgtgggcagccggggcctcgacaaggatccaagaacaaccccccaccgcggggtaggttgaccctggcgaaggactggcaaacgaagggccgtgaatggccaacggtcagcaatccggggggctgttgatagtgggcaagggccttgcgcgttgcgatgcacgatattgcggagactgaggagccccactagcgcacccttgggccccgccaacccccaacgggccaaagggccagctctgggagcacggaaggagaacccgaaacacatgtttctggaaagagaaagcttgagcatggtatgatagtgtatttttttcctgcctttcttttctttcccaaccccaagttaggcaggtattcccgtgccttacatgggcacctgggagagactattttggcgcggggggggggtaaacctccgaattcgacctctccttccattgttgattgaattgaagaaggtcagctttcgcagttaccgcgccctcgaaaactcgccagaataatcgcgaaagtcgctccgagtcgctctctccaccttcgcatagtttccggatccacggcccgtgaaggacctgacgagcgctccggctgggtaaacctcggctgattctgggggtgaacaaaccaaaaacacgctggtcacgcgcaggggcgtcgttctagctcagcaaacgaaccacggcaccacgccgcccgcgccgagaggagcatctcgcaagccaggctttctagccaaggcacggggtatccatctctctgaggcgagacgagacgcatccgggccaaggagcactttagcgatgcgcgccctctctggcaaaactccggattttgctttcttgccgtggctaaacctcggtcgctctggcgattcacgtcccttcgctcccaggaaaccgaaccgtgcggccgactctgaacgagccgggcggctctcttcttcttttctcttctttcttctcgtcccatacgcttacttggaacactatgggcgacggcaactcgggctggctgttccggagcatggacatcgccgctctgcttatcgagtgtttggtgtgtcagacccgctccatcattccgtgaccgtggcaacccgggaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggg

SEQ ID NO: 15 - protein, 212 aa, unidentified
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 16 - DNA, 639 bp, unidentified
  source 1..639: mol_type = other DNA
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcgagggcttcaactgctacttccccctccagtcctacggcttccagcccaccaacggcgtcggctaccagccctaccgcgtcgtcgtcctctccttcgagctcctgcacgcccccgccaccgtctgcggcccttaa

SEQ ID NO: 17 - DNA, 14043 bp, synthetic construct
  source 1..14043: mol_type = other DNA
cgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcgagggcttcaactgctacttccccctccagtcctacggcttccagcccaccaacggcgtcggctaccagccctaccgcgtcgtcgtcctctccttcgagctcctgcacgcccccgccaccgtctgcggcccttaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtgg

SEQ ID NO: 18 - protein, 219 aa, unidentified
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPGSGEPEA

SEQ ID NO: 19 - DNA, 657 bp, synthetic construct
  source 1..657: mol_type = other DNA
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcgagggcttcaactgctacttccccctccagtcctacggcttccagcccacctacggcgtcggctaccagccctaccgcgtcgtcgtcctctccttcgagctcctgcacgcccccgccaccgtctgcggccctggcagcggcgagccggaggcc

SEQ ID NO: 20 - DNA, 14064 bp, synthetic construct
  source 1..14064: mol_type = other DNA
cgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcgagggcttcaactgctacttccccctccagtcctacggcttccagcccacctacggcgtcggctaccagccctaccgcgtcgtcgtcctctccttcgagctcctgcacgcccccgccaccgtctgcggccctggcagcggcgagccggaggcctaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtgg

SEQ ID NO: 21 - DNA, 11282 bp, synthetic construct
  source 1..11282: mol_type = other DNA
cgcgccgtttaaactcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaaggagaggcgcgccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtatttaaatgacgtgtatcataagttaagtaggtactgtacgttagtttgtttaccttcttcagccagtagtccgactttgctctcaagtgctcatccaaccctttggccttccaaatcttgataccgagagcctcaggcaggttggtatattctacctcaaatacctgccagatgttgaccaatgccgttgtcgttactccgtactacgcgaaccaatgccaacatgattctcctttcagtcacgagacgattcgagtgtcatggtagcagtactttggtagtaaagagtcactcaattgaacatgtcgtagctcattcgtaaccaagtcatgataaccctgaatttcaggggttcccttcagagacaggagccgtcatgttgcggcaagagaaaaaaaaatgaagaggaaacacacaaaaacgtgggaagaatagccatcagcggctaccctcatactccaactcctgcctgccttaggcctccggctcgccgctgccagggccgcagacggtggcgggggcgtgcaggagctcgaaggagaggacgacgacgcggtagggctggtagccgacgccgtaggtgggctggaagccgtaggactggagggggaagtagcagttgaagccctcgacgccgttgcagggggtggagccggcctggtagatctcggtcgagatgtcgcgctcgaacggcttgaggttcgacttgcggaacaggcggtagaggtagttgtagttgccgccgaccttcgagtccaggttgttgctgttccaggcgatgacgcagccggtgaagtcgtcggggagcttgtagttgtagtcggcgatcttgccggtctggccgggggcgatctggcggacctcgtcgccgcggatgacgaaggagtcggcgtagacgttggtgaagcacaggtcgttgagcttggtggggctgacgccgtagcacttgaaggtggagaacgaggcgctgttgtacaggacgctgtagtcggcgacgcagttggagatgcgcttgcggttccaggcgtagacggaggcgaagcgggtggcgttgaagacctcgccgaacgggcagaggttggtagcagcggcgccagccacaagggcggcgagggtcgcgaacttggcgtacatggtggacgatggacgattgtctgtgtgagtaagggaaaggtggataggaggaaggggaaggaagttctgaggagggatggaagacggatgatgatgtgctagtaggccccaatgagccgaggagggaaggcctgatctcctttatatccaggaaccatcacggccgtttcccgccatgatctgcccccatctctcccgcgagtttcggaccgtcaacaccgatgaaaaaaagctgggcatcgcttgagagatccgggccctgtgttacgctgagtgcacagcgtgggcgatgtgggcagccggggcctcgacaaggatccaagaacaaccccccaccgcggggtaggttgaccctggcgaaggactggcaaacgaagggccgtgaatggccaacggtcagcaatccggggggctgttgatagtgggcaagggccttgcgcgttgcgatgcacgatattgcggagactgaggagccccactagcgcacccttgggccccgccaacccccaacgggccaaagggccagctctgggagcacggaaggagaacccgaaacacatgtttctggaaagagaaagcttgagcatggtatgatagtgtatttttttcctgcctttcttttctttcccaaccccaagttaggcaggtattcccgtgccttacatgggcacctgggagagactattttggcgcggggggggggtaaacctccgaattcgacctctccttccattgttgattgaattgaagaaggtcagctttcgcagttaccgcgccctcgaaaactcgccagaataatcgcgaaagtcgctccgagtcgctctctccaccttcgcatagtttccggatccacggcccgtgaaggacctgacgagcgctccggctgggtaaacctcggctgattctgggggtgaacaaaccaaaaacacgctggtcacgcgcaggggcgtcgttctagctcagcaaacgaaccacggcaccacgccgcccgcgccgagaggagcatctcgcaagccaggctttctagccaaggcacggggtatccatctctctgaggcgagacgagacgcatccgggccaaggagcactttagcgatgcgcgccctctctggcaaaactccggattttgctttcttgccgtggctaaacctcggtcgctctggcgattcacgtcccttcgctcccaggaaaccgaaccgtgcggccgactctgaacgagccgggcggctctcttcttcttttctcttctttcttctcgtcccatacgcttacttggaacactatgggcgacggcaactcgggctggctgttccggagcatggacatcgccgctctgcttatcgagtgtttggtgtgtcagacccgctccatcattccgtgaccgtggcaacccgggaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggg

SEQ ID NO: 22 - protein, 219 aa, unidentified
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGSGEPEA

SEQ ID NO: 23 - DNA, 657 bp, synthetic construct
  source 1..657: mol_type = other DNA
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaacatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcaagggcttcaactgctacttccccctccagtcctacggcttccagcccacctacggcgtcggctaccagccctaccgcgtcgtcgtcctctccttcgagctcctgcacgcccccgccaccgtctgcggccctggcagcggcgagccggaggcc

SEQ ID NO: 24 - DNA, 14064 bp, synthetic construct
  source 1..14064: mol_type = other DNA
cgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaacatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcaagggcttcaactgctacttccccctccagtcctacggcttccagcccacctacggcgtcggctaccagccctaccgcgtcgtcgtcctctccttcgagctcctgcacgcccccgccaccgtctgcggccctggcagcggcgagccggaggcctaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtgg

SEQ ID NO: 25 - DNA, 11282 bp, synthetic construct
  source 1..11282: mol_type = other DNA
cgcgccgtttaaactcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaaggagaggcgcgccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtatttaaatgacgtgtatcataagttaagtaggtactgtacgttagtttgtttaccttcttcagccagtagtccgactttgctctcaagtgctcatccaaccctttggccttccaaatcttgataccgagagcctcaggcaggttggtatattctacctcaaatacctgccagatgttgaccaatgccgttgtcgttactccgtactacgcgaaccaatgccaacatgattctcctttcagtcacgagacgattcgagtgtcatggtagcagtactttggtagtaaagagtcactcaattgaacatgtcgtagctcattcgtaaccaagtcatgataaccctgaatttcaggggttcccttcagagacaggagccgtcatgttgcggcaagagaaaaaaaaatgaagaggaaacacacaaaaacgtgggaagaatagccatcagcggctaccctcatactccaactcctgcctgccttaggcctccggctcgccgctgccagggccgcagacggtggcgggggcgtgcaggagctcgaaggagaggacgacgacgcggtagggctggtagccgacgccgtaggtgggctggaagccgtaggactggagggggaagtagcagttgaagcccttgacgccgttgcagggggtggagccggcctggtagatctcggtcgagatgtcgcgctcgaacggcttgaggttcgacttgcggaacaggcggtagaggtagttgtagttgccgccgaccttcgagtccaggttgttgctgttccaggcgatgacgcagccggtgaagtcgtcggggagcttgtagttgtagtcggcgatgttgccggtctggccgggggcgatctggcggacctcgtcgccgcggatgacgaaggagtcggcgtagacgttggtgaagcacaggtcgttgagcttggtggggctgacgccgtagcacttgaaggtggagaacgaggcgctgttgtacaggacgctgtagtcggcgacgcagttggagatgcgcttgcggttccaggcgtagacggaggcgaagcgggtggcgttgaagacctcgccgaacgggcagaggttggtagcagcggcgccagccacaagggcggcgagggtcgcgaacttggcgtacatggtggacgatggacgattgtctgtgtgagtaagggaaaggtggataggaggaaggggaaggaagttctgaggagggatggaagacggatgatgatgtgctagtaggccccaatgagccgaggagggaaggcctgatctcctttatatccaggaaccatcacggccgtttcccgccatgatctgcccccatctctcccgcgagtttcggaccgtcaacaccgatgaaaaaaagctgggcatcgcttgagagatccgggccctgtgttacgctgagtgcacagcgtgggcgatgtgggcagccggggcctcgacaaggatccaagaacaaccccccaccgcggggtaggttgaccctggcgaaggactggcaaacgaagggccgtgaatggccaacggtcagcaatccggggggctgttgatagtgggcaagggccttgcgcgttgcgatgcacgatattgcggagactgaggagccccactagcgcacccttgggccccgccaacccccaacgggccaaagggccagctctgggagcacggaaggagaacccgaaacacatgtttctggaaagagaaagcttgagcatggtatgatagtgtatttttttcctgcctttcttttctttcccaaccccaagttaggcaggtattcccgtgccttacatgggcacctgggagagactattttggcgcggggggggggtaaacctccgaattcgacctctccttccattgttgattgaattgaagaaggtcagctttcgcagttaccgcgccctcgaaaactcgccagaataatcgcgaaagtcgctccgagtcgctctctccaccttcgcatagtttccggatccacggcccgtgaaggacctgacgagcgctccggctgggtaaacctcggctgattctgggggtgaacaaaccaaaaacacgctggtcacgcgcaggggcgtcgttctagctcagcaaacgaaccacggcaccacgccgcccgcgccgagaggagcatctcgcaagccaggctttctagccaaggcacggggtatccatctctctgaggcgagacgagacgcatccgggccaaggagcactttagcgatgcgcgccctctctggcaaaactccggattttgctttcttgccgtggctaaacctcggtcgctctggcgattcacgtcccttcgctcccaggaaaccgaaccgtgcggccgactctgaacgagccgggcggctctcttcttcttttctcttctttcttctcgtcccatacgcttacttggaacactatgggcgacggcaactcgggctggctgttccggagcatggacatcgccgctctgcttatcgagtgtttggtgtgtcagacccgctccatcattccgtgaccgtggcaacccgggaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggg

SEQ ID NO: 26 - protein, 219 aa, unidentified
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGSGEPEA

SEQ ID NO: 27 - DNA, 657 bp, synthetic construct
  source 1..657: mol_type = other DNA
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaccatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcaagggcttcaactgctacttccccctccagtcctacggcttccagcccacctacggcgtcggctaccagccctaccgcgtcgtcgtcctctccttcgagctcctgcacgcccccgccaccgtctgcggccctggcagcggcgagccggaggcc

SEQ ID NO: 28 - DNA, 14064 bp, synthetic construct
  source 1..14064: mol_type = other DNA
cgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaccatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcaagggcttcaactgctacttccccctccagtcctacggcttccagcccacctacggcgtcggctaccagccctaccgcgtcgtcgtcctctccttcgagctcctgcacgcccccgccaccgtctgcggccctggcagcggcgagccggaggcctaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtgg

SEQ ID NO: 29 - DNA, 11282 bp, synthetic construct
  source 1..11282: mol_type = other DNA
cgcgccgtttaaactcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaaggagaggcgcgccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtatttaaatgacgtgtatcataagttaagtaggtactgtacgttagtttgtttaccttcttcagccagtagtccgactttgctctcaagtgctcatccaaccctttggccttccaaatcttgataccgagagcctcaggcaggttggtatattctacctcaaatacctgccagatgttgaccaatgccgttgtcgttactccgtactacgcgaaccaatgccaacatgattctcctttcagtcacgagacgattcgagtgtcatggtagcagtactttggtagtaaagagtcactcaattgaacatgtcgtagctcattcgtaaccaagtcatgataaccctgaatttcaggggttcccttcagagacaggagccgtcatgttgcggcaagagaaaaaaaaatgaagaggaaacacacaaaaacgtgggaagaatagccatcagcggctaccctcatactccaactcctgcctgccttaggcctccggctcgccgctgccagggccgcagacggtggcgggggcgtgcaggagctcgaaggagaggacgacgacgcggtagggctggtagccgacgccgtaggtgggctggaagccgtaggactggagggggaagtagcagttgaagcccttgacgccgttgcagggggtggagccggcctggtagatctcggtcgagatgtcgcgctcgaacggcttgaggttcgacttgcggaacaggcggtagaggtagttgtagttgccgccgaccttcgagtccaggttgttgctgttccaggcgatgacgcagccggtgaagtcgtcggggagcttgtagttgtagtcggcgatggtgccggtctggccgggggcgatctggcggacctcgtcgccgcggatgacgaaggagtcggcgtagacgttggtgaagcacaggtcgttgagcttggtggggctgacgccgtagcacttgaaggtggagaacgaggcgctgttgtacaggacgctgtagtcggcgacgcagttggagatgcgcttgcggttccaggcgtagacggaggcgaagcgggtggcgttgaagacctcgccgaacgggcagaggttggtagcagcggcgccagccacaagggcggcgagggtcgcgaacttggcgtacatggtggacgatggacgattgtctgtgtgagtaagggaaaggtggataggaggaaggggaaggaagttctgaggagggatggaagacggatgatgatgtgctagtaggccccaatgagccgaggagggaaggcctgatctcctttatatccaggaaccatcacggccgtttcccgccatgatctgcccccatctctcccgcgagtttcggaccgtcaacaccgatgaaaaaaagctgggcatcgcttgagagatccgggccctgtgttacgctgagtgcacagcgtgggcgatgtgggcagccggggcctcgacaaggatccaagaacaaccccccaccgcggggtaggttgaccctggcgaaggactggcaaacgaagggccgtgaatggccaacggtcagcaatccggggggctgttgatagtgggcaagggccttgcgcgttgcgatgcacgatattgcggagactgaggagccccactagcgcacccttgggccccgccaacccccaacgggccaaagggccagctctgggagcacggaaggagaacccgaaacacatgtttctggaaagagaaagcttgagcatggtatgatagtgtatttttttcctgcctttcttttctttcccaaccccaagttaggcaggtattcccgtgccttacatgggcacctgggagagactattttggcgcggggggggggtaaacctccgaattcgacctctccttccattgttgattgaattgaagaaggtcagctttcgcagttaccgcgccctcgaaaactcgccagaataatcgcgaaagtcgctccgagtcgctctctccaccttcgcatagtttccggatccacggcccgtgaaggacctgacgagcgctccggctgggtaaacctcggctgattctgggggtgaacaaaccaaaaacacgctggtcacgcgcaggggcgtcgttctagctcagcaaacgaaccacggcaccacgccgcccgcgccgagaggagcatctcgcaagccaggctttctagccaaggcacggggtatccatctctctgaggcgagacgagacgcatccgggccaaggagcactttagcgatgcgcgccctctctggcaaaactccggattttgctttcttgccgtggctaaacctcggtcgctctggcgattcacgtcccttcgctcccaggaaaccgaaccgtgcggccgactctgaacgagccgggcggctctcttcttcttttctcttctttcttctcgtcccatacgcttacttggaacactatgggcgacggcaactcgggctggctgttccggagcatggacatcgccgctctgcttatcgagtgtttggtgtgtcagacccgctccatcattccgtgaccgtggcaacccgggaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggg

SEQ ID NO: 30 - DNA, 13415 bp, synthetic construct
  source 1..13415: mol_type = other DNA
gtttaaactgcgcaagcgcccttcatctcttcagccgagaatctgttgcctaattacatagtagatgctgggaacagtctgagaagcctgcgcaggttcttcattaattaccaagaacgcgggagaggtgggaagacgccgaaaactcgacgcggaccattcgctgttttccgatcttacaaaaaaaaggtatccgatttggggaacgtcgatgaaagtattgcaaaagtgacgagagttgcgcaactaactcgctgccgaagaagctgcggaagaaagagaacaccgaaagtggaataacgttacggatgtcctgacctcaaagttgaaaccagcccttcctgctctatttgggaaagcggcttgcccttgaatgcgctgcactgtggcacgactaccagtgatcgggaggagcaaactaccctggtccgttccttggtggggcggcactaggcccaacttagggtgatcggaggtcgatgccgcggtcctcgttggtctgggctcttctcatttcccggtttgcaccccccgttgcacctgctgatcgcccgccaacgccgatgaggttgcgcccagaccgacaatcaccgcggctgcattcccaagtatattgaagatggcaccaggtacccggttttgcgtcccagtcgtttggtgccaaatttgggagtttttgagcctcaagatctggggaaatcgacctcaacttccatacaagttaaagtcgcacacacggcgagttccacgaagagacacatttttttctgaaggcctctctccccgcacatcagaaaccaccaaataccaagactgcagaagccggggtaagtgggccaccgggactacactaaaatgcggggagaagcgagatccgttgcgaagggaagggatggggtgtgctgcggctttctccgctctcgtgcgccttttgcttgaatctagtgtacaccagggtaggctccgaaggagtatctacggcagcgctgttcgtgctgcgttgagagtcagggcggagacgagcaggcgacaggagcctcgcaccggcacttcggatcgcatttgcgcggagcgtcaaatacgctcttctgcggtcatcagagagcatcgtgaaccaaggttcttccgcagggcggcctgggcttcgcagagtcgcactcggcggacgccttccgtgtcacccctgataacctggctgccgcgcccagactcctccaatgaggtgtgtggttgccctcgccgacccttcagcaaccttaatcgcttccatcgcacggctccacgtcctcgaacgatgccctcagtccgtgcccggccgtggcaaccataacgtgacatcgccgcccagcctactagccgctatcgaccggttaggcttgtcaccgcagcgcccattctccatcgggcctctactctgatccacctcacccaccgcaagcactagcgagcctcaccagagtgcaagcgacacgacccgcttggcccttcgtccttgactatctcccagacctcttgccatcttgccgacgccgcccccttttttttctcctccccctgccggcaggtcggtggccccagtcccgagatggcattgctccgttgtccatgacgacccatcattcgatggctgactggcacactcgtcttgtttgagcatcgacggcccgcggcccgtctcccacggtacggaacctcgttgtacagtacctctcgtaatgatacccaacaccggggccgagcgctgggagggcggcgttcccgagaagccgggaaggcggctggccggctgacctttgtgacttggcgatggatgcggccatggagaatgtccgtccgaagcgacgcgacaattagcctggctaccatcgatataaattgggtgattcccagctcttgatgggcgtgtcttctgcctggcagccctcgtcttcagatcaagcaactgtgtgctgatcctcttccgcccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaccatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcaagggcttcaactgctacttccccctccagtcctacggcttccagcccacctacggcgtcggctaccagccctaccgcgtcgtcgtcctctccttcgagctcctgcacgcccccgccaccgtctgcggccctggcagcggcgagccggaggcctaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtcatttaaatcataaaccttttttttcacgtaacaaaatggcttcttttcagaccatgtgaaccatcttgatgccttgacctcttcagttctcactttaacgtagttcgcgtttgtctgtatgtcccagttgcatgtagttgagataaatacccctggaagtgggtctgggcctttgtgggacggagccctctttctgtggtctggagagcccgctctctaccgcctaccttcttaccacagtacactactcacacattgctgaactgacccatcataccgtactttatcctgttaattcgtggtgctgtcgactattctatttgctcaaatggagagcacattcatcggcgcagggatacacggtttatggaccccaagagtgtaaggactattattagtaatattatatgcctctaggcgccttaacttcaacaggcgagcactactaatcaacttttggtagacccaattacaaacgaccatacgtgccggaaattttgggattccgggcgcgcccgttggcggtaacagcaggtgccgttcgctgctgctcggtccttttcccgaaagtaccctgccttcctggcctcgacctccgcctgcggggctctgggcactgtcaaactttcgcagttttgcacccgcagcgattcgagcgggacccgcttcgccaacgcggatcaggagaggtaaaagtctcccctgtcctttctgccggagcatcctccgtccgtcgttcgctggttgttgtgtgtgtgtgtgtgtacagtactgtaggtacatagtgctcaccccagggcatttccccaatccgcgattgcgcataccataggcgtcatctgaatctgcgttacgtccggataacactacgcagtacgaaggagtcggtgctactacgcccgcaagttcaagtctacggtgtggctaaggtgctcaagggcatagttacacgcaccccgggcgagcttctcgcacctcgaccctttgccggggcgtcactggtccgacaactcataatccaccatcgcttgggcaggcctgcatcccccgtgggtcgcatccaagccgccggtaaataccggccgacacacacacacacacacgcaccctgtctagcgcgacgctacaaaagagaccggcaggcactccccgcgaccgcttgttctggccatcccgtctctcgcctctgcagttatcgcctgcctcatctctgcgtcctgataacttttgtcacctctgatccccccccgacgagacggtctcgccgaccaggacccgagatggcggaccgccatcctactctgtcccagtccttcgccgagcgggcgaagacggctagccacccgctcacccgctacctctttcggctcatggacctcaaggcctcgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagggcgacggcctcggccagcagtacaactcgcccagcaagttgatcaacatttgcggcaccgacattgtcatcgtagggcgtggcatcaccgccgccggcgacccgccctccgaggctgagaggtacaggagaaaagcctggaaggcctatctggcgcgtctggcgtgatttggggggagggggagaggagatgggggacgggaggggtcgccttggtcagtcttgtgcgtgtcctgcagcggattcgtcaccggggcagcacccaaaagagggagaaaaaggggaaaaaaaataaataaataaaaagggttaagttgttgaaaaaagtgttgtgagctctctggcaaggcgcgccacgaacctctctgaaggaggttctgagacacgcgcgattcttctgtatatagttttatttttcactctggagtgcttcgctccaccagtacataaaccttttttttcacgtaacaaaatggcttcttttcagaccatgtgaaccatcttgatgccttgacctcttcagttctcactttaacgtagttcgcgtttgtctgtatgtcccagttgcatgtagttgagataaatacccctggaagtgggtctgggcctttgtgggacggagccctctttctgtggtctggagagcccgctctctaccgcctaccttcttaccacagtacactactcacacattgctgaactgacccatcataccgtactttatcctgttaattcgtggtgctgtcgactattctatttgctcaaatggagagcacattcatcggcgcagggatacacggtttatggaccccaagagtgtaaggactattattagtaatattatatgcctctaggcgccttaacttcaacaggcgagcactactaatcaacttttggtagacccaattacaaacgaccatacgtgccggaaattttgggattccgtccgctctccccaaccaagctagaagaggcaacgaacagccaatcccggtgctaattaaattatatggttcattttttttaaaaaaattttttcttcccattttcctctcgcttttctttttcgcatcgtagttgatcaaagtccaagtcaagcgagctatttgtgcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 31 - protein, 212 aa, unidentified
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGP

SEQ ID NO: 32 - DNA, 639 bp, synthetic construct
  source 1..639: mol_type = other DNA
atgtacgccaagttcgcgaccctcgccgcccttgtggctggcgccgctgctaccaacctctgccccttcggcgaggtcttcaacgccacccgcttcgccagcgtctacgcctggaaccgcaagcggatcagcaactgcgtcgccgactacagcgtcctctacaactccgccagcttctcgaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctcgacagcaaggtcggcggcaactacaactaccggtaccgcctcttccgcaagtcgaacctgaagcccttcgagcgcgacatcagcaccgagatctaccaggccggcagcaagccgtgcaacggcgtcgagggcttcaactgctacttccccctccagagctacggcttccagcccaccaacggcgtcggctaccagccctaccgcgtcgtcgtcctcagcttcgagctcctgcacgcccccgccaccgtctgcggcccctaa

SEQ ID NO: 33 - protein, 219 aa, unidentified
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGSGEPEA

SEQ ID NO: 34 - DNA, 660 bp, synthetic construct
  source 1..660: mol_type = other DNA
atgtacgccaagttcgcgaccctcgccgcccttgtggctggcgccgctgctaccaacctctgccccttcggcgaggtcttcaacgccacccgcttcgccagcgtctacgcctggaaccgcaagcggatcagcaactgcgtcgccgactacagcgtcctctacaactccgccagcttctcgaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctcgacagcaaggtcggcggcaactacaactaccggtaccgcctcttccgcaagtcgaacctgaagcccttcgagcgcgacatcagcaccgagatctaccaggccggcagcaagccgtgcaacggcgtcgagggcttcaactgctacttccccctccagagctacggcttccagcccaccaacggcgtcggctaccagccctaccgcgtcgtcgtcctcagcttcgagctcctgcacgcccccgccaccgtctgcggccccggcagcggcgagccggaggcctaa

SEQ ID NO: 35 - DNA, 14043 bp, synthetic construct
  source 1..14043: mol_type = other DNA
gtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgcgaccctcgccgcccttgtggctggcgccgctgctaccaacctctgccccttcggcgaggtcttcaacgccacccgcttcgccagcgtctacgcctggaaccgcaagcggatcagcaactgcgtcgccgactacagcgtcctctacaactccgccagcttctcgaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctcgacagcaaggtcggcggcaactacaactaccggtaccgcctcttccgcaagtcgaacctgaagcccttcgagcgcgacatcagcaccgagatctaccaggccggcagcaagccgtgcaacggcgtcgagggcttcaactgctacttccccctccagagctacggcttccagcccaccaacggcgtcggctaccagccctaccgcgtcgtcgtcctcagcttcgagctcctgcacgcccccgccaccgtctgcggcccctaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtggcgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 36 - DNA, 14064 bp, synthetic construct
  source 1..14064: mol_type = other DNA
gtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgcgaccctcgccgcccttgtggctggcgccgctgctaccaacctctgccccttcggcgaggtcttcaacgccacccgcttcgccagcgtctacgcctggaaccgcaagcggatcagcaactgcgtcgccgactacagcgtcctctacaactccgccagcttctcgaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctcgacagcaaggtcggcggcaactacaactaccggtaccgcctcttccgcaagtcgaacctgaagcccttcgagcgcgacatcagcaccgagatctaccaggccggcagcaagccgtgcaacggcgtcgagggcttcaactgctacttccccctccagagctacggcttccagcccaccaacggcgtcggctaccagccctaccgcgtcgtcgtcctcagcttcgagctcctgcacgcccccgccaccgtctgcggccccggcagcggcgagccggaggcctaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtggcgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 37 - protein, 212 aa, unidentified
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGP

SEQ ID NO: 38 - DNA, 639 bp, synthetic construct
  source 1..639: mol_type = other DNA
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaacatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcaagggcttcaactgctacttccccctccagtcctacggcttccagcccacctacggcgtcggctaccagccctaccgcgtcgtcgtcctctccttcgagctcctgcacgcccccgccaccgtctgcggcccttaa

SEQ ID NO: 39 - DNA, 14043 bp, synthetic construct
  source 1..14043: mol_type = other DNA
gtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaacatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcaagggcttcaactgctacttccccctccagtcctacggcttccagcccacctacggcgtcggctaccagccctaccgcgtcgtcgtcctctccttcgagctcctgcacgcccccgccaccgtctgcggcccttaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtggcgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 40 - DNA, 20 bp, unidentified
  source 1..20: mol_type = other DNA
ctcgcaccgtaaggaccaag

SEQ ID NO: 41 - DNA, 18 bp, unidentified
  source 1..18: mol_type = other DNA
ccacgtaagcggaagttg

SEQ ID NO: 42 - DNA, 19 bp, unidentified
  source 1..19: mol_type = other DNA
cgttgaagacctcgccgaa

SEQ ID NO: 43 - DNA, 20 bp, unidentified
  source 1..20: mol_type = other DNA
agcgtgacccactcaggtaa

SEQ ID NO: 44 - DNA, 20 bp, unidentified
  source 1..20: mol_type = other DNA
agcgtgacccactcaggtaa

SEQ ID NO: 45 - DNA, 20 bp, unidentified
  source 1..20: mol_type = other DNA
cactacgtcaagttcgtcga

SEQ ID NO: 46 - DNA, 19 bp, unidentified
  source 1..19: mol_type = other DNA
gccgtagaagtcgttggag

SEQ ID NO: 47 - DNA, 19 bp, unidentified
  source 1..19: mol_type = other DNA
cggtcacgtagatcttggg

SEQ ID NO: 48 - DNA, 20 bp, unidentified
  source 1..20: mol_type = other DNA
aacctcgagacgctcttcta

SEQ ID NO: 49 - DNA, 19 bp, unidentified
  source 1..19: mol_type = other DNA
catcatccgtcttccatcc

SEQ ID NO: 50 - DNA, 20 bp, unidentified
  source 1..20: mol_type = other DNA
ggtaaacatgtccaggcttc

SEQ ID NO: 51 - DNA, 19 bp, unidentified
  source 1..19: mol_type = other DNA
cttcagagacaggagccgt

SEQ ID NO: 52 - DNA, 22 bp, unidentified
  source 1..22: mol_type = other DNA
aggaaggagacatcgctctttt

SEQ ID NO: 53 - DNA, 20 bp, unidentified
  source 1..20: mol_type = other DNA
agcctggaaggcctatctgg

SEQ ID NO: 54 - DNA, 20 bp, unidentified
  source 1..20: mol_type = other DNA
tagcgcgaatactgctgtgg

SEQ ID NO: 55 - DNA, 20 bp, unidentified
  source 1..20: mol_type = other DNA
cctacacttgcaccaagctg

SEQ ID NO: 56 - DNA, 20 bp, unidentified
  source 1..20: mol_type = other DNA
ggcacgtatggtcgtttgta

SEQ ID NO: 57 - DNA, 20 bp, unidentified
  source 1..20: mol_type = other DNA
ccctctacttcgtgtccatg

SEQ ID NO: 58 - DNA, 20 bp, unidentified
  source 1..20: mol_type = other DNA
cagttctctacgttggcctc

SEQ ID NO: 59 - protein, 219 aa, coronavirus (CoVs)
MYAKFATLAALVAGAAATNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPGSGEPEA

SEQ ID NO: 60 - DNA, 657 bp, coronavirus (CoVs)
  source 1..657: mol_type = genomic DNA
atgtacgccaagttcgcgaccctcgccgcccttgtggctggcgccgctgctaccaacctctgcccgttcgacgaggtcttcaacgccacccgcttcgccagcgtctacgcctggaaccgcaagcgcatctcgaactgcgtcgccgactactcggtcctgtacaacctcgcccccttcttcaccttcaagtgctacggcgtctcgcccaccaagctcaacgacctgtgcttcaccaacgtctacgccgactcgttcgtcatccgcggcgacgaggtccgccagatcgcccccggccagaccggcaacatcgccgactacaactacaagctgcccgacgacttcaccggctgcgtcatcgcctggaactcgaacaagctcgacagcaaggtctcgggcaactacaactacctctaccgcctgttccgcaagagcaacctgaagcccttcgagcgcgacatcagcaccgagatctaccaggccggcaacaagccctgcaacggcgtcgccggcttcaactgctacttccccctccgctcgtacagcttccgccccacctacggcgtcggccaccagccctaccgcgtcgtcgtcctcagcttcgagctgctccacgcccccgccaccgtctgcggccccggcagcggcgagccggaggcc

SEQ ID NO: 61 - DNA, 14064 bp, synthetic construct
  source 1..14064: mol_type = other DNA
cgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgcgaccctcgccgcccttgtggctggcgccgctgctaccaacctctgcccgttcgacgaggtcttcaacgccacccgcttcgccagcgtctacgcctggaaccgcaagcgcatctcgaactgcgtcgccgactactcggtcctgtacaacctcgcccccttcttcaccttcaagtgctacggcgtctcgcccaccaagctcaacgacctgtgcttcaccaacgtctacgccgactcgttcgtcatccgcggcgacgaggtccgccagatcgcccccggccagaccggcaacatcgccgactacaactacaagctgcccgacgacttcaccggctgcgtcatcgcctggaactcgaacaagctcgacagcaaggtctcgggcaactacaactacctctaccgcctgttccgcaagagcaacctgaagcccttcgagcgcgacatcagcaccgagatctaccaggccggcaacaagccctgcaacggcgtcgccggcttcaactgctacttccccctccgctcgtacagcttccgccccacctacggcgtcggccaccagccctaccgcgtcgtcgtcctcagcttcgagctgctccacgcccccgccaccgtctgcggccccggcagcggcgagccggaggcctaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtgg

SEQ ID NO: 62 - DNA, 11282 bp, synthetic construct
  source 1..11282: mol_type = other DNA
cgcgccgtttaaactcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaaggagaggcgcgccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtatttaaatgacgtgtatcataagttaagtaggtactgtacgttagtttgtttaccttcttcagccagtagtccgactttgctctcaagtgctcatccaaccctttggccttccaaatcttgataccgagagcctcaggcaggttggtatattctacctcaaatacctgccagatgttgaccaatgccgttgtcgttactccgtactacgcgaaccaatgccaacatgattctcctttcagtcacgagacgattcgagtgtcatggtagcagtactttggtagtaaagagtcactcaattgaacatgtcgtagctcattcgtaaccaagtcatgataaccctgaatttcaggggttcccttcagagacaggagccgtcatgttgcggcaagagaaaaaaaaatgaagaggaaacacacaaaaacgtgggaagaatagccatcagcggctaccctcatactccaactcctgcctgccttaggcctccggctcgccgctgccggggccgcagacggtggcgggggcgtggagcagctcgaagctgaggacgacgacgcggtagggctggtggccgacgccgtaggtggggcggaagctgtacgagcggagggggaagtagcagttgaagccggcgacgccgttgcagggcttgttgccggcctggtagatctcggtgctgatgtcgcgctcgaagggcttcaggttgctcttgcggaacaggcggtagaggtagttgtagttgcccgagaccttgctgtcgagcttgttcgagttccaggcgatgacgcagccggtgaagtcgtcgggcagcttgtagttgtagtcggcgatgttgccggtctggccgggggcgatctggcggacctcgtcgccgcggatgacgaacgagtcggcgtagacgttggtgaagcacaggtcgttgagcttggtgggcgagacgccgtagcacttgaaggtgaagaagggggcgaggttgtacaggaccgagtagtcggcgacgcagttcgagatgcgcttgcggttccaggcgtagacgctggcgaagcgggtggcgttgaagacctcgtcgaacgggcagaggttggtagcagcggcgccagccacaagggcggcgagggtcgcgaacttggcgtacatggtggacgatggacgattgtctgtgtgagtaagggaaaggtggataggaggaaggggaaggaagttctgaggagggatggaagacggatgatgatgtgctagtaggccccaatgagccgaggagggaaggcctgatctcctttatatccaggaaccatcacggccgtttcccgccatgatctgcccccatctctcccgcgagtttcggaccgtcaacaccgatgaaaaaaagctgggcatcgcttgagagatccgggccctgtgttacgctgagtgcacagcgtgggcgatgtgggcagccggggcctcgacaaggatccaagaacaaccccccaccgcggggtaggttgaccctggcgaaggactggcaaacgaagggccgtgaatggccaacggtcagcaatccggggggctgttgatagtgggcaagggccttgcgcgttgcgatgcacgatattgcggagactgaggagccccactagcgcacccttgggccccgccaacccccaacgggccaaagggccagctctgggagcacggaaggagaacccgaaacacatgtttctggaaagagaaagcttgagcatggtatgatagtgtatttttttcctgcctttcttttctttcccaaccccaagttaggcaggtattcccgtgccttacatgggcacctgggagagactattttggcgcggggggggggtaaacctccgaattcgacctctccttccattgttgattgaattgaagaaggtcagctttcgcagttaccgcgccctcgaaaactcgccagaataatcgcgaaagtcgctccgagtcgctctctccaccttcgcatagtttccggatccacggcccgtgaaggacctgacgagcgctccggctgggtaaacctcggctgattctgggggtgaacaaaccaaaaacacgctggtcacgcgcaggggcgtcgttctagctcagcaaacgaaccacggcaccacgccgcccgcgccgagaggagcatctcgcaagccaggctttctagccaaggcacggggtatccatctctctgaggcgagacgagacgcatccgggccaaggagcactttagcgatgcgcgccctctctggcaaaactccggattttgctttcttgccgtggctaaacctcggtcgctctggcgattcacgtcccttcgctcccaggaaaccgaaccgtgcggccgactctgaacgagccgggcggctctcttcttcttttctcttctttcttctcgtcccatacgcttacttggaacactatgggcgacggcaactcgggctggctgttccggagcatggacatcgccgctctgcttatcgagtgtttggtgtgtcagacccgctccatcattccgtgaccgtggcaacccgggaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggg

SEQ ID NO: 63 - protein, 219 aa, coronavirus (CoVs)
MYAKFATLAALVAGAAATNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPGSGEPEA

SEQ ID NO: 64 - DNA, 657 bp, coronavirus (CoVs)
  source 1..657: mol_type = genomic DNA
atgtacgccaagttcgcgaccctcgccgcccttgtggctggcgccgctgctaccaacctctgccccttcgacgaggtcttcaacgccacccgcttcgcctccgtctacgcctggaaccgcaagcgcatctccaactgcgtcgccgactacagcgtcctctacaacttcgccccgttcttcgccttcaagtgctacggcgtctcgcccaccaagctcaacgacctgtgcttcaccaacgtctacgccgactccttcgtcatccgcggcaacgaggtctcccagatcgccccgggccagaccggcaacatcgccgactacaactacaagctccccgacgacttcaccggctgcgtcatcgcctggaacagcaacaagctggactcgaaggtcggcggcaactacaactaccgctaccgcctcttccgcaagtcgaacctgaagccgttcgagcgcgacatctccaccgagatctaccaggccggcaacaagccgtgcaacggcgtggccggcgtcaactgctacttcccgctccagtcctacggcttccgccccacctacggcgtcggccaccagccctaccgcgtcgtcgtcctgagcttcgagctgctgcacgcccccgccaccgtctgcggccccggctccggcgagcccgaggcc

SEQ ID NO: 65 - DNA, 14064 bp, synthetic construct
  source 1..14064: mol_type = other DNA
cgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaaccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgcgaccctcgccgcccttgtggctggcgccgctgctaccaacctctgccccttcgacgaggtcttcaacgccacccgcttcgcctccgtctacgcctggaaccgcaagcgcatctccaactgcgtcgccgactacagcgtcctctacaacttcgccccgttcttcgccttcaagtgctacggcgtctcgcccaccaagctcaacgacctgtgcttcaccaacgtctacgccgactccttcgtcatccgcggcaacgaggtctcccagatcgccccgggccagaccggcaacatcgccgactacaactacaagctccccgacgacttcaccggctgcgtcatcgcctggaacagcaacaagctggactcgaaggtcggcggcaactacaactaccgctaccgcctcttccgcaagtcgaacctgaagccgttcgagcgcgacatctccaccgagatctaccaggccggcaacaagccgtgcaacggcgtggccggcgtcaactgctacttcccgctccagtcctacggcttccgccccacctacggcgtcggccaccagccctaccgcgtcgtcgtcctgagcttcgagctgctgcacgcccccgccaccgtctgcggccccggctccggcgagcccgaggcctaaggccctatgacctcccgagtcctggtcgagagaggggagctggagagagagagagagagagagagggagcatgaagccgtactgattcggcaaaccattttgaagcctggacatgtttaccttcgttttctcttcttggatggtttgcattattattagccgcttttcttttttctctccattcgttatacatattttttgttttgttttcctcttgttgttcattgtttggccctgggccttttttcttcttctcatttctcttttcacttcttcttcttcttcttcttcttcttcttcttcctcctcctcctcctccttcgctttgcgggaccgcttcttggggctcgggaggaccagtggtatgcttgcgagcacacacatcgatgcatggtgtttctttcatgttgtacctatcgttcagtgttttgcccccggtttgggcatttcattcgcttcgcttgtacatacacatggacaatacattcttcgtatggcttcaggctagacatctcccctaccaaccacacctgcaccatgcccgtcttgtcaacctacctaacttacataccgatgttcgccggccgcgagctgctgcagaggtttgtgacggagacacacatcgatttctcacctgcccgtgg

SEQ ID NO: 66 - DNA, 23 bp, unidentified
  source 1..23: mol_type = other DNA
agcttgtagttgtagtcggcgat

SEQ ID NO: 67 - RNA, 36 nt, unidentified
  source 1..36: mol_type = other RNA
ctacggcatgaaccactacagttttagagctatgct

SEQ ID NO: 68 - RNA, 36 nt, unidentified
  source 1..36: mol_type = other RNA
gggtccttgaacttacgagggttttagagctatgct

SEQ ID NO: 69 - RNA, 36 nt, unidentified
  source 1..36: mol_type = other RNA
cgaccccatgaatctcagcggttttagagctatgct

SEQ ID NO: 70 - RNA, 36 nt, unidentified
  source 1..36: mol_type = other RNA
catctcctcagcattcaccggttttagagctatgct

SEQ ID NO: 71 - RNA, 36 nt, unidentified
  source 1..36: mol_type = other RNA
aagcactatgagcaatgcgggttttagagctatgct

SEQ ID NO: 72 - RNA, 36 nt, unidentified
  source 1..36: mol_type = other RNA
gcacttggaccacgtcagcggttttagagctatgct

SEQ ID NO: 73 - RNA, 36 nt, unidentified
  source 1..36: mol_type = other RNA
ctcactggccaatcgaacaggttttagagctatgct

SEQ ID NO: 74 - RNA, 36 nt, unidentified
  source 1..36: mol_type = other RNA
cggctgtgctggccgccgcggttttagagctatgct

SEQ ID NO: 75 - RNA, 36 nt, unidentified
  source 1..36: mol_type = other RNA
cgcccattataccctccccagttttagagctatgct

SEQ ID NO: 76 - RNA, 36 nt, unidentified
  source 1..36: mol_type = other RNA
tgatgttgataagaatgccggttttagagctatgct

SEQ ID NO: 77 - RNA, 36 nt, unidentified
  source 1..36: mol_type = other RNA
gtaggtctgcgcaaggacgggttttagagctatgct

SEQ ID NO: 78 - RNA, 36 nt, unidentified
  source 1..36: mol_type = other RNA
ggtgagcgagactcagtacggttttagagctatgct

SEQ ID NO: 79 - RNA, 36 nt, unidentified
  source 1..36: mol_type = other RNA
gtactcgtcgaaccactggcgttttagagctatgct

SEQ ID NO: 80 - RNA, 36 nt, unidentified
  source 1..36: mol_type = other RNA
cgtggcggcgacaagaccacgttttagagctatgct

SEQ ID NO: 81 - protein, 17 aa, unidentified
MYAKFATLAALVAGAAA

(19) United States
(12) Patent Application Publication
(10) Pub. No.: US 2025/0042949 A1
(43) Pub. Date: Feb. 6, 2025

(54) EXPRESSION OF MULTIPLE IMMUNE-ACTIVE MOLECULES IN GENETICALLY-MODIFIED FILAMENTOUS FUNGI

(71) Applicant: DYADIC INTERNATIONAL (USA), INC., Jupiter, FL (US)

(72) Inventors: Ronen Tchelet, Budapest (HU); Mark Aaron Emalfarb, Jupiter, FL (US); Marika Vitikainen, Espoo (FI); Anne Huuskonen, Espoo (FI); Markku Saloheimo, Espoo (FI)

(21) Appl. No.: 18/709,413

(22) PCT Filed: Nov. 10, 2022
(86) PCT No.: PCT/IB2022/060813

§ 371 Date: May 10, 2024

Related U.S. Application Data
(60) Provisional application No. 63/278,112, filed on Nov. 11, 2021.

(51) Int. Cl. C07K 14/005; C12N 15/80

(52) U.S. Cl. CPC C07K 14/005; C12N 15/80; C12N 2770/20022; C12N 2770/20051

(57) ABSTRACT

The present invention relates to genetically modified ascomycetous filamentous fungi, particularly of the species Thermothelomyces heterothallica, capable of producing multiple immune-active molecules, in particular viral antigens, with antigens from various coronavirus variants being a specific example.

### FIELD OF THE INVENTION

The present invention relates to production of multiple immune-active molecules in genetically modified ascomycetous filamentous fungi, in particular of the species Thermothelomyces heterothallica (formerly Myceliophthora thermophila). In particular, the genetically modified ascomycetous filamentous fungi are used for robust and fast production of multiple antigens, and in particular glycosylated antigens, with antigens from various coronavirus variants being a specific example.

### BACKGROUND OF THE INVENTION

Wild type Thermothelomyces heterothallica (Th. heterothallica) C1 (recently renamed from Myceliophthora thermophila, which in term was renamed from Chrysosporium lucknowense) is a thermotolerant ascomycetous filamentous fungus producing high levels of cellulases, which made it attractive for production of these and other proteins on a commercial scale.

For example, U.S. Pat. Nos. 8,268,585 and 8,871,493 to the Applicant of the present invention disclose a transformation system in the field of filamentous fungal hosts for expressing and secreting heterologous proteins or polypeptides. Also disclosed is a process for producing large amounts of polypeptides or proteins in an economical manner. The system comprises a transformed or transfected fungal strain of the genus Chrysosporium, more particularly of Chrysosporium lucknowense and mutants or derivatives thereof. Also disclosed are transformants containing Chrysosporium coding sequences, as well expressing-regulating sequences of Chrysosporium genes.

Wild type C1 was deposited in accordance with the Budapest Treaty with the number VKM F-3500 D, deposit date Aug. 29, 1996. High Cellulase (HC) and Low Cellulase (LC) strains have also been deposited, as described, for example, in U.S. Pat. No. 8,268,585.

Recently, the Applicant of the present application has shown that filamentous fungi, particularly Th. heterothallica is highly suitable for the production of secondary metabolites. International (PCT) Application Publication No. WO 2020/161682 discloses that Th. heterothallica is capable of producing cannabinoids and precursors thereof, particularly of producing cannabigerolic acid (CBGA) and/or cannabigerovarinic acid (CBGVA) and products thereof, including tetrahydrocannabinolic acid (THCA), cannabidiolic acid (CBDA) and cannabidivarinic acid (CBDVA), and use thereof for producing said precursors and cannabinoids.

### Coronavirus

Coronaviruses (CoVs) are the largest group of viruses belonging to the Nidovirales order, which includes Coronaviridae, Arteriviridae, and Roniviridae families. The Coronavirinae comprise one of two subfamilies in the Coronaviridae family, with the other being the Torovirinae. Coronaviruses are associated with illness from the common cold to more severe conditions such as Severe Acute Respiratory Syndrome (SARS-CoV) and Middle East Respiratory Syndrome (MERS-CoV). Severe acute respiratory syndrome coronavirus 2 (SARS-CoV-2) is the positive-sense, single-stranded RNA coronavirus that causes the coronavirus disease 2019 (COVID-19). Coronaviruses are zoonotic, meaning they are transmitted between animals and people. Common signs of coronavirus infection include respiratory symptoms, fever, coughing, shortness of breath and breathing difficulties. High concentrations of cytokines were recorded in plasma of critically ill patients infected with COVID-19. In more severe cases, infection can cause pneumonia, respiratory inflammation, severe acute respiratory syndrome, kidney failure and death. Recombinant production of viral proteins may be used as potential vaccine. Coronavirus spike proteins are considered as a major target for vaccine development.

SARS-CoV-2 has many variants which raise concerns due to their potential for increased transmissibility, increased virulence, or reduced effectiveness of vaccines against them.

There remains a need for expression systems for mass production of immune-active molecules, including antigens (coronavirus antigens being an example) and antibodies, that can be used in the pharmaceutical industry in an efficient and cost-effective way. In particular, there is a need for improved and robust expression systems that can produce a variety of coronavirus antigens individually or two, three, four or more antigens simultaneously for vaccination.

### SUMMARY OF THE INVENTION

The present invention provides genetically modified ascomycetous filamentous fungi capable of producing a plurality of immune-active molecules. The present invention further provides in some embodiments genetically modified ascomycetous filamentous fungi capable of producing a plurality of viral antigens. The present invention provides in some embodiments genetically modified ascomycetous filamentous fungi capable of producing multiple viral antigens, such as receptor binding domains (RBD) of different SARS-COV-2 variants. The present invention further provides in some embodiments genetically modified ascomycetous filamentous fungi capable of producing high amounts of RBD variants fused to a C-tag. The present invention further provides a cultivation mix comprising two or more genetically modified ascomycetous filamentous fungi, each expressing a different antigen. The present invention further provides in some embodiments genetically modified ascomycetous filamentous fungi capable of producing antibodies or fragments thereof, each encoded by a polynucleotide located in a different site within the fungal genome.

In particular, the present invention provides Thermothelomyces heterothallica strain C1 as an exemplary ascomycetous filamentous fungus genetically modified to produce a plurality of SARS-COV-2 receptor binding domain (RBD) of different coronavirus variants. This enables fast and efficient production of a “cocktail” of antigens that can serve for vaccination.

Surprisingly, the present invention shows that Th. heterothallica, exemplifying ascomycetous filamentous fungi, can be genetically modified to produce simultaneously robust amount of two, and even three or more, different antigens of coronavirus.

Advantageously, the genetically modified ascomycetous filamentous fungus of the invention was designed, in some embodiments, to produce secreted viral antigens. The secretion of the expressed proteins and the prevention of fragmented proteins in the medium simplify the purification procedure and increase the protein yield.

It is further disclosed that the antigens described in the present invention are inserted to well determined loci within the fungal genome (landing sites) that can be reused for replacing antigens with other antigens as needed.

According to one aspect, the present invention provides a genetically modified filamentous fungus comprising at least one cell capable of producing at least two different immune-active molecules, said immune-active molecules are produced from at least two different transcription units, wherein the ratio between the amounts of the at least two immune-active molecules produced is from about 1:1 to about 1:10.

According to some embodiments, the immune-active molecules are antigens. According to certain embodiments, the antigen is a viral antigen.

According to some embodiments, the immune-active molecules are antibodies or a fragment thereof. According to certain embodiments, the antibody fragment comprises the biding site of the antibody. According to some embodiments, the immune-active molecules are multi-specific antibodies. According to certain embodiments, the antibody is a bispecific antibody or trispecific antibody. According to certain embodiments, the immune-active molecules are in the form of nanobodies or Fc-fusion molecules.

According to other embodiments, the immune-active molecules are therapeutic proteins that affect the immune system. According to exemplary embodiments, the therapeutic protein is insulin.

According to certain exemplary embodiments, the antibody is Nivolumab.

According to additional embodiments, the immune-active molecules comprise a combination of antigens and antibodies.

According to some embodiments, the polynucleotides encoding the immune-active molecules are randomly inserted to the genome of the fungus. According to other embodiments, the polynucleotides encoding the immune-active molecules are inserted to specific loci within the fungal genome. According to certain embodiments, the polynucleotides encoding the immune-active molecules are inserted to specific loci within the genome using the CRISPR system.

According to additional embodiments, at least one of the transcription units comprises more than one copy of the immune-active molecule.

According to certain exemplary embodiments, at least one of the polynucleotides encoding the immune-active molecules is localized to a locus selected from the group consisting of cellobiohydrolase (cbh1), β-glucosidase (bgl8), cellobiose dehydrogenase (cbd), chitinase (chi1), a glycoside hydrolase family 6 gene (GH6), a glycoside hydrolase family 61 gene (GH61), a carbohydrate-binding WSC, and any combination thereof, in the fungal genome.

According to some embodiments, at least one of the polynucleotides encoding the immune-active molecules is introduced to specific locus within the fungal genome using a CRISPR RNA (crCRISPR) comprising a sequence selected from the group consisting of SEQ ID Nos: 67-80.

According to certain aspects, the present invention provides nanoparticles comprising the immune-active molecules produced by the genetically modified filamentous fungus as described herein.

According to some embodiments, the immune-active molecules are antigens. According to certain exemplary embodiments, the antigens are viral antigens.

According to some embodiments, the nanoparticles are ferritin nanoparticles. According to certain embodiments, the present invention provides a genetically modified filamentous fungus comprising at least one cell capable of producing at least two different viral antigens, said antigens are produced from at least two different transcription units, wherein the ratio between the amounts of the at least two antigens produced is from about 1:1 to about 1:10.

According to some embodiments, the viral antigens are selected from the group consisting of coronavirus, influenza virus, hepatitis B, hepatitis C, papillomavirus, HIV, HTLV-1, and EBV antigens.

According to some embodiments, the viral antigen is hemagglutinin or neuraminidase.

According to some embodiments, at least one of the viral antigens is of a coronavirus. According to certain embodiments, at least one of the antigens is a spike protein or a fragment thereof. According to certain embodiments, at least one of the antigens comprise the coronavirus receptor binding domain (RBD) or a fragment thereof.

According to certain embodiments, the viral antigens are coronavirus antigens. According to certain currently exemplary embodiments, the coronavirus is SARS-COV-2 (COVID-19).

According to some embodiments, the antigens are spike proteins, or a fragment thereof. According to some embodiments, the antigens comprise the full length of the spike proteins. According to some embodiments, the antigens comprise the S1 subunit of the spike protein.

According to certain embodiments, the antigens comprise the RBD, or a fragment thereof. According to certain embodiments, the fungal cell is capable of producing two different coronavirus RBDs, or fragments thereof.

According to some embodiments, the fungal cell is capable of producing at least two RBD antigens or fragments thereof, of two different SARS-COV-2 variants.

According to some embodiments, at least one of the antigens is of SARS-COV-2 variant B.1.1.7-UK. According to certain embodiments, at least one of the antigens is the RBD of SARS-COV-2 variant B.1.1.7-UK set forth in SEQ ID NO: 2 or a fragment thereof.

According to some embodiments, at least one of the antigens is of SARS-COV-2 variant B.1.351-SA. According to certain embodiments, at least one of the antigens is the RBD of SARS-COV-2 variant B.1.351-SA set forth in SEQ ID NO: 4 or a fragment thereof.

According to some embodiments, at least one of the antigens is of SARS-COV-2 variant 1.1.28.1(P.1)-BR. According to certain embodiments, at least one of the antigens is the RBD of SARS-COV-2 variant 1.1.28.1(P.1)-BR set forth in SEQ ID NO: 6 or a fragment thereof.

According to some embodiments, at least one of the antigens is of SARS-COV-2 variant DELTA variant (B.1.617.2).

According to some embodiments, at least one of the antigens is RBD, or a fragment thereof, of Wuhan variant, B.1.1.7-UK variant, B.1.351-SA variant, 1.1.28.1(P.1)-BR variant, Omicron BA.4 or Omicron BA.5.

According to some embodiments, the at least two viral antigens are RBD sequences or a fragment thereof, of two different SARS-COV-2 variants selected from the group consisting of: (1) Wuhan variant and B.1.1.7-UK; (2) Wuhan variant and B.1.351-SA; (3) Wuhan variant and 1.1.28.1(P.1)-BR; (4) B.1.1.7-UK and B.1.351-SA; (5) B.1.1.7-UK and 1.1.28.1(P.1)-BR, and (6) B.1.351-SA and 1.1.28.1(P.1)-BR. Each possibility represents a separate embodiment of the invention.

According to some embodiments, the at least two viral antigens are RBD sequences or a fragment thereof, of two different SARS-COV-2 variants selected from the group consisting of: (1) DELTA variant (B.1.617.2) and B.1.1.7-UK; (2) DELTA variant (B.1.617.2) and B.1.351-SA; (3) DELTA variant (B.1.617.2) and 1.1.28.1(P.1)-BR; (4) DELTA variant (B.1.617.2) and Wuhan variant, and (5) Omicron BA.4 and Omicron BA.5. Each possibility represents a separate embodiment of the invention.

According to some embodiments, the fungal cell is capable of producing three viral antigens. According to certain embodiments, the fungal cell is capable of producing three viral antigens, each of a different viral variant. According to additional embodiments, the fungal cell is capable of producing four viral antigens, each of a different viral variant.

According to certain embodiments, the fungal cell is capable of producing two, three or more copies of the same variant.

According to some embodiments, the viral antigens are inserted into two, three, four, or more different loci (‘landing sites’) within the fungal genome. According to certain exemplary embodiments, one of the loci is chitinase locus, wherein the polynucleotide encoding the antigen(s) replaces the endogenous chitinase or part thereof.

According to some embodiments, the antigens are from different viruses. According to certain embodiments, the antigens are from coronavirus and influenza. According to certain exemplary embodiments, the antigens are RBD and hemagglutinin or neuraminidase, their fragments or any combination thereof.

According to some embodiments, the fungal cell is capable of producing three viral antigens from two different transcription units. According to some embodiments, the fungal cell is capable of producing three viral antigens from three different transcription units. According to additional embodiments, the fungal cell is capable of producing four viral antigens from two, three or four different transcription units. Each possibility represents a separate embodiment of the invention.

According to some embodiments, the fungal cell is capable of producing three different coronavirus antigens. According to certain embodiments, the fungal cell is capable of producing three different coronavirus antigens. According to certain embodiments, the fungal cell is capable of producing RBD antigens, or a fragment thereof, of three different SARS-COV-2 variants. According to certain exemplary embodiments, the fungal cell is capable of producing RBD antigens, or a fragment thereof of the three SARS-COV-2 variants B.1.1.7-UK, B.1.1.7-UK and 1.1.28.1(P.1)-BR.

According to some embodiments, the fungal cell is capable of producing RBD antigens, or a fragment thereof, of four different SARS-COV-2 variants.

According to additional embodiments, at least one of the antigens comprises the receptor binding motif (RBM) of SARS-CoV-2 spike protein.

According to some embodiments, the RBD fragment length is at least 150 amino acids. According to some embodiments, the RBD fragment length is at least 100 amino acids. According to certain embodiments, the RBD fragment length is between about 100 and 180 amino acids. According to additional embodiments, the RBD comprises at least the receptor-binding motif (RBM).

According to some embodiments, the ratio between the amounts of the at least two antigens is between 1:1 and 1:10, between 1:1 and 1:8, between 1:1 and 1:6, between 1:1 and 1:4 or between 1:1 and 1:2. Each possibility represents a separate embodiment of the invention.

It is to be understood that if three or more different antigens are produced the indicated ratios relate to at least two of them. According to some embodiments, the fungal produce 3 or more viral antigens, and the ratio between the amounts of the antigen having the highest amount and the antigen having the lowest amount does not exceed 10, 9, 8, 7, 6, 5, 4, or 3. Each possibility represents a separate embodiment of the invention.

According to some embodiments, the fungal cell is capable of producing RBD antigens, or a fragment thereof, of three different SARS-COV-2 variants, and the ratio between any two of them does not exceed 10. According to some embodiments, the fungal cell is capable of producing RBD antigens, or a fragment thereof, of three different SARS-COV-2 variants, and the ratio between any two of them does not exceed 5.

According to some embodiments, the antigens are glycosylated.

According to some embodiments, the modified filamentous fungus comprises at least one cell having reduced expression and/or activity of a protease.

According to some embodiments, the modified filamentous fungus comprises at least one cell having reduced expression and/or activity of at least 2, 3, 4, 5, 6, 7, 8, 9, 10, 11, 12, 13, or 14 proteases. Each possibility represents a separate embodiment of the invention According to some embodiments, the modified filamentous fungus comprises at least one cell having reduced expression and/or activity of KEX2 and/or ALP7. According to some embodiments, the modified filamentous fungus comprises at least one cell having reduced expression and/or activity of KEX2 and ALP7, and at least 1, 2, 3, 4, 5, 6, 7, 8, 9, 10, 11, or 12 additional proteases.

According to some embodiments, the at least one additional protease is selected from the group consisting of ALP1, PEP4, ALP2, PRT1, SRP1, ALP3, PEP1, MTP2, PEP5, MTP4, PEP6, and ALP4. Each possibility represents a separate embodiment of the invention.

According to some embodiments, the at least one additional protease is selected from the group consisting of ALP1, PEP4, ALP2, PRT1, SRP1, ALP3, PEP1, MTP2, PEP5, MTP4, PEP6, ALP4, ALP5, ALP6, SRP3, SRP5, SRP8 and SRP10.

According to some embodiments, the modified filamentous fungus comprises at least one cell having reduced expression and/or activity of ALP1, PEP4, ALP2, PRT1, SRP1, ALP3, PEP1, MTP2, PEP5, MTP4, PEP6, ALP4, KEX2 and ALP7.

According to some embodiments, the modified filamentous fungus comprises at least one cell having reduced expression and/or activity of ALP1, PEP4, ALP2, PRT1, SRP1, ALP3, PEP1, MTP2, PEP5, MTP4, PEP6, ALP4, ALP7 and SRP10.

According to some embodiments, at least one of the antigens is a secreted protein. According to some embodiments, the at least two antigens are secreted proteins. According to some embodiments, at least one of the antigens comprises a signal peptide for secretion. According to additional embodiments, the at least two antigens have a leader or a signal peptide. According to other embodiments, at least one of the antigens is an intracellular protein.

According to some embodiments, at least one of the antigens is fused to a tag. According to some embodiments, the tag is a C-terminal or N-terminal tag. According to some embodiments, the tag is selected from the group consisting of chitin binding protein (CBP), maltose binding protein (MBP), Strep-tag, glutathione-S-transferase (GST), FLAG-tag, Spytag, C-tag, ALFA-tag, V5-tag, Myc-tag, HA-tag, Spot-tag, T7-tag, NE-tag, and poly(His) tag. According to some embodiments, the tag is Spytag. According to some embodiments, the tag is C-tag.

According to some embodiments, each of the at least two antigens is fused to a tag. According to certain embodiments, each of the at least two antigens is fused to a different tag.

According to some embodiments, the modified filamentous fungus further comprises a polynucleotide encoding a viral antigen of a different virus.

According to certain exemplary embodiments, the different virus is of a coronavirus, influenza virus, hepatitis B, hepatitis C, papillomavirus, HIV, HTLV-1, or EBV.

According to some embodiments, at least one of the antigens is fused to an Fc fragment. According to certain embodiments, the Fc is fused to the N terminus of the antigen. According to other embodiments, the Fc is fused to the C terminus of the antigen.

According to some embodiments, at least one of the antigens is fused to an MHCII targeting sequence. According to certain embodiments, the antigen and the MHCII targeting sequence are connected via a linker.

According to certain embodiments, the genetically modified ascomycetous filamentous fungus is capable of producing at least 1 gr of each immune-active molecule per a batch of Liter fermentation.

According to some embodiments, the genetically modified ascomycetous filamentous fungus is capable of producing each immune-active molecule at an amount of at least 0.04 gr per gr dry weight.

According to certain embodiments, the genetically modified ascomycetous filamentous fungus is capable of producing at least 0.1 gr of each viral antigen per a batch of Liter fermentation. According to certain embodiments, the genetically modified ascomycetous filamentous fungus is capable of producing at least 0.2 gr of each viral antigen per a batch of Liter fermentation. According to additional embodiments, the genetically modified ascomycetous filamentous fungus is capable of producing at least 0.5 gr of each viral antigen per a batch of Liter fermentation, 1 gr of each viral antigen per a batch of Liter fermentation, 1.5 gr of each viral antigen per a batch of Liter fermentation or 2 gr of each viral antigen per a batch of Liter fermentation.

According to additional embodiments, the genetically modified ascomycetous filamentous fungus is capable of producing at least 3, 4, 5, 6, 7, 8, 9, 10, 12, 15, or 20 gr of each viral antigen per a batch of Liter fermentation. Each possibility represents a separate embodiment of the invention.

According to some embodiments, the genetically modified ascomycetous filamentous fungus is capable of producing the immune-active molecule at an amount of at least 0.04, 0.08, 0.1, 0.2, 0.3, or 0.4 gr per gr dry weight. Each possibility represents a separate embodiment of the invention.

According to some embodiments, the at least two antigens are encoded by at least two transcription units that are integrated into the genome of the ascomycetous filamentous fungus.

According to some embodiments, the at least two transcription units are comprised within at least one exogenous polynucleotide, said polynucleotide is a DNA construct or an expression vector further comprising at least one regulatory element operable in said ascomycetous filamentous fungus. According to certain embodiments, the regulatory element is selected from the group consisting of a regulatory element endogenous to said fungus and a regulatory element heterologous to said fungus.

According to some embodiments, the ascomycetous filamentous fungus is of a genus within the group Pezizomycotina.

According to some embodiments, the ascomycetous filamentous fungus is of a genus selected from the group consisting of Thermothelomyces, Myceliophthora, Trichoderma, Aspergillus, Penicillium, Rasamsonia, Chrysosporium, Corynascus, Fusarium, Neurospora, and Talaromyces.

According to some embodiments, the ascomycetous filamentous fungus is of a species selected from the group consisting of Thermothelomyces heterothallica (also denoted Myceliophthora thermophila), Myceliophthora lutea, Aspergillus nidulans, Aspergillus funiculosus Aspergillus niger, Aspergillus oryzae, Trichoderma reesei, Trichoderma harzianum, Trichoderma longibrachiatum, Trichoderma viride, Rasamsonia emersonii. Penicillium chrysogenum, Penicillium verrucosum, Sporotrichum thermophile, Corynascus fumimontanus, Corynascus thermophilus, Chrysosporium lucknowense, Fusarium graminearum, Fusarium venenatum, Neurospora crassa, and Talaromyces piniphilus.

According to some embodiments, the ascomycetous filamentous fungus is a Thermothelomyces heterothallica strain comprising rDNA sequence having at least 95%, or at least 96%, or at least 97%, or at least 98%, or at least 99% or 100% identity to the nucleic acid sequence set forth in SEQ ID NO: 1.

According to some embodiments, the ascomycetous filamentous fungus is Thermothelomyces heterothallica C1.

According to an aspect, the present invention provides a method for producing a fungus capable of producing at least two different immune active molecules, the method comprising integrating to the fungus genome at least one exogenous polynucleotide encoding for two different transcription units, each is capable of translating a different immune active molecule.

According to an aspect, the present invention provides a method for producing a fungus capable of producing at least two different viral antigens, the method comprising integrating to the fungus genome at least one exogenous polynucleotide encoding for two different transcription units, each is capable of translating a different viral antigen.

According to an additional aspect, the present invention provides a method for producing a fungus capable of producing at least two viral antigens, the method comprising transforming at least one cell of the fungus with at least two exogenous polynucleotides, wherein each polynucleotide comprises a sequence encoding for a different viral antigen.

According to some embodiments, the at least two exogenous polynucleotides encode three different immune-active molecules. According to some embodiments, the at least two exogenous polynucleotides encode four different immune-active molecules.

According to some embodiments, the at least two exogenous polynucleotides encode three different viral antigens. According to some embodiments, the at least two exogenous polynucleotides encode four different viral antigens.

The viral antigens and the ascomycetous filamentous fungus are as describe hereinabove. According to certain embodiments, the viral antigen is a coronavirus antigen.

According to a further aspect, the present invention provides a method of producing a composition comprising at least two different viral antigens, the method comprising culturing the genetically modified fungus as described herein in a suitable medium; and recovering the at least two viral antigens products.

According to some embodiments, the recovering step comprises recovering the at least two antigens from the growth medium, from the fungal mass or both.

According to some embodiments, at least two of the antigens are secreted and recovered from the growth medium. According to certain embodiment, at least 50%, 60%, 70%, 80%, 90% or 95% of the antigens are secreted.

According to some embodiments, the medium comprises a carbon source selected from the group consisting of glucose, sucrose, xylose, arabinose, galactose, fructose, lactose, cellobiose, glycerol and any combination thereof.

According to another aspect, the present invention provides a plurality of different viral antigens produced by any of the methods described herein.

According to some embodiments, the antigens are coronavirus antigens. According to certain embodiments, the antigens comprise a coronavirus RBD sequence or a fragment thereof.

The present invention further provides a composition comprising two or more different antigens produced by the fungus and/or any of the methods described herein.

According to certain embodiments, the composition comprises at least two different coronavirus antigens, said antigens comprises sequences of different coronavirus variants.

According to an additional aspect, the present invention provides a genetically modified filamentous fungus comprising at least one cell modified to produce multiple copies of viral antigens.

According to certain embodiments, the antigens are coronavirus antigens. According to certain exemplary embodiments, the antigens are RBD antigens.

According to some embodiments, the antigens are influenza antigens. According to certain embodiments, the antigens are of influenza A or influenza B. According to certain embodiments, the antigens are hemagglutinin or neuraminidase.

According to some embodiments, the antigens are of different viruses. In certain exemplary embodiments, the antigens are of coronavirus and influenza.

According to another aspect, the present invention provides a cultivation comprising a mix of at least two genetically modified filamentous fungi as described herein, each is capable of producing a different immune-active molecule.

According to another aspect, the present invention provides a cultivation comprising a mix of at least two genetically modified filamentous fungi as described herein, each is capable of producing a different viral antigen.

According to another aspect, the present invention provides a cultivation comprising a mix of at least two genetically modified filamentous fungi, each fungus comprising at least one cell capable of producing a viral antigen, and wherein the at least two fungi are capable of producing a different viral antigen.

According to some embodiments, the ratio between the amounts of the produced immune-active molecules is between 1:1 and 1:10. According to some embodiments, the ratio between the amounts of the produced immune-active molecules is between 1:1 and 1:5. According to some embodiments, the ratio between the amounts of the produced immune-active molecules is between 1:1 and 1:2.

According to some embodiments, the ratio between the amounts of the produced antigens is between 1:1 and 1:10. According to some embodiments, the ratio between the amounts of the produced antigens is between 1:1 and 1:5. According to some embodiments, the ratio between the amounts of the produced antigens is between 1:1 and 1:2.

According to some embodiments, the cultivation comprising a mix of at least three genetically modified filamentous fungi capable of producing at least three different antigens.

The fungi and the antigen are as described hereinabove. According to specific embodiments, the antigen is a coronavirus antigen. According to additional embodiments, the antigen is RBD.

According to some embodiments, each of the genetically modified filamentous fungus is capable of producing a different RBD variant.

According to some embodiments, the genetically modified filamentous fungus has reduced expression and/or protease activity of at one or more proteases as described herein. According to certain embodiments, the genetically modified filamentous fungus has reduced expression and/or protease activity of ×13 or ×14 proteases.

According to another aspect, the present invention provides a genetically modified filamentous fungus comprising at least one cell modified to produce at least two antibodies or fragments thereof.

According to some embodiments, each of the antibodies or fragments thereof is encoded by a polynucleotide located in a different site within the fungal genome.

According to some embodiments, the fungus is capable of producing a light chain and a heavy chain of an antibody, each chain is produced by a different transcription unit. According to certain embodiments, the antibodies or fragments thereof are encoded by polynucleotides, each is integrated at a separate site within the genome of the fungus.

According to another aspect, the present invention provides a genetically modified filamentous fungus comprising at least one cell modified to produce an RBD antigen fused to a c-Tag, said cell has reduced expression and/or protease activity of ×13 or ×14 proteases.

According to another aspect, the present invention provides a genetically modified filamentous fungus comprising at least one cell modified to produce an RBD_of B.1.1.7-UK, RBD_B.1.351-SA, RBD_1.1.28.1(P.1)-BR, RBD_1.617.2-DELTA, Omicron BA.4, or Omicron BA.5.

According to additional aspect, the present invention provides a genetically modified filamentous fungus comprising at least one cell capable of producing at least two immune-active molecules, said immune-active molecules are produced from at least two different transcription units.

According to some embodiments, genetically modified filamentous fungus is capable of producing at least 0.2 gr of said immune-active molecules per 1 gr of fungal dry weight. According to certain embodiments, genetically modified filamentous fungus is capable of producing at least 0.25, 0.3, 0.35, 0.4 or 0.45 gr of said immune-active molecules per 1 gr of fungal dry weight. Each possibility represents a separate embodiment of the invention.

According to some embodiments, the at least two immune-active molecules are identical. According to some embodiments, the at least two immune-active molecules are different.

According to some embodiments, the immune-active molecules are antibodies. According to some embodiments, the immune-active molecules are antigens. According to some embodiments, the immune-active molecules are viral antigens. According to some embodiments, the immune-active molecules comprise antibodies and antigens. According to some embodiments, the viral antigen is a component of an epidemic virus. According to certain exemplary embodiments, the viral component is of a coronavirus, an influenza virus, hepatitis B, hepatitis C, papillomavirus, HIV, HTLV-1, or EBV. Each possibility represents a separate embodiment of the invention

According to another aspect, the present invention provides a genetically modified filamentous fungus comprising at least one cell capable of producing at least two heterologous proteins, said heterologous proteins are produced from at least two different transcription units, wherein the cell comprises at least one polynucleotide encoding the heterologous protein, said polynucleotide is localized to a locus selected from the group consisting of cellobiohydrolase (cbh1), β-glucosidase (bgl8), cellobiose dehydrogenase (cbd), chitinase (chi1), a glycoside hydrolase family 6 gene (GH6), a glycoside hydrolase family 61 gene (GH61), a carbohydrate-binding WSC, and any combination thereof, in the fungal genome.

According to some embodiments, the ratio between the amounts of the at least two proteins produced is from about 1:1 to about 1:10. According to some embodiments, the ratio between the amounts of the at least two proteins produced is from about 1:1 to about 1:5, or from about 1:1 to about 1:2.

According to additional embodiments, at least one of the transcription units comprises more than one copy of the proteins.

According to some embodiments, at least one of the polynucleotides encoding the proteins is introduced to specific locus within the fungal genome using a CRISPR RNA (crRNA) comprising a sequence selected from the group consisting of SEQ ID Nos: 67-80.

According to some embodiments, the genetically modified ascomycetous filamentous fungus is capable of producing the heterologous protein at an amount of at least 0.02 gr per 1 gram of fungal dry weight. According to additional embodiments, the genetically modified ascomycetous filamentous fungus is capable of producing the heterologous protein at an amount of at least 0.025, 0.03, 0.035, or 0.04 gr per 1 gram of fungal dry weight. According to certain exemplary embodiments, the fungus is capable of producing the heterologous protein at an amount of at least 0.048 gram per 1 gram of fungal dry weight.

It is to be understood explicitly that the scope of the present invention encompasses homologs, analogs, variants and derivatives, including shorter and longer polypeptides, proteins and polynucleotides, as well as polypeptide, protein and polynucleotide analogs with one or more amino acid or nucleic acid substitution as are known in the art, with the stipulation that these variants and modifications must preserve the activity of the antigens described herein.

It is to be understood that any combination of each of the aspects and the embodiments disclosed herein is explicitly encompassed within the disclosure of the present invention.

Other objects, features and advantages of the present invention will become clear from the following description and drawings.

### BRIEF DESCRIPTION OF THE FIGURES

FIG. 1. Western blotting result from 24-well cultivations of several positive transformants for the expression of Wuhan RBD-C-tag. Signal of the expected size is detected with the anti-C-tag antibody.

FIG. 2. C-tag affinity purification of Wuhan RBD-C-tag from a bioreactor cultivation of C1 strain M4169. Stained SDS-PAGE and Western analysis of samples from different purification steps are shown. Start=Start sample after clarification; Flow start=flow through in the beginning of sample loading; Flow end=flow through at the end of sample loading; Wash=washing step; Fr4-Fr9=elution fractions; F6-Fr9=pooled fractions before dialysis; Final dialyzed=dialyzed sample, four different sample volumes.

FIG. 3. Repligen v1 affinity purification of Wuhan-RBD-C-tag from a bioreactor cultivation of C1 strain M4169. Stained SDS-PAGE and Western analysis of samples from different purification steps are shown. S=Start sample after clarification; Flow1=flow through in the beginning of sample loading; Flow2=flow through at the end of sample loading; Wash=washing step; Fr4-Fr10=elution fractions; Dialyzed Fr5-7=Dialyzed sample pool of fractions 5-7, three different sample volumes; Dialyzed Fr7-9=Dialyzed sample pool of fractions 7-9, three different sample volumes.

FIG. 4. Stained SDS-PAGE result from 24-well cultivations of several positive transformants for the expression of Wuhan-RBD-C-tag from two gene copies. M4169 is shown as a control for expression of Wuhan-RBD-C-tag from a single gene copy.

FIG. 5. Western blotting and stained SDS-PAGE results result from 24-well cultivations of several positive transformants for expression of Wuhan RBD without C-tag. M4169 is show as a control for expression of Wuhan-RBD-C-tag. Signal of the expected size is detected both in stained SDS-PAGE and in Western blotting (anti-RBD antibody). As expected, RBD without the C-tag is slightly smaller than RBD-C-tag.

FIG. 6. Western blotting result from 24-well plate culture of C1 transformants producing alpha-UK, beta-SA and gamma-BR RBD-C-tag variants. Shown is the overlay signal of both anti-RBD and anti-C-tag detection agents. The sample denoted Wuhan is from the M4169 C1 strain producing Wuhan RBD.

FIG. 7. Western blotting result from 24-well cultivations with several positive transformants for all single-copy versions. Signals of the expected size are detected both with anti-RBD and anti-C-tag antibodies from Delta-C-tag and beta-SA-C-tag transformants shown is the overlay signal of both anti-RBD signal and anti-C-tag) and with anti-RBD antibody from Delta and beta-SA transformants.

FIG. 8. RBD affinity purification of RBD-Delta from a bioreactor cultivation of C1 strain M5517. Stained SDS-PAGE and Western analysis with anti-RBD antibody of samples from different purification steps are shown. S=Start sample after clarification; FT=flowthrough of sample loading; W1-W3=washing steps; A7-B3=elution fractions; A8-B2=pooled fractions before dialysis; Dial./R=Dialyzed sample, reducing conditions; Dial./NR=Dialysed sample in non-reducing conditions.

FIG. 9. Western blotting result from 24-well cultivations of several positive transformants for the co-expression of alpha-UK, beta-SA and gamma-BR RBD-C-tag. Shown is the overlay signal of both anti-RBD and anti-C-tag detection agents. The sample denoted M5273 is the C1 strain expressing only gamma-BR.

FIG. 10. Affinity purification of mix of three RBD-C-tag variants with Repligen v1, Repligen v2 and C-tag resins from a bioreactor cultivation of C1 strain M5407. Stained SDS-PAGE and Western analysis of samples from different purification steps are shown. Start=Start sample after clarification; Flow=flow through of sample loading; Fr4 and Fr5=elution fractions.

FIG. 11. Repligen v2 affinity purification of mix of three RBD-C-tag variants from a bioreactor cultivation of C1 strain M5407. Stained SDS-PAGE and Western analysis of samples from different purification steps are shown. Start=Start sample after clarification; Flow=flow through of sample loading; Wash1-Wash3=washing steps; B4-C3=elution fractions; Pooled B4-C3=pooled fractions before dialysis; Final dialyzed=dialyzed sample.

FIG. 12A-12H. ACE2-binding ELISA results of purified RBD-C-tag proteins shown as plotting the mean absorbance 450 nm against the RBD-C-tag concentration. A. Wuhan, B. alpha-UK, C. beta-SA, D. gamma-BR, E. delta-C-tag, F. mix of alpha-UK/beta-SA/gamma-BR, G. omicron B.1.1529, and H. omicron BA.5. Result of reference RBD-C-tag is shown in each graph as solid symbols and test RBD-C-tag proteins are shown as open symbols.

FIG. 13. A schematic description of integration of three different coronavirus variants to the C1 genome according to certain embodiments of the invention.

FIG. 14A-14E. Production and C-tag affinity purification of omicron variants B.1.1.529 and BA.5. A. Western blotting result from 24-well cultivations of three positive transformants expressing omicron B.1.1.529. B. Western blotting result from 24-well cultivations of a transformant expressing omicron BA.5 cultivated at 28° C. and at 35° C. C. Bioreactor cultivation of C1 M5890 strain expressing omicron B.1.1529 at 25° C. and at 38° C. D. C-tag affinity purification of omicron B.1.1.529 from a bioreactor cultivation of C1 strain M5890. Stained SDS-PAGE and Western analysis of samples from different purification steps are shown. Final purified protein (reduced and non-reduced samples) is marked with frames. E. C-tag affinity purification of omicron BA.5 from a bioreactor cultivation of C1 strain M6369. Stained SDS-PAGE and Western analysis of samples from different purification steps are shown. Final purified protein (reduced and non-reduced sample) is marked with frame.

### DETAILED DESCRIPTION OF THE INVENTION

The present invention provides alternative, highly efficient systems for producing high amounts of immune-active molecules, such as antigens and antibodies. In particular, the present invention provides systems for producing high amounts of viral antigens, such as coronavirus antigens from different variants. The present invention provides in some embodiments at least two different antigens from different variants of coronavirus, which allows the production of an efficient “cocktail vaccination” to coronavirus produced by a single line of engineered fungus. According to some embodiments, the different antigens are produced using a genetically modified filamentous fungus that is capable of producing said antigens. According to other embodiments, the different antigens are produced using multiple genetically modified filamentous fungi, each is capable of producing a different antigen, and said fungi are grown in a single cultivation.

Advantageously, the present invention provides a platform for rapid and robust production of antigens. For example, the influenza virus is particularly dangerous to public health because of its high rate of mutating. The antigens on the surfaces of the influenza virus are glycoproteins called hemagglutinin and neuraminidase. The hemagglutinin antigen is capable of undergoing rapid changes called antigenic drift that enable the virus to escape detection by antibodies. Small amino acid substitutions in the glycoprotein can change the epitope enough that the antibody doesn't recognize the antigen anymore. Thus, a new flu vaccine is produced every year. The system of the invention is based in part on the filamentous fungus Thermothelomyces heterothallica C1 and particular strains thereof, which have been previously developed as a natural biological factory for protein as well as secondary metabolite production. These strains show high growth rate while keeping low culture viscosity, and are thus highly suitable for continuous growth in fermentation cultures at volumes as high as 100,000-150,000 liters or greater. The present invention in some embodiments provides genetically modified fungi capable of producing at least two different viral antigens. The present invention in some embodiments provides genetically modified fungi capable of producing at least two coronavirus receptor binding domains (RBD) of different coronavirus strains.

### Definitions

Ascomycetous filamentous fungi as defined herein refer to any fungal strain belonging to the group Pezizomycotina. The Pezizomycotina comprises, but is not limited to the following groups:
Sordariales, including genera:
   Thermothelomyces (including species: heterothallica and thermophila),
   Myceliophthora (including the species lutea and unnamed species),
   Corynascus (including the species fumimontanus),
   Neurospora (including the species crassa);
Hypocreales, including genera:
   Fusarium (including the species graminearum and venenatum),
   Trichoderma (including the species reesei, harzianum, longibrachiatum and viride);
Onygenales, including genera:
   Chrysosporium (including the species lucknowense);
Eurotiales, including genera:
   Rasamsonia (including the species emersonii),
   Penicillium (including the species verrucosum),
   Aspergillus (including the species funiculosus, nidulans, niger and oryzae)
   Talaromyces (including the species piniphilus (formerly Penicillium funiculosum));

It is to be understood that the above list is not conclusive, and is meant to provide an incomplete list of industrially relevant filamentous ascomycetous fungal species.

While there may be filamentous ascomycetous species outside Pezizomycotina, that group does not contain Saccharomycotina, which contains most commonly known non-filamentous industrially relevant genera, such as Saccharomyces, Komagataella (including formerly Pichia pastoris), Kluyveromyces or Taphrinomycotina, which contains some other commonly known non-filamentous industrially relevant genera, such as Schizosaccharomyces.

All taxonomical categories above are defined according to the NCBI Taxonomy browser (ncbi.nlm.nih.gov/taxonomy) as of the date of the patent application.

It must be appreciated that fungal taxonomy is in constant move, and the naming and the hierarchical position of taxa may change in the future. However, a skilled person in the art will be able to unambiguously determine if a particular fungal strain belongs to the group as defined above.

According to certain embodiments, the filamentous fungus genus is selected from the group consisting of Myceliophthora, Thermothelomyces, Aspergillus, Penicillium, Trichoderma, Rasamsonia, Chrysosporium, Corynascus, Fusarium, Neurospora, Talaromyces and the like. According to some embodiments, the fungus is selected from the group consisting of Myceliophthora thermophila, Thermothelomyces thermophila (formerly M. thermophila), Thermothelomyces heterothallica (formerly M. thermophila and heterothallica), Myceliophthora lutea, Aspergillus nidulans, Aspergillus funiculosus Aspergillus niger, Aspergillus oryzae, Penicillium chrysogenum, Penicillium verrucosum, Trichoderma reesei, Trichoderma harzianum, Trichoderma longibrachiatum, Trichoderma viride, Chrysosporium lucknowense, Rasamsonia emersonii, Sporotrichum thermophile, Corynascus fumimontanus, Corynascus thermophilus, Fusarium graminearum, Fusarium venenatum, Neurospora crassa, and Talaromyces piniphilus.

In particular, the present invention provides Thermothelomyces heterothallica strain C1 as model for an ascomycetous filamentous fungus, capable of producing high amounts of stable proteins.

The terms “Thermothelomyces” and its species “Thermothelomyces heterothallica and thermophila” are used herein in the broadest scope as is known in the art. Description of the genus and its species can be found, for example, in Marin-Felix Y (2015. Mycologica 107(3): 619-632 doi.org/10.3852/14-228) and van den Brink J et al. (2012, Fungal Diversity 52(1):197-207). As used herein “C1” or “Thermothelomyces heterothallica C1” or Th. heterothallica C1, or C1 all refer to Thermothelomyces heterothallica strain C1.

It is noted that the above authors (Marin-Felix et al., 2015) proposed splitting of the genus Myceliophthora based on differences in optimal growth temperature, morphology of the conidiospore, and details of the sexual reproduction cycle. According to the proposed criteria C1 clearly belongs to the newly established genus Thermothelomyces, which contain former thermotolerant Myceliophthora species rather than to the genus Myceliophthora, which remains to include the non-thermotolerant species. As C1 can form ascospores with some other Thermothelomyces (formerly Myceliophthora) strains with opposite mating type, C1 is best classified as Th. heterothallica strain C1, rather than Th. thermophila C1.

It must also be appreciated that the fungal taxonomy was also in constant change in the past, so the current names listed above may be preceded by a variety of older names beyond Myceliophthora thermophila (van Oorschot, 1977. Persoonia 9(3):403), which are now considered synonyms. For example, Thermothelomyces heterothallica (Marin-Felix et al., 2015. Mycologica, 3:619-63), is synonymized with Corynascus heterotchallicus, Thielavia heterothallica, Chrysosporium lucknowense and thermophile as well as Sporotrichium thermophile (Alpinis 1963. Nova Hedwigia 5:74).

It is further to Be explicitly understood that the present invention encompasses any strain containing a ribosomal DNA (rDNA) sequence that shows 99% homology or more to SEQ ID NO: 1, and all those strains are considered to be conspecific with Thermothelomyces heterothallica.

Particularly, the term Th. heterothallica strain C1 encompasses genetically modified sub-strains derived from the wild type strain, which have been mutated, using random or directed approaches, for example, using UV mutagenesis, or by deleting one or more endogenous genes. For example, the C1 strain may refer to a wild type strain modified to delete one or more genes encoding an endogenous protease. For example, C1 strains which are encompassed by the present invention include strain UV18-25, deposit No. VKM F-3631 D; strain NG7C-19, deposit No. VKM F-3633 D; and strain UV13-6, deposit No. VKM F-3632 D. Further C1 strain that may be used according to the teachings of the present invention include HC strain UV18-100f, deposit No. CBS141147; HC strain UV18-100f, deposit No. CBS141143; LC strain W1L #100I, deposit No. CBS141153; and LC strain W1L #100I, deposit No. CBS141149 and derivatives thereof.

It is to be explicitly understood that the teachings of the present invention encompass mutants, derivatives, progeny, and clones of the Th. heterothallica C1 strains, as long as these derivatives, progeny, and clones, when genetically modified according to the teachings of the present invention are capable of producing at least two antigens according to the teachings of the invention.

According to an aspect of the present invention there is provided a genetically modified filamentous fungus for producing a genetically modified filamentous fungus comprising at least one cell capable of producing at least two different viral antigens, said antigens are produced from at least two different transcription units, wherein the ratio between the amounts of the at least two antigens is between 1:1 and 1:10.

The term “immune active molecule” refers to any compound that may modulate the immune system, such as an antigen or an antibody.

The term “antigen” refers to the binding site or epitope recognized by an antigen-binding polypeptide. The antigen, when introduced into an immunocompetent animal, stimulates the production of a specific antibody or antibodies that can combine with the agent. An antigen may comprise one or more epitopes. As used herein, the term “antigen” also relates to the polypeptide or protein that comprises the antigen region.

The term “transcription unit” as described herein is a region of nucleic acid containing coding sequences and the signals for achieving expression of those coding sequences independently of any other coding sequences.

According to some embodiments, the viral antigens are of coronavirus, influenza A, influenza B, adenovirus, CMV, Coxsackievirus, Dengue Virus, EBV, EV71, Ebola Virus, HAV, HBV, HCMV, HCV, HDV, HEV, HIV, HPV, HSV, HTLV, Japanese Encephalitis, Leukemia Virus, Measles Virus, Orf Virus, Parvovirus, Rabies Virus, Rift Valley Fever Virus, Rubella Virus, Rotavirus, TBEV, Tobacco Etch Virus, Varicella Zoster Virus, Variola, West Nile Virus, Zika Virus, ASFV, Nipah Virus, Norovirus, Yellow Fever Virus, Molluscum Contagiosum virus (pox virus), Respiratory Syncytial Virus, Chikungunya virus, Simian (Macaque) Immunodeficiency Virus, Adeno-Associated Virus (AAV), and any combination thereof. Each possibility represents a separate embodiment of the invention.

According to some embodiments, the viral antigens are of a coronavirus, influenza virus, hepatitis B, hepatitis C, papillomavirus, HIV, HTLV-1, or EBV.

According to certain embodiments, the viral antigens are coronavirus antigens. According to certain currently exemplary embodiments, the coronavirus is SARS-COV-2 (COVID-19).

According to some embodiments, the antigens are spike proteins, or a fragment thereof.

According to certain embodiments, the antigens are coronavirus receptor binding domain (RBD), or a fragment thereof. According to certain embodiments, the fungal cell is capable of producing two different coronavirus RBDs, or fragments thereof.

According to certain embodiments, the fungal cell is capable of producing three viral antigens, each of a different viral variant. According to additional embodiments, the fungal cell is capable of producing four viral antigens, each of a different viral variant.

According to some embodiments, the fungal cell is capable of producing three viral antigens from two different transcription units. According to some embodiments, the fungal cell is capable of producing three viral antigens from three different transcription units. According to additional embodiments, the fungal cell is capable of producing four viral antigens from two, three or four different transcription units. Each possibility represents a separate embodiment of the invention.

According to some embodiments, the fungal cell is capable of producing at least two RBD antigens, or fragments thereof of two different SARS-COV-2 variants.

According to some embodiments, the SARS-COV-2 variant is selected from the group consisting of Wuhan variant, B.1.1.7-UK, B.1.351-SA and 1.1.28.1(P.1)-BR.

RBD_B.1.1.7-UK amino acid sequence is set forth in SEQ ID NO: 2, and DNA sequence in SEQ ID NO: 3.

RBD_B.1.351-SA amino acid sequence is set forth in SEQ ID NO: 4, and DNA sequence in SEQ ID NO: 5.

RBD_1.1.28.1(P.1)-BR amino acid sequence is set forth in SEQ ID NO: 6, and DNA sequence in SEQ ID NO: 7.

According to certain embodiments, the fungal cell is capable of producing three RBD antigens, each of a different SARS-COV-2 variant. According to additional embodiments, the fungal cell is capable of producing four RBD antigens, each of a different SARS-COV-2 variant.

According to some embodiments, the fungal cell is capable of producing three RBD antigens from two different transcription units. According to some embodiments, the fungal cell is capable of producing three RBD antigens from three different transcription units. According to additional embodiments, the fungal cell is capable of producing four RBD antigens from two, three or four different transcription units. Each possibility represents a separate embodiment of the invention.

According to additional embodiments, at least one of the antigens comprises the receptor binding motif (RBM) of SARS-CoV-2 spike protein.

According to some embodiments, the ratio between the amounts of the antigens is between 1:1 and 1:10, between 1:1 and 1:9, between 1:1 and 1:8, between 1:1 and 1:7, between 1:1 and 1:6, between 1:1 and 1:5, between 1:1 and 1:4, between 1:1 and 1:3, or between 1:1 and 1:2. Each possibility represents a separate embodiment of the invention.

According to some embodiments, the modified filamentous fungus comprises at least one cell having reduced expression and/or activity of a protease.

According to some embodiments, the modified filamentous fungus comprises at least one cell having reduced expression and/or activity of at least 2, 3, 4, 5, 6, 7, 8, 9, 10, 11, 12, 13, or 14 proteases. Each possibility represents a separate embodiment of the invention

According to some embodiments, the modified filamentous fungus comprises at least one cell having reduced expression and/or activity of KEX2 and ALP7. According to some embodiments, the modified filamentous fungus comprises at least one cell having reduced expression and/or activity of KEX2 and ALP7, and at least 1, 2, 3, 4, 5, 6, 7, 8, 9, 10, 11, or 12 additional proteases.

The terms “protein” and “polypeptide” are used herein interchangeably and refer to a polymer of amino acids and do not refer to a specific length of the product; thus, peptides, oligopeptides, and polypeptide are included within this definition.

According to some embodiments, the ascomycetous filamentous fungus comprises at least one cell having reduced or abolished expression and/or activity of KEX2 and at least one more protease.

According to some embodiments, the genetically modified filamentous fungus does not express KEX2. According to some embodiments, the genetically modified filamentous fungus does not express ALP7.

According to an aspect of the present invention there is provided a genetically modified ascomycetous filamentous fungus comprising at least one cell capable of producing at least two different viral antigens, said antigens are produced from at least two different transcription units, said genetically modified ascomycetous filamentous fungus does not express or expresses reduced amount of KEX2 and/or ALP7, and at least one additional protease selected form the group consisting of ALP1, PEP4, ALP2, PRT1, SRP1, APL3, PEP1, MTP2, PEP5, MTP4, PEP6, and ALP4.

According to some embodiments, the ratio between the amounts of the at least two antigens is between 1:1 and 1:10.

According to an aspect, the present invention provides a genetically modified ascomycetous filamentous fungus for producing at least two receptor binding domains (RBD) of different SARS-CoV-2 strains, wherein the genetically modified ascomycetous filamentous fungus does not express or expresses reduced amount of KEX2, ALP7, ALP1, PEP4, ALP2, PRT1, SRP1, ALP3, PEP1, MTB2, PEP5, MTP4, PEP6, and ALP4.

The kex2 gene, also known as qds1, srb1, and vmn45, encodes for KEX2 or KEXIN protease. The KEX2 protease is a serine peptidase. The Thermothelomyces heterothallica KEX2 amino acid sequence is set forth in SEQ ID NO: 8.

According to some embodiments, the KEX2 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 8.

The Thermothelomyces heterothallica ALP7 amino acid sequence is set forth in SEQ ID NO: 9.

According to some embodiments, the ALP7 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 9.

According to some embodiments, at least one of the antigens is fused to a tag. According to some embodiments, the tag is a C-terminal or N-terminal tag. According to some embodiments, the tag is selected from the group consisting of chitin binding protein (CBP), maltose binding protein (MBP), Strep-tag, glutathione-S-transferase (GST), FLAG-tag, Spytag, C-tag, ALFA-tag, V5-tag, Myc-tag, HA-tag, Spot-tag, T7-tag, NE-tag, and poly(His) tag. According to some embodiments, the tag is Spytag. According to some embodiments, the tag is C-tag.

As used herein, the term “tag” refers to an amino acid sequence, which is typically in the art fused to or included in another amino acid sequence for a) facilitating purification of the overall amino acid sequence or polypeptide, b) improving expression of the overall amino acid sequence or polypeptide, and/or c) facilitating detection of the overall amino acid sequence or polypeptide.

The term “C-tag” is well known in the art and refers to a 4 amino acid affinity tag: E-P-E-A (glutamic acid-proline-glutamic acid-alanine), which can be fused at the C-terminus of any recombinant protein. The tag offers high affinity and selectivity when used for purification purposes.

The term “Spytag” is well known in the art and refers to a short peptide which binds covalently to SpyCatcher protein. Spytag sequence is Ala-His-Ile-Val-Met-Val-Asp-Ala-Tyr-Lys-Pro-Thr-Lys.

The term “Strep-tag” is used herein as known in the art and refers to a method which allows the purification and detection of proteins by affinity chromatography. The method is based on the Strep-Tactin connection.

The term “Glutathione S-transferases (GSTs)” is used herein as known in the art and is based on the strong binding affinity of the GST protein to glutathione (GSH). A GST-tag is often used to separate and purify proteins that contain the GST-fusion protein. The tag is 220 amino acids in length.

The term “FLAG-tag” is used herein as known in the art and refers to a polypeptide protein tag that can be added to a protein using recombinant DNA technology. It is one of the most specific tags and it is an artificial antigen to which specific, high affinity monoclonal antibodies have been developed and hence can be used for protein purification by affinity chromatography.

The term “ALFA-tag” is used herein as know in the art and refers to an epitope tag that is specifically recognized by a nanobody that can be used for detection and purification.

The V5-tag is a short peptide tag for detection and purification of proteins. The V5 tag can be fused/cloned to a recombinant protein and detected in ELISA, flow cytometry, immunoprecipitation, immunofluorescence, and Western blotting with antibodies and Nanobodies.

The term “Myc-tag” is used herein as known in the art and refers to a short peptide tag derived from the c-myc gene that can be recognized by specific antibodies.

The “HA-tag” is used herein as known in the art and refers to a peptide derived from the Human influenza hemagglutinin (HA) molecule, corresponding to amino acids 98-106. This tag is use to facilitate the detection, isolation, and purification of a protein of interest.

The “Spot-tag” is a 12-amino acid peptide tag recognized by a single-domain antibody nanobody (sdAb). The tag can be used to a variety of applications including: immunoprecipitation, affinity purification, immunofluorescence, and super-resolution microscopy.

The term “T7 tag” is used herein as known in the art and refers to an epitope tag composed of an 11-residue peptide encoded from the leader sequence of the T7 bacteriophage gene 10.

The term “NE-tag” is used herein as known in the art and refers to a synthetic peptide tag (NE tag) designed as an epitope tag for detection, quantification and purification of recombinant proteins. This peptide tag is composed of eighteen hydrophilic amino acids.

The term “poly(His) tag” or “polyhistidine-tag” is as known in the art and refers to an amino acid motif in proteins that typically consists of at least six histidine (His) residues, often at the N- or C-terminus of the protein. It is also known as hexa histidine-tag, 6×His-tag, and His6 tag. The short peptide can be bound by metal ions such as divalent nickel or cobalt.

According to some embodiments, the filamentous fungus is further modified to produce proteins with N-glycans similar to those of human, companion animal and other mammalian proteins. According to some embodiments, the filamentous fungus comprises deletion or disruption of the alg3 gene such that the fungus fails to produce a functional alpha-1,3-mannosyltransferase. According to some embodiments, filamentous fungus comprises deletion or disruption of the alg11 gene such that the fungus fails to produce a functional alpha-1,2-mannosyltransferase. According to some embodiments, the filamentous fungus comprises over-expression of an endogenous flippase or expression of a heterologous flippase.

According to certain embodiments, the filamentous fungus further comprises expression of heterologous GlcNAc transferase 1 (GNT1) and GlcNAc transferase 2 (GNT2). In certain embodiments, the GNT1 comprises a heterologous Golgi localization signal. In some embodiments, the heterologous GNT1 and GNT2 are animal-derived.

According to some embodiments, the antigen is a spike protein. According to some embodiments, the antigen comprises the receptor binding domain (RBD) sequence of SARS-CoV-2 spike protein or a fragment thereof. According to some embodiments, the antigen is the RBD of SARS-CoV-2 spike protein. According to certain embodiments, the antigen comprises the receptor binding motif (RBM) of SARS-CoV-2 spike protein. According to some embodiments, the antigen comprises the glycoprotein-binding domain (GBD) sequence of the SARS-CoV-2 S protein. According to specific embodiments, the RBD or fragment thereof is fused to a Spytag. According to certain embodiments, the RBD or fragment thereof is fused to C tag. According to additional embodiments, the RBD is fused to an Fc of an antibody. According to certain embodiments, the antigen comprises two, three, or four repeats of RBD or a fragment thereof.

The coronavirus antigen sequence can be manipulated according to any known or discovered variant of the coronavirus. For example, the sequence can be manipulated according to a sequence described in Rambaut et al. nCoV-2019 Genomic Epidemiology, Dec. 2020 (virological.org/t/preliminary-genomic-characterisation-of-an-emergent-sars-cov-2-lineage-in-the-uk-defined-by-a-novel-set-of-spike-mutations/563), Tegally, H. et al. 2020 (medrxiv.org/content/10.1101/2020. 12.21.2024 8640v1), or Faria N R, et al. 2020 (virological.org/t/genomic-characterisation-of-an-emergentsars-cov-2-lineage-in-manaus-preliminary-findings/586). The present invention encompasses amino acid sequences that are substantially homologous to amino acids sequences based on any one of the sequences identified in this application. The terms “sequence identity” and “sequence homology” are considered synonymous in this specification.

There are many established algorithms available to align two amino acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid sequences for comparison may be conducted, for example, by computer implemented algorithms (e.g., GAP, BESTFIT, FASTA or TFASTA), or BLAST and BLAST 2.0 algorithms.

In a comparison, the identity may exist over a region of the sequences that is at least 10 amino acid residues in length (e.g., at least 15, 20, 30, 40, 50, 75, 100, 150, 200, 250, 300, 350, 400, 450, 500, 550, 600, 650 or 685 amino acid residues in length, e.g. up to the entire length of the reference sequence). Each possibility represents a separate embodiment of the invention.

The antigens described herein are produced by an exogenous DNA sequence. The term “exogenous” as used herein refers to a polynucleotide or protein which is not naturally expressed within the fungus (e.g., heterologous polynucleotide from a different species). The exogenous polynucleotide may be introduced into the fungus in a stable or transient manner, so as to produce a ribonucleic acid (RNA) molecule and/or a polypeptide molecule.

The terms “DNA construct”, “expression vector”, “expression construct” and “expression cassette” are used to refer to an artificially assembled or isolated nucleic acid molecule which includes a nucleic acid sequence encoding a protein of interest and which is assembled such that the protein of interest is functionally expressed in a target host cell. An expression vector typically comprises appropriate regulatory sequences operably linked to the nucleic acid sequence encoding the protein of interest. An expression vector may further include a nucleic acid sequence encoding a selection marker.

The terms “polynucleotide”, “nucleic acid sequence”, and “nucleotide sequence” are used herein to refer to polymers of deoxyribonucleotides (DNA), ribonucleotides (RNA), and modified forms thereof in the form of a separate fragment or as a component of a larger construct. A nucleic acid sequence may be a coding sequence, i.e., a sequence that encodes for an end product in the cell, such as a protein.

A sequence (such as, nucleic acid sequence and amino acid sequence) that is “homologous” to a reference sequence refers herein to percent identity between the sequences, where the percent identity is at least 70%, at least 75%, preferably at least 80%, at least 85%, at least 90%, at least 95%, at least 98% at least 99% or at least 99.5%. Each possibility represents a separate embodiment of the present invention. Homologous nucleic acid sequences include variations related to codon usage and degeneration of the genetic code.

Nucleic acid sequences encoding the antigens of the present invention may be optimized for expression. Examples of such sequence modifications include, but are not limited to, an altered G/C content to more closely approach that typically found in filamentous fungi.

The phrase “codon optimization” refers to the selection of appropriate DNA nucleotides for use within a structural gene or fragment thereof that approaches codon usage within the organism of interest, and/or to a process of modifying a nucleic acid sequence for enhanced expression in the host cells of interest by replacing at least one codon (e.g., one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Therefore, an optimized gene or nucleic acid sequence refers to a gene in which the nucleotide sequence of a native or naturally occurring gene has been modified in order to utilize statistically-preferred or statistically-favored codons within the organism.

Sequence identity may be determined using a nucleotide/amino acid sequence comparison algorithm, as known in the art.

The term “coding sequence” is used herein to refer to a sequence of nucleotide starting with a start codon (ATG) containing any number of codons excluding stop codons, and a stop codon (TAA, TGA, TAA), which code for a functional polypeptide.

Any coding sequence, or amino acid sequence listed herein also encompasses truncated sequences, which are missing 1, 2, 3, 4, 5, 10, 15, 20, 25, 50, or more codons or amino acids from any part of the sequence. Truncated versions of coding sequences or amino sequences can be identified using nucleotide/amino acid sequence comparison algorithm, as known in the art.

Any coding sequence, or amino acid sequence listed herein also encompasses fused sequences, which contain besides the coding sequence provided herein, or a truncation of that sequence as defined above, other sequences. The fused sequences can be sequences as disclosed herein and other sequences. Fused coding sequences or amino sequences can be identified using nucleotide/amino acid sequence comparison algorithm, as known in the art.

DNA sequences are assembled to expression cassettes, selection cassettes and further to DNA constructs and/or expression vectors by conventional molecular biological approaches utilizing restriction endonucleases and ligases, Gibson assembly or yeast recombination. Also, the above can be synthesized by DNA synthesis service providers. As known in the art, several different techniques can achieve the same result.

DNA sequences are assembled to expression cassettes joining a 5′ regulatory regions (promoters), a coding sequence and a 3′ regulatory regions (terminators) as described hereinbelow and as are known in the art. Any combination of these three sequences can form a functional expression cassette.

The list of terminators includes, but are not limited to that of Th. heterothallica genes encoding for uncharacterized protein G2QF75 (XP_003664349); polyubiquitin homologue (G2QHM8, XP_003664133); uncharacterized protein (G2QIA5, XP_003664731); beta-glucosidase (G2QD93, XP_003662704); elongation factor 1-alpha (G2Q129, XP_003660173); chitinase (G2QDD4, XP_003663544) phosphoglycerate kinase (PGK) (Uniprot G2QLD8), glyceraldehyde 3-phosphate dehydrogenase (GPD) (G2QPQ8), phosphofructokinase (PFK) (G2Q605); or triose phosphate isomerase (TPI) (G2QBR0); actin (ACT) (G2Q7Q5); cbh1 (GenBank AX284115) or β-glucosidase 1 bgl1 (XM_003662656). Exogenous terminators include that of Aspergillus nidulans gpdA terminator.

5′ regulatory regions (promoters) are practically defined as a stretch of up to 2000 base pairs preceding the start codon of the coding sequence of the gene they regulate, provided that the preceding region is non-coding.

3′ regulatory regions (terminators) are practically defined as a stretch of up to 300 base pairs downstream from the end codon of the coding sequence of the gene, provided that the subsequent region is non-coding.

DNA sequences are also assembled to selection marker cassettes, which are expression cassettes where the coding sequence codes for a gene that provides a selective advantage when present in a transformed strain. Such advantage can be utilization of a new carbon or nitrogen source, a resistance to a toxic substance, etc.

Deletion of the proteases disclosed herein can be done as known in the art. In some embodiments, the deletion is performed by transformation of suitable DNA constructs. DNA constructs used for targeted transformation are composed of (a) a suitable vector that allows the maintenance of the DNA construct in a particular host, (b) zero, one or more expression cassettes in any direction, (c) a selection marker cassette in any direction and (d) sequences that are identical to select stretches of the target genomic DNA (also called as targeting arms). These components are placed so, that the two targeting arms encompass any expression cassettes and the selection marker cassette, so that when homologous recombination happens between the targeting arms and the two identical regions in the genomic DNA, the sequence between the targeting arms of the DNA constructs gets inserted into the chromosome, and replaces the sequence originally present on the chromosome. Using this principle, genes can be knocked out from, or inserted into the genome. By placing a sequence downstream of the selection marker cassette, which is identical to the sequence just upstream of the selection marker cassette, it is possible to recycle the marker as known in the art.

The term “regulatory sequences” refer to DNA sequences which control the expression (transcription) of coding sequences, such as promoters, enhancers and terminators.

The term “promoter” is directed to a regulatory DNA sequence which controls or directs the transcription of another DNA sequence in vivo or in vitro. Usually, the promoter is located in the 5′ region (that is, precedes, located upstream) of the transcribed sequence. Promoters may be derived in their entirety from a native source, or be composed of different elements derived from different promoters found in nature, or even comprise synthetic nucleotide segments. Promoters can be constitutive (i.e. promoter activation is not regulated by an inducing agent and hence rate of transcription is constant), or inducible (i.e., promoter activation is regulated by an inducing agent or environmental condition). Promoters may also restrict transcription to a certain developmental stage or to a certain morphologically distinct part of the organism. In most cases the exact boundaries of regulatory sequences have not been completely defined, and in some cases, cannot be completely defined, and thus DNA sequences of some variation may have identical promoter activity.

The term “terminator” is directed to another regulatory DNA sequence which regulates transcription termination. A terminator sequence is operably linked to the 3′ terminus of the nucleic acid sequence to be transcribed.

The terms “C1 promoter” and “C1 terminator” indicate promoter and terminator sequences suitable for use in C1, i.e., capable of directing gene expression in C1.

However, as known to the skilled artisan, the choice of promoters and terminators may not be critical, and similar results can be obtained with a variety of promoters and terminators providing similar or identical gene expression.

The term “operably linked” means that a selected nucleic acid sequence is in proximity with a regulatory element (promoter, enhancer and/or terminator) to allow the regulatory element to regulate expression of the selected nucleic acid sequence.

The present invention discloses the production of coronavirus antigens using genetically modified strains of Th. heterothallica C1. As described hereinabove, filamentous fungi of other species sharing endogenous similar pathways of precursor production can be also used.

According to certain embodiments, the polynucleotides of the present invention are designed based on the amino acid sequence of the antigen to be produced employing a codon usage of a filamentous fungus. According to certain embodiments, the filamentous fungus belongs to the group Pezizomycotina. According to some embodiments, the filamentous fungus belongs to a group selected from the group consisting of Sordariales, Hypocreales Onygenales, and Eurotiales including genera and species as described in the “definition” section hereinabove. According to certain exemplary embodiments, the fungus is Th. heterothallica. According to certain currently exemplary embodiments, the fungus is Th. heterothallica C1.

The DNA constructs or expression vector or plurality of same each comprises regulatory elements controlling the transcription of the polynucleotides within the at least one fungus cell. The regulatory element can be a regulatory element endogenous to the fungus, particularly to Th. heterothallica C1 or exogenous to the fungus.

According to certain embodiments, the regulatory element is selected from the group consisting of a 5′ regulatory element (collectively referred to as promoter), and 3′ regulatory element (collectively referred to as terminator), even though these nucleotide sequences may contain additional regulatory elements not classified as promoter or terminator sequences in the strict sense.

According to some embodiments, the antigens are expressed from polynucleotide within a DNA construct or expression vector that were transformed into the fungus. According to certain embodiments, the DNA construct or expression vector comprises at least one promoter operably linked to at least one polynucleotide containing a coding sequence, operably linked to at least one terminator. According to certain embodiments, the promoter is endogenous promoter of the fungus, particularly to Th. heterothallica. According to additional or alternative embodiments, the promoter is heterologous to the fungus, particularly to Th. heterothallica. According to certain embodiments, the terminator is endogenous terminator of the fungus, particularly to Th. heterothallica. According to additional or alternative embodiments, the terminator is heterologous to the fungus, particularly to Th. heterothallica.

According to certain exemplary embodiments, the DNA constructs contain synthetic regulatory elements called as “synthetic expression system” (SES) essentially as described in International (PCT) Application Publication No. WO 2017/144777.

According to certain embodiments, the polynucleotide is stably integrated into at least one chromosomal locus of the at least one cell of the genetically modified fungus. According to certain embodiments, the polynucleotide is stably integrated into a defined site on the fungal chromosomes. According to certain embodiments, the polynucleotide is stably integrated into a random site of the chromosome. According to certain embodiments, the polynucleotide may be incorporated in targeted or random fashion as 1, 2 or more copies to 1, 2 or more chromosomal loci. According to some embodiments, the at least two antigens are integrated to a same chromosomal locus. According to additional embodiments, the at least two antigens are integrated to different chromosomal loci.

According to certain exemplary embodiments, the present invention provides a genetically modified Th. heterothallica C1 fungus that enables producing at least two antigens produced from at least two transcription units. According to certain embodiments, the genetically modified Th. heterothallica C1 fungus comprises at least one cell having reduced expression and/or activity of KEX2 and/or ALP7 and at least one additional protease.

According to certain embodiments, a suitable medium for culturing the genetically modified fungi comprises a carbon source selected from the group consisting of glucose, sucrose, xylose, arabinose, galactose, fructose, lactose, cellobiose, and glycerol. According to some embodiments, the carbon source is provided from waste of ethanol production or other bioproduction from starch, sugar beet and sugar cane such as molasses comprising fermentable sugars, starch, lignocellulosic biomass comprising polymeric carbohydrates such as cellulose and hemicellulose.

According to certain currently exemplary embodiments, the fungus is Th. heterothallica C1. According to certain embodiments, the strain of Th. heterothallica C1 is selected from the group consisting of strain UV18-25, deposit No. VKM F-3631 D; strain NG7C-19, deposit No. VKM F-3633 D; and strain UV13-6, deposit no. VKM F-3632 D. Additional strains that may be used are HC strain UV18-100f deposit No. CBS141147; HC strain UV18-100f deposit No. CBS141143; LC strain W1L #100I deposit No. CBS141153; and LC strain W1L #100I deposit No. CBS141149 and derivatives thereof. Each possibility represents a separate embodiment of the present invention.

According to another aspect, the present invention provides a method for producing a fungus capable of producing two different viral antigens, the method comprising transforming at least one cell of the fungus with at least one polynucleotide encoding two different transcription units, said at least one cell of the fungus having reduced expression and/or activity of KEX2 and/or ALP7 and at least one additional protease.

The terms “reduced expression” or “inhibited expression” of a protein, in particular protease, are used interchangeably and include, but are not limited to, deleting or disrupting the gene that encodes for the protein.

The terms “reduced activity” or “inhibited activity” of a protein, in particular protease, are used interchangeably further include posttranslational modifications resulting in reduced or abolished activity of the protein.

Any method as is known in the art for transforming filamentous fungi with polynucleotide encoding for the viral antigens described herein can be used according to the teachings of the present invention.

According to yet another aspect, the present invention provides a method of producing at least two different viral antigens, the method comprising culturing the genetically modified fungus, particularly Th. heterothallica C1 fungi of the present invention in a suitable medium; and recovering the antigen products.

According to certain embodiments, the method comprises culturing genetically modified fungi as described herein, each expressing a different viral antigen. According to certain embodiments, the fungi express antigens of different coronavirus variants.

According to certain embodiments, the medium comprises a carbon source selected from the group consisting of glucose, sucrose, xylose, arabinose, galactose, fructose, lactose, cellobiose, and glycerol. According to certain embodiments the carbon source is waste obtained from ethanol production or other bioproduction from starch, sugar beet and sugar cane such as molasses comprising fermentable sugars, starch, lignocellulosic biomass comprising polymeric carbohydrates such as cellulose and hemicellulose.

According to some embodiment, the at least two antigens are purified from the fungal growth medium.

According to other embodiments, the at least two viral antigens are extracted from the fungal mass. Any method as is known in the art for extracting and purifying proteins from vegetative tissues can be used.

According to a further aspect, the present invention provides multiple viral antigens produced by the genetically modified fungus, particularly the genetically modified Th. heterothallica C1 of the present invention.

According to some embodiments, the at least two viral antigens are of a coronavirus antigen. According to some embodiments, at least one of the antigens is the full spike protein of coronavirus. According to certain embodiments, at least one of the antigens comprises the RBD sequence of the coronavirus spike protein, or a fragment thereof. According to certain embodiments, the RBD or fragment thereof is fuses, directly or indirectly, to Spytag. According to certain embodiments, the antigen is attached to a Spy catcher.

According to another aspect, the present invention provides a cultivation comprising a mix of a first and a second genetically modified filamentous fungus, each fungus comprising at least one cell capable of producing a viral antigen, wherein the second fungus comprising a cell capable of producing a viral antigen which is different from the viral antigen produced by the at least one cell of the first fungus.

According to some embodiments, the cultivation comprising a mix of at least three genetically modified filamentous fungi capable of producing at least three different antigens.

The fungi and the antigen are as described hereinabove. According to specific embodiments, the antigen is a coronavirus antigen. According to additional embodiments, the antigen is RBD.

According to some embodiments, each of the genetically modified filamentous fungus is capable of producing a different RBD variant.

According to some embodiments, the genetically modified filamentous fungus has reduced expression and/or protease activity of one or more proteases as described herein. According to certain embodiments, the genetically modified filamentous fungus has reduced expression and/or protease activity of ×13 or ×14 proteases.

According to some embodiments, the genetically modified filamentous fungus comprising at least one cell modified to produce an RBD antigen fused to a c-Tag, said cell has reduced expression and/or protease activity of ×13 or ×14 proteases.

According to another aspect, the present invention provides a genetically modified filamentous fungus comprising at least one cell modified to produce an RBD_of B.1.1.7-UK, RBD_B.1.351-SA, or RBD_1.1.28.1(P.1)-BR.

According to some embodiments, the immune-active molecules are antibodies.

The term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g., scFv or sFv); and multispecific antibodies formed from antibody fragments. In particular embodiments, the antibodies are single-chain antibody fragments comprising a variable heavy chain region and/or a variable light chain region, such as scFvs.

According to some embodiments, the antibody is selected from the group consisting of Atezolizumab (Tecentriq), Avelumab (Bavencio), Dostarlizumab (Jemperli), Durvalumab (Imfinzi), Ipilimumab (Yervoy), Nivolumab (Opdivo), Pembrolizumab (Keytruda).

According to some embodiments, the polynucleotides encoding the immune-active molecules or proteins are inserted to specific sites in the fungal genome using the CRISPR system.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Expression of Wuhan SARS-Cov-2 RBD with the Ctag in Protease Deficient C1 Strain

Receptor binding domain (RBD) of Wuhan SARS-CoV-2 spike protein was expressed in protease deficient C1 strain with the C-terminal C-tag. The construct contained a sequence coding for a C1 endogenous CBH1 signal sequence, the residues 333-527 of the Spike protein from SARS-CoV-2, a Gly-Ser-linker and the C-tag flanked by recombination sequences to the C1 expression vector and MssI restriction enzyme sites. The fragment was synthetized by GenScript (USA). Amino acid sequence is set forth in SEQ ID NO: 10, and DNA sequence in SEQ ID NO: 11. The codon usage of the gene was optimized for expression in Thermothelomyces heterothallica. The synthetized fragment was amplified by PCR from the GenScript plasmid and cloned by Gibson Assembly (NEBuilder® HiFi DNA Assembly Cloning Kit, New England Biolabs) method into the Pac site of the C1 expression vector pMYT1055 between endogenous C1 bgl8 promoter and C1 chi1 terminator. The correct sequence of the construct was confirmed by sequencing the fragment inserted into the plasmid. A plasmid of correct sequence was given the plasmid number pMYT1142 (SEQ ID NO: 12).

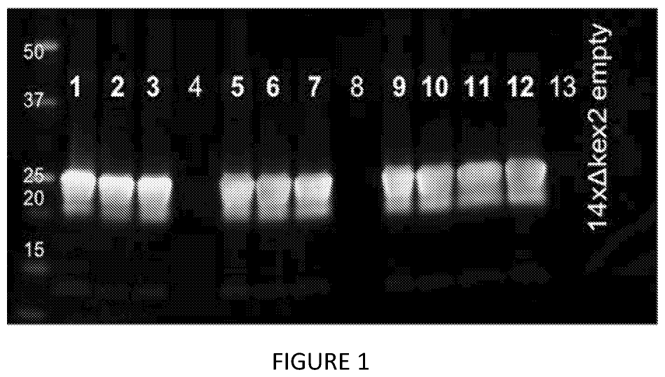

Expression vector pMYT1142 and a mock vector pMYT1140 (SEQ ID NO: 13), which is needed for completion of the hygromycin resistance marker gene and integration to the bgl8 locus, were digested with MssI and transformed to the DNL155 strain from which fourteen protease genes have been deleted. The transformation was done with protoplast/PEG method as described before (WO/2021/094935) and transformants were selected for nia1+ phenotype and hygromycin resistance. Transformants were streaked onto selective medium plates and inoculated from the streaks to liquid cultures in 24-well plates. The medium components were (in g/L) glucose 5, yeast extract 1, (NH₄)2SO₄ 4.6, MgSO₄·7H₂O 0.49, KH₂PO₄ 7.48, and (in mg/L) EDTA 45, ZnSO₄·7H₂O 19.8, MnSO₄·4H₂O 3.87, CoCl₂·6H₂O 1.44, CuSO₄·5H₂O 1.44, Na₂MoO₄·2H₂O 1.35, FeSO₄·7H₂O 4.5, H₃BO₄ 9.9, D-biotin 0.004, 50 U/ml Penicillin and 0.05 mg Streptomycin. The 24-well plates were incubated at 35° C. with 800 RPM shaking for four days. Culture supernatants were collected and analysed by Western blotting performed with standard methods with the primary detection agent Capture Select Biotin Anti-C-tag conjugate (ThermoFisher) and the secondary agent IRDye 800CW Streptavidin (Li-Cor). Western analysis (FIG. 1) showed that a strong signal of the expected size for a number transformants was detected, showing that Wuhan-RBD-C-tag was produced in C1.

Transformants producing the RBD-C-tag protein were purified by single colony plating, and purified clones were verified by PCR for correct integration of the expression cassette and for loss of the bgl8 gene. Mycelium from the transformant streaks was dissolved in 20 mM NaOH and incubated at 100° C. to lyse the cells. 1-2 μl of this solution was used as template for PCR with Phire Plant PCR kit™ (Thermo Fisher). 5′ integration of the expression cassette in the bgl8 locus was shown by a PCR reaction with the primers set forth as SEQ ID NOs: 40 (oMYT0746) and 42 (oMYT2823) and 3′ integration of the expression cassette in the bgl8 locus was shown by a PCR reaction with the primers set forth as SEQ ID NOs: 43 (oMYT1277) and 44 (oMYT0748). The loss of bgl8 ORF was shown by a PCR reaction with the primers set forth as SEQ ID NOs: 45 (oMYT0529) and 47 (oMYT0649). Transformants positive for correct integration and negative for bgl8 ORF were further analyzed by quantitative PCR with the primers set forth as SEQ ID NOs: 48 (oMYT0648) and 47 (oMYT0649) to demonstrate that tested clones have totally lost the bgl8 gene and are pure clones. One verified clone producing RBD-C-tag was stored at −80° C. and given the strain number M4169. The final verification of the M4169 strain was carried out by PCR amplification the RDB-C-tag fragment from the M4169 strain with the primers set forth as SEQ ID NOs: 49 (oMYT0086) and 50 (oMYT0107). Sequencing result of the amplified fragment confirmed the correct sequence of RBD-C-tag in the M4169 strain.

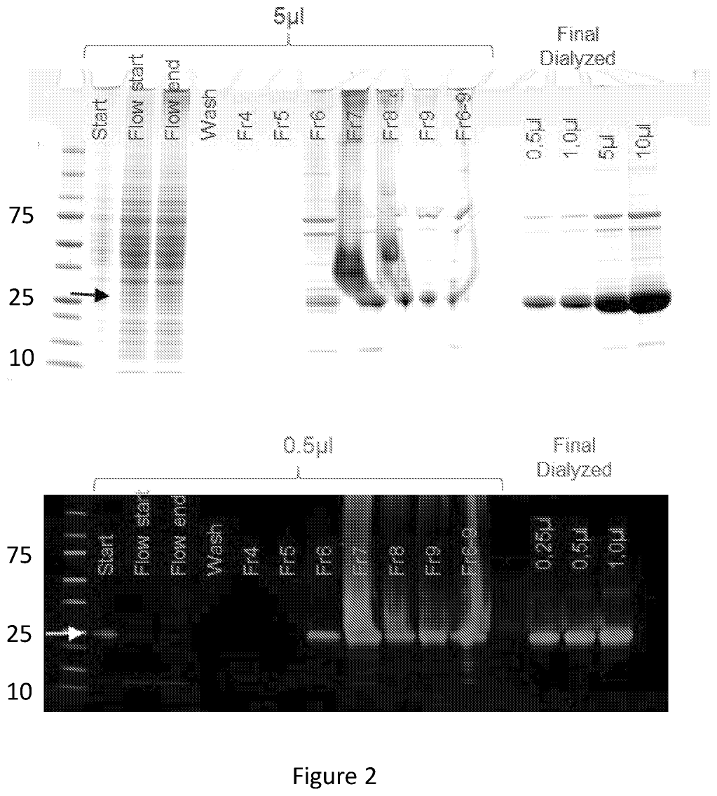

The C1 strain M4169 producing RBD-C-tag protein was cultivated in 1 and 2 L bioreactor in a fed-batch process in a medium with yeast extract as an organic nitrogen source and glucose as a carbon source. The cultures were performed at 38° C. for five to seven days. After ending the cultivation, mycelia were removed by centrifugation at 4000 g for 20 minutes, phenylmethylsulfonyl fluoride (PMSF) was added in 1-2 mM concentration to inhibit protease activity in the obtained culture supernatant and the supernatant was stored at −80° C. For protein purification, two affinity chromatography methods were used. First, RBD-C-tag was purified using CaptureSelect™ C-tag resin (Thermo Scientific). Purification was first performed in small-scale using ÄKTA Start protein purification system (Cytiva) operated with a flow rate of 1 ml/min and prepacked 1 ml CaptureSelect C-tag XL column. A 2 ml sample of bioreactor liquid culture was thawed on ice followed by centrifugation 2×10 min 13500 g at +4° to obtain a clarified sample. Clarified sample of 0.5 ml was diluted in 2 ml 1×PBS (12 mM Na₂HPO₄*2 H₂O, 3 mM NaH₂PO₄*H₂O, 150 mM NaCl pH 7.3). Column was first equilibrated with 5 column volumes (CV) of 1×PBS prior to applying the diluted sample via loop. After sample loading, the column was washed with 15CV of 1×PBS and then eluted with one-step gradient of 5CV of 20 mM Tris-HCl, 2M MgCl₂, 1 mM EDTA pH7.5 with fraction volume of 1 ml. The quantity of the eluted RBD-C-tag was quantified by integrating the UV trace of the elution peak with the Unicorn 1.0 software included in the ÄKTA Start system. The extinction coefficient of 1.498 was used in calculating RBD-C-tag amount. After elution, the column was regenerated with 5CV of 0.1M glycine pH 2.3 and washed with 1×PBS until pH7.3 was reached. For a larger scale purification, 100 ml of liquid culture was thawed on ice, and after thawing the sample was clarified by centrifugation 3×20 min 20000 g at +4° C. followed by filtration through a 0.45 μm nylon filter. 90 ml of the clear supernatant was diluted with 1×PBS to final volume of 200 ml. The C-tag affinity purification was performed with a 10 ml column of packed CaptureSelect C-tagXL resin attached to the ÄKTA Start protein purification system (Cytiva) and operated with a flow rate of 2.5 ml/min. Column was first equilibrated with 5CV of 1×PBS prior loading the sample. After sample loading, the column was washed with 15 CV of 1×PBS and then eluted with one-step gradient of 5CV of 20 mM Tris-HCl, 2M MgCl₂, 1 mM EDTA pH7.5 with fraction volume of 3 ml. The column was regenerated as in small-scale C-tag affinity purification. The quantity of the eluted RBD was quantified as in small scale C-tag affinity purification. Elution fractions containing the protein were pooled for dialysis step to exchange the elution buffer to 1×PBS. Pooled fractions were packed in a 12-15 ml dialysis cassette and the dialysis was done in 1.5 l in 1×PBS for 1 h at +4° C. with stirring on a magnetic stirrer. 1×PBS was changed to fresh buffer after 1 h and dialysis was continued for 2 h in the same conditions. Finally, fresh 1×PBS was changed and dialysis was continued overnight. Concentration of dialyzed RBD was determined with the Nanodrop spectrophotometer measuring absorbance at 280 nm and using extinction coefficient of 1.498 for RBD-C-tag. Aliquots of RBD-C-tag preparates were stored at −80° C. C-tag affinity purification of RBD-C-tag from M4169 fermentation is shown as an example in FIG. 2. SARS-CoV-2 Spike RBD Antibody, Rabbit polyclonal antiserum (SinoBiologicals) was used as primary antibody in Western detection and secondary detection agent was Goat anti-rabbit IRDye 680RD (Li-Cor).

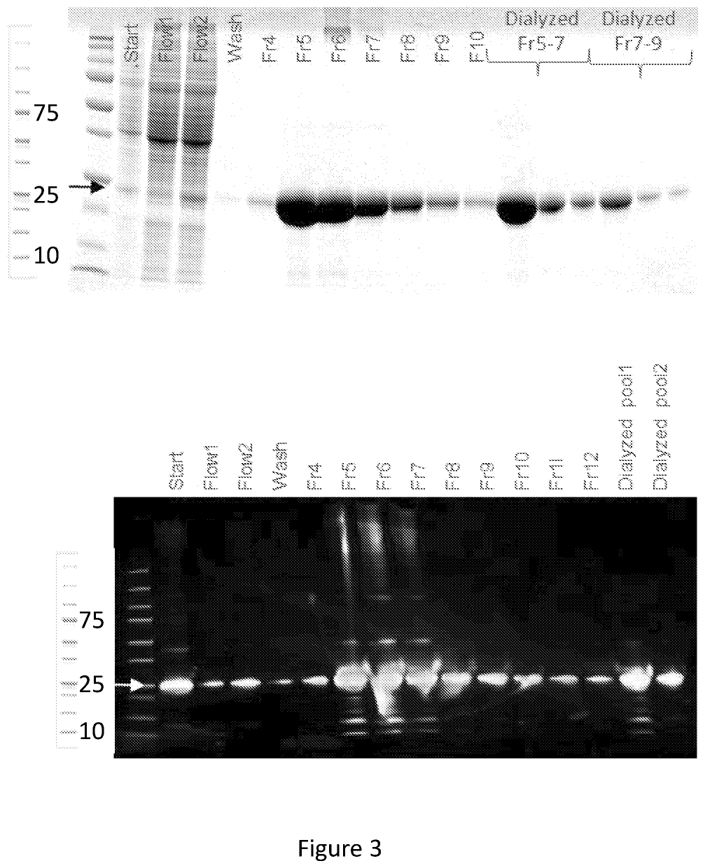

Wuhan-RBD-C-tag was also purified using Repligen prototype CoV-2 Spike Protein affinity resin version 1 (Repligen v1). Similar to the C-tag affinity purification, small-scale purification was performed with 1 ml column and with ÄKTA Start protein purification system, operated in a flow rate of 0.5 ml/min. Sample clarification and dilution in 1×PBS was performed in similar manner as for C-tag affinity purification. Column was first equilibrated with 5 CV of 1×PBS prior to applying the diluted sample via loop. After sample loading, the column was washed with 15 CV of 1×PBS and then eluted with one-step gradient of 5 CV of 50 mM NaAc 30 mM NaCl pH 3.5 with fraction volume of 1 ml. After elution, pH in elution fractions was adjusted with 1M Tris-HCl pH9.5 to pH 7. The quantity of the eluted RBD-C-tag was quantified as in C-tag affinity purification. A larger scale purification was performed with 10 ml column of packed Repligen v1 resin attached to ÄKTA Start protein purification system and operated with a flow rate of 5.0 ml/min. 200 ml of fermentation supernatant was clarified as in C-tag affinity purification in larger scale. The sample was diluted with 200 ml of 1×PBS to final volume of 400 ml. Column was first equilibrated with 5 CV of 1×PBS prior to loading the sample. After sample loading, the column was washed with 15CV 1×PBS and eluted with 5CV of 50 mM NaAc+30 mM NaCl pH 3.5 with fraction volume of 3 ml. After elution, pH in elution fractions were adjusted with 1M Tris-HCl pH 9.5 to pH 7. Column was regenerated with 5 CV of 200 mM Acetic acid (pH 2.5) and washed with 1×PBS until pH 7.3 was reached. The amount of the eluted Wuhan RBD-C-tag was quantified as above with C-tag affinity purification. Elution fractions containing majority of the protein were pooled to exchange the elution buffer to 1×PBS buffer using dialysis as described above with C-tag affinity purification. Concentration of dialyzed RBD-C-tag was determined as above with C-tag affinity purification and aliquots of Wuhan RBD-C-tag were stored at −80° C. Affinity purification of Wuhan RBD-C-tag by Repligen v1 from M4169 fermentation is shown as an example in FIG. 3. SARS-CoV-2 Spike RBD Antibody, Rabbit polyclonal antiserum (SinoBiologicals) was used as primary antibody in Western detection and secondary detection agent was Goat anti-rabbit IRDye 680RD (Li-Cor).

### Example 2: Expression of Wuhan SARS-CoV-2 RBD with C-Tag from Two Expression Cassettes in Protease Deficient C1 Strain

To increase the production level of Wuhan-RBD-C-tag, a C1 strain harboring two expression cassette copies was constructed. Both gene copies have the same sequence as in the single copy RBD-C-tag strain M4169 in Example 1. Two plasmid constructs (5′arm and 3′arm) are needed for this strategy, both harbouring one expression cassette. In C1 cells, the recombination between the selection marker fragments within 5′arm and 3′arm plasmids makes the marker gene functional and enables the transformants to grow under selection. Orientation of gene copies in the genomic locus is such that one gene copy is in forward direction while the second gene copy is in reverse direction. The same bgl8 locus was used for expression as in the single copy strain M4169 enabling use of the same 5′arm plasmid pMYT1142 that was used in construction of the single copy strain M4169. The 3′arm plasmid harbouring additional gene copy was constructed by PCR amplification of RBD-C-tag fragment using pMYT1142 as a template and cloning by Gibson Assembly (NEBuilder® HiFi DNA Assembly Cloning Kit, New England Biolabs) method into the Pac site of the C1 expression vector pMYT1056 between endogenous C1 bgl8 promoter and C1 bgl8 terminator. The plasmid of correct sequence was given the plasmid number pMYT1366 (SEQ ID NO: 14).

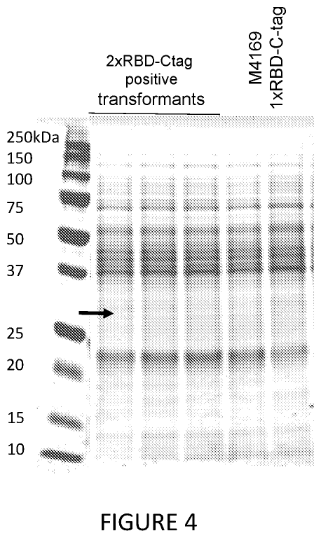

For the double copy expression, both 5′arm and 3′arm plasmids were digested with MssI and transformed to the DNL159 strain from which fourteen protease genes have been deleted. The transformation and 24-well screening cultivation were performed as described for construction of the single copy strain M4169. FIG. 4 shows an example of stained SDS-PAGE of 24-well plate culture samples of correct transformants harboring two copies of RBD-C-tag. Compared to the M4169 strain with one copy of RBD-C-tag, the production level is increased in the two copy clones. Transformants producing the Wuhan-RBD-C-tag protein were purified by single colony plating, and verified by standard PCR for correct integration of the expression cassette and by qPCR for clone purity as in Example 1. Additionally, the presence of two RBD gene copies was shown by two PCR reactions with the primers set forth as SEQ ID NOs: 49 (oMYT0086) and 50 (oMYT0107) for one gene copy and as SEQ ID NOs: 49 (oMYT0086) and 51 (oMYT0089) for the other gene copy. A verified clone was stored at −80° C. and given the strain numbers M4937.

The C1 strain M4937 was cultivated in 1 L bioreactor in a fed-batch process as for the M4169 strain and RBD-C-tag was purified from supernatant samples in small scale with C-tag affinity and with Repligen v1 affinity methods as for the M4169 strain. As a result, the purification yield of RBD-C-tag from the M4937 strain harboring two gene copies was 1.2-1.3× higher as compared to the purification yield from M4169 with the same purification method (data not shown).

### Example 3: Expression of Wuhan SARS-CoV-2 RBD without a Tag in Protease Deficient C1 Strain

Receptor binding domain (RBD) of Wuhan SARS-CoV-2 spike protein was also expressed in protease deficient C1 strain without C-terminal C-tag. Amino acid sequence is set forth in SEQ ID NO: 15, and DNA sequence in SEQ ID NO: 16. The GenScript synthetized plasmid used in constructing pMYT1142 was used as a template in PCR amplification with oligos which omitted the Gly-Ser linker and C-tag resulting in fragment of CBH1 signal sequence and RBD flanked with recombination sites to the expression vector. The fragment was cloned by Gibson Assembly (NEBuilder® HiFi DNA Assembly Cloning Kit, New England Biolabs) method into the PacI site of the C1 expression vector pMYT1055 between the endogenous C1 bgl8 promoter and C1 chi1 terminator. A plasmid of correct sequence was given the plasmid number pMYT1237 (SEQ ID NO: 17).

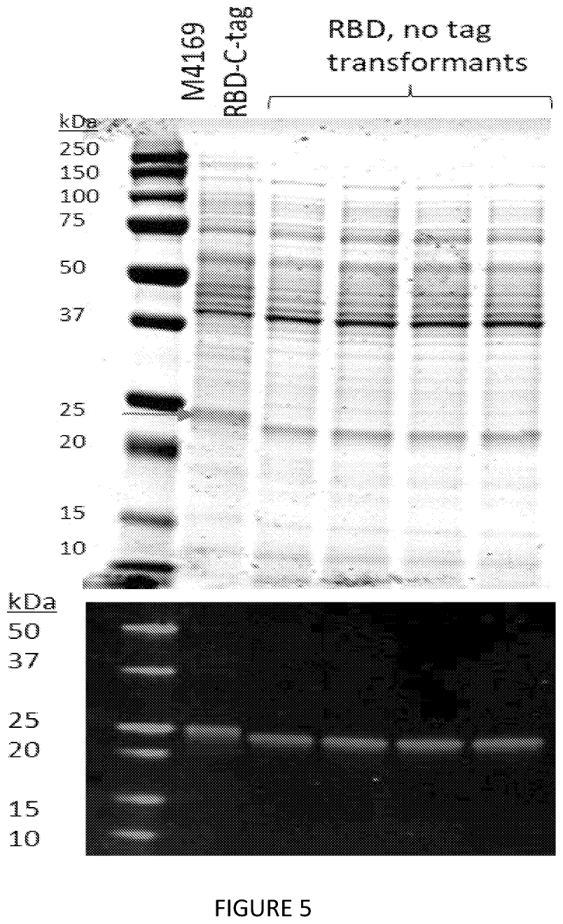

Expression vector pMYT1237 and a mock vector partner pMYT1140 were co-transformed to the DNL155 strain from which fourteen protease genes have been deleted as generation of the M4169 strain. Transformant screening, single colony purification and PCR screening were also performed as in generation of the M4169 RBD-C-tag strain in Example 1 except that culture supernatants were analyzed by Western blotting with SARS-CoV-2 (2019-nCoV) Spike RBD Antibody, Rabbit polyclonal antiserum (SinoBiologicals) and the secondary detection agent was Goat anti-rabbit IRDye 680RD (Li-Cor). A verified clone was stored at −80° C. and given the strain number M4393. Western analysis (FIG. 5) showed that a signal of the expected size for a number transformants was detected, showing that Wuhan-RBD without any tag was produced in C1, yet in somewhat lower level than the RBD with C-tag.

The C1 strain M4393 was cultivated in 1 L bioreactor in a fed-batch process as for the M4169 strain and RBD was purified from supernatant samples in small scale with Repligen v1 affinity method as described for the M4169 strain. A preparate with similar purity as for RBD-C-tag was obtained (data not shown).

### Example 4: Expression of Three SARS-CoV-2 RBD Variants Separately in Protease Deficient C1 Strain

Three variants of Receptor Binding Domain (RBD) of SARS-CoV-2 spike protein were expressed in the protease deficient C1 strain DNL155. The three variants are: 1) RBD_B.1.1.7-UK (alpha-UK) having N501Y mutation, amino acid sequence is set forth in SEQ ID NO: 18 (including C1 cbh1 signal sequence, GSG-linker and C-tag), and DNA sequence in SEQ ID NO: 19; 2) RBD_B.1.351-SA (beta-SA) having K417N, E484K and N501Y mutations, amino acid sequence is set forth in SEQ ID NO: 22 (including C1 cbh1 signal sequence, GSG-linker and C-tag), and DNA sequence in SEQ ID NO: 23; and 3) RBD_1.1.28.1(P.1)-BR (gamma-BR) having K417T, E484K and N501Y mutations, amino acid sequence is set forth in SEQ ID NO: 26 (including C1 cbh1 signal sequence, GSG-linker and C-tag), and DNA sequence in SEQ ID NO: 27. Each variant contained the residues 333-527 of the Spike protein from SARS-CoV-2, a Gly-Ser-linker and the C-tag. The fragment of each variant was synthesized by GenScript (USA) and the optimized sequence of Wuhan RBD (in pMYT1142, Example 1) was used as the basis from which the mutated amino acids were replaced with the codon most frequent in C1. The synthetized fragment design was similar to the Wuhan RBD with C-tag as in Example 1 except that the Gly/Ser-linker between the RBD variant and the C-tag was three amino acids long whereas in Wuhan RBD-C-tag the linker was five amino acids long. Variant RBDs were expressed from a construct having two expression cassettes in the same locus. Two plasmids (5′arm and 3′arm), both harbouring one expression cassette, were made for each variant. In C1 cells, the recombination between the selection marker fragments within 5′arm and 3′arm plasmids makes the marker gene functional and enables the transformants to grow under selection. Similar to the Wuhan-RBD-C-tag strain of two copies, the orientation in the genomic locus is such that one gene copy is in forward direction while the second gene copy is in reverse direction. For the 5′arm plasmids, synthesized fragments were amplified by PCR from the GenScript plasmids and cloned by Gibson Assembly (NEBuilder® HiFi DNA Assembly Cloning Kit, New England Biolabs) method into the PacI site of the C1 expression vector pMYT1055 between endogenous C1 bgl8 promoter and C1 chi1 terminator. The correct sequences of the constructs were confirmed by sequencing the fragments inserted into the plasmids. Plasmids of correct sequence were given the plasmid numbers pMYT1572 (SEQ ID NO: 20) for alpha-UK, pMYT1574 (SEQ ID NO: 24) for beta-SA and pMYT1576 (SEQ ID NO: 28) for gamma-BR, respectively. For the 3′arm plasmids, synthesized fragments in GenScript plasmids were cut out with MssI restriction enzyme and cloned by Gibson Assembly (NEBuilder® HiFi DNA Assembly Cloning Kit, New England Biolabs) method into the PacI site of the C1 expression vector pMYT1056 between endogenous C1 bgl8 promoter and C1 bgl8 terminator. Plasmids of correct sequence were given the plasmid numbers pMYT1573 (SEQ ID NO: 21) for alpha-UK, pMYT1575 (SEQ ID NO: 25) for beta-SA and pMYT1577 (SEQ ID NO: 29) for gamma-BR, respectively.

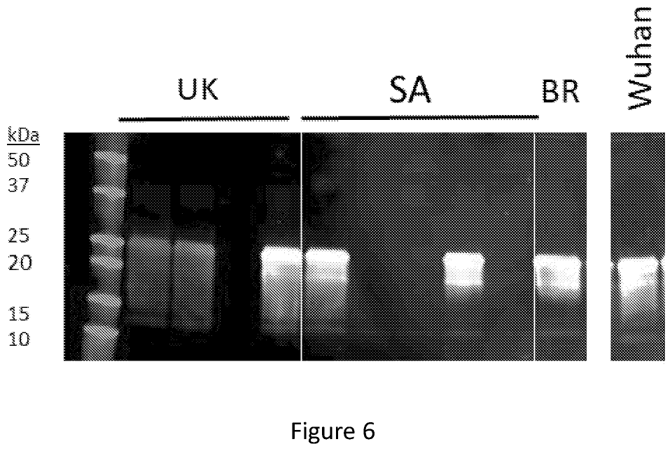

For double copy expression, both 5′arm and 3′arm plasmids were digested with MssI and plasmids harbouring the same variant gene were transformed with protoplast method (reference: C1 glycoengineering patent) to the DNL155 strain from which fourteen protease genes have been deleted. The transformation and screening of transformants by 24-well cultivation was performed as in Example 1 except that culture supernatants were analysed by Western blotting with two primary detection agents simultaneously: SARS-CoV-2 (2019-nCoV) Spike RBD Antibody, Rabbit polyclonal antiserum (SinoBiologicals) and Capture Select Biotin Anti-C-tag conjugate (ThermoFisher). The secondary detection agents were Goat anti-rabbit IRDye 680RD (Li-Cor) and IRDye 800CW Streptavidin (Li-Cor). FIG. 6 shows an example of Western blotting result with at least one positive transformant for each RBD variant. Strong signals of the expected size with both primary antibodies were detected and production levels of the variant RBD-C-tag proteins appeared to be equal to the M4169 control strain producing Wuhan RBD-C-tag. Transformants producing the RBD-C-tag variant proteins were purified by single colony plating, and purified clones were verified by PCR for correct integration of the expression cassette and by qPCR for clone purity as in Example 1 except the 3′ integration of the expression cassette in the bgl8 locus was shown by a PCR reaction with the primers set forth as SEQ ID NOs: 42 (oMYT2823) and 44 (oMYT0748). Presence of two RBD genes were verified by PCR as in Example 2. qPCR results showed that the transformants analysed by qPCR were actually pure prior to single colony plating purification. One verified transformant for each variant producing alpha-UK, beta-SA and gamma-BR were stored at −80° C. and given the strain numbers M5260, M5266 and M5270, respectively. For final confirmation of the production strains, both genomic RBD-C-tag copies were PCR amplified from M5260, M5266 and M5270 strains and the obtained fragments were sequenced. Primer used in PCR amplification and sequencing set forth as SEQ ID NOs: 49 (oMYT0086) and 50 (oMYT0107) for one gene copy and as SEQ ID NOs: 49 (oMYT0086) and 51 (oMYT0089) for the other gene copy. Sequencing results confirmed the correct sequence of RBD-C-tag in all three strains.

The C1 strains M5260 (alpha-UK), M5266 (beta-SA) and M5270 (gamma-BR) were cultivated in 1 L or 2 L bioreactors in a fed-batch process as described in Example 1. All variants were purified by C-tag affinity in small scale as in Example 1. Alpha-UK variant from M5260 was purified in small and larger scale with Repligen v1 resin as described in Example 1. For beta-SA and gamma-BR, Repligen prototype CoV-2 Spike Protein affinity resin version 2 (Repligen v2) was used in small and larger scale purification since Repligen v21 does not bind efficiently beta-SA or gamma-BR. Small-scale purification procedure of Repligen v2 was similar to Repligen v1. Purification procedure in larger scale with Repligen v2 had a few differences as compared with the Repligen v1 procedure. Fermentation culture supernatant (100-200 ml) was diluted 1:3 in 1×PBS pH 7.8 and pH of sample was adjusted to pH 7.8 prior to sample loading into the column. Additionally, column was washed with same buffers as for Repligen v1 but buffers were adjusted to pH 7.8. Extinction coefficients used were 1.568, 1.569 and 1.570 for alpha-UK, beta-SA and gamma-BR, respectively. Samples from all purifications were analyzed in stained SDS-PAGE and with Western blotting using the same detection as above in transformant screening. This showed that the purification results of the three RBD-C-tag variants were similar to those from Wuhan-RBD-C-tag and resulted in purified proteins of similar purity level as Wuhan-RBD-C-tag (FIG. 3).

### Example 5: Expression of Two SARS-CoV-2 Variants Separately in Protease Deficient C1 Strain

Two variants of Receptor Binding Domain (RBD) of SARS-CoV-2 spike protein were expressed in the protease deficient C1 strain DNL155 as single-copy expression with and without C-tag. The two variants are: 1) RBD_B.1.617.2 (Delta) having L452R and T478K mutations and 2) RBD_B.1.351-SA (beta-SA) having K417N, E484K and N501Y mutations. The constructs contained a sequence coding for a C1 endogenous CBH1 signal sequence, the residues 333-527 of the Spike protein from SARS-CoV-2, and the versions with C-tag also contained a Gly-Ser-linker and the C-tag (EPEA). The amino acid sequence of RBD-Delta (without C-tag) set forth as SEQ ID NO: 31 and the corresponding DNA sequence as SEQ ID NO: 32. The amino acid sequence of RBD-Delta-C-tag set forth as SEQ ID NO: 33 and the corresponding DNA sequence as SEQ ID NO: 34. The amino acid sequence of RBD-beta-SA (without C-tag) set forth as SEQ ID NO: 37 and the corresponding DNA sequence as SEQ ID NO: 38. The amino acid and DNA sequences of RBD-beta-SA-C-tag are the same as described in the Example 4 (SEQ ID NO: 22 and SEQ ID NO: 23). The RBD-Delta fragment without C-tag was synthetized by GenScript (USA). The codon usage of the gene was optimized for expression in Thermothelomyces heterothallica. The fragment contained also the C1 CBH1 signal sequence, was flanked by recombination sequences to the C1 expression vector and had MssT restriction enzyme sites outside the flanking sequences for release of the fragment from the plasmid. The RBD-beta-SA-C-tag is described in the Example 4. The synthetized fragments were either released from the GenScript plasmid using MssI restriction enzyme or produced by PCR with reverse primer containing GSG-linker and C-tag for Delta or with primer removing the linker and C-tag for beta-SA, respectively. The cloning into the C1 expression vector pMYT1055 between endogenous C1 bgl8 promoter and C1 chi1 terminator was made as described in the Example 4. The correct sequences for the expression plasmids were confirmed by sequencing the fragment regions inserted into the plasmids. The plasmids with correct sequences were given the plasmid numbers pMYT1717 (RBD-Delta without C-tag, SEQ ID NO: 35), pMYT1716 (RBD-Delta-C-tag, SEQ ID NO: 36) and pMYT1715 (RBD-beta-SA without C-tag, SEQ ID NO: 39). The expression plasmid pMYT1574 for RBD-beta-SA-C-tag is described in the Example 4 (SEQ ID NO: 24).

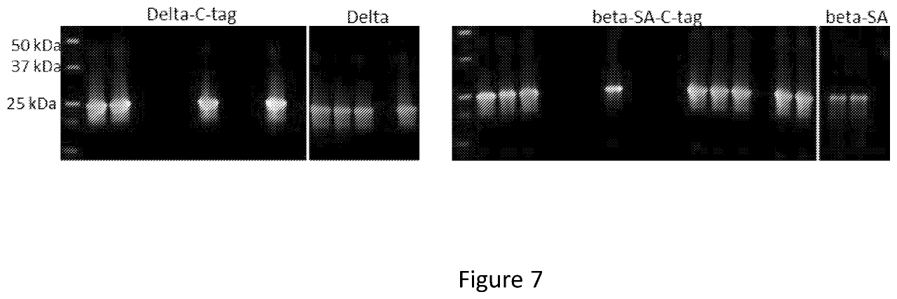

Variant RBDs were expressed in C1 strain DNL155 from which fourteen protease genes have been deleted. For the transformations the expression vectors pMYT1715, pMYT1716 and pMYT1717 and a mock vector pMYT1140 (SEQ ID NO: 13), which is needed for completion of the hygromycin resistance marker gene and integration to the bgl8 locus, were digested with MssI and co-transformed to the DNL155 strain as pairs (pMYT1715+pMYT1140, pMYT1716+pMYT1140 and pMYT1717+pMYT1140). The transformations and cultivation in 24-well plates were carried out essentially as described above. The culture supernatants were analyzed by Western blotting with two primary detection agents simultaneously as described in the Example 4. FIG. 7 shows an example of Western blotting result with at least two positive transformants for all four single-copy RBD variant versions. Clear signals of the expected size are detected both with anti-RBD and anti-C-tag antibodies (if applicable).

A few transformants producing each of the variant proteins were verified by PCR for correct integration of the expression cassette and loss of the bgl8 ORF using following primers: 5′ integration of the expression cassettes in the bgl8 locus was shown by a PCR reaction with the primers set forth as SEQ ID NOs: 41 (oMYT0606) and 42 (oMYT2823), 3′ integration of the expression cassette in the bgl8 locus was shown by a PCR reaction with the primers set forth as SEQ ID NOs: 43 (oMYT1277) and 44 (oMYT0748) and the loss of bgl8 ORF was shown by a PCR reaction with the primers set forth as SEQ ID NOs: 45 (oMYT0529) and 46 (oMYT0532)/47 (oMYT0649). The selected transformants were further purified by single colony plating. Purified clones were verified by repeating the PCR for correct integration of the expression cassette (with the primers above) and by qPCR for clone purity. qPCR was carried out essentially as described in Example 1. The results indicated that the transformants analyzed by qPCR were often pure already prior to the purification using single colony plating. One verified transformant from each variant producing RBD-Delta, RBD-Delta-C-tag, RBD-beta-SA or RBD-beta-SA-C-tag were stored at −80° C. as M5517, M5577, M5514 and M5578, respectively.

The C1 strains M5517 (RBD-Delta), M5577 (RBD-Delta-C-tag), M5514 (RBD-beta-SA) and M5578 (RBD-beta-SA-C-tag) were cultivated in 1 L or 2 L bioreactors in a fed-batch process for five days as described in Example 1. All variants were purified in small-scale with Repligen resin; RBD-Delta versions with v1 and RBD-beta-SA versions with v2, respectively. The variants with C-tag were also purified by C-tag affinity in small scale. Procedures for small-scale purifications using these resins have been described in Examples 1 and 4. The main difference to the previous descriptions was that for these purifications the system was operated with a flow rate of 0.5 ml/min (except priming steps with 1.0 ml/min). Small-scale affinity purifications from fermentation samples of RBD-Delta (M5517) using Repligen resin and of RBD-Delta-C-tag (M5577) and RBD-beta-SA-C-tag (M5578) using both resins resulted in similar purity level as Wuhan-RBD-C-tag (data not shown). Purifications of single-copy beta-SA-C-tag indicate a lower production level for this variant compared to other variants.

RBD-Delta variant from M5517 and RBD-Delta-C-tag variant from M5577 were also purified in larger scale using 10 ml column packed with Repligen v1 resin with small modifications to the procedure described in Example 1. For RBD-Delta (M5517) purification, over 100 ml of fermentation supernatant was thawed on ice overnight. Supernatant was clarified by centrifugation 2×20 min, 20000 g at +4° C. followed by filtration through a 0.45 μM Nylon filter and pH adjustment to 7.3. 100 ml of the cleared supernatant was diluted with three volumes of 1×PBS (12 mM Na₂HPO₄*2 H₂O, 3 mM NaH₂PO₄*H₂O, 150 mM NaCl pH 7.3) to final volume of 400 ml. The ÄKTA Pure protein purification system (Cytiva) was operated with a flow rate of 2.5 ml/min during sample application and elution, otherwise with 5.0 ml/min. Column was first equilibrated with 5 column volumes (CV) of 1×PBS prior to loading the sample. After sample loading, the column was washed in three steps (5CV of 1×PBS pH 7.3, 5CV of 1×PBS+0.5 M NaCl pH 7.3, 8CV of 1×PBS pH 7.3) and then eluted with isocratic elution using 7CV of 100 mM NaAc+30 mM NaCl pH 3.5 with fraction volume of 1.6 ml. The fractions contained additional 0.4 ml of 1 M Tris-HCl pH 9.0 for initial pH adjustment. The amount of the eluted RBD-Delta was quantified by integrating the UV trace of the elution peak with the Unicorn 1.0 software included in the ÄKTA Pure system. The extinction coefficient of 1.53 was used in calculating the amount of RBD-Delta. Elution fractions containing majority of the protein were pooled to exchange the elution buffer to 1×PBS buffer using dialysis. Pooled fractions were packed in a 15 ml dialysis cassette and dialyzed in 3 l in 1×PBS for 1 h at +4° C. with stirring on a magnetic stirrer. 1×PBS was changed to fresh buffer after 1 h and dialysis was continued for 2 h using the same conditions. Finally, fresh 1×PBS was changed and dialysis was continued overnight. Concentration of dialyzed RBD-Delta was determined with a Nanodrop spectrophotometer measuring absorbance at 280 nm and using extinction coefficient 1.53. Aliquots of RBD-Delta were stored at −80° C. Purification of RBD-Delta-C-tag (M5577) was carried out essentially as described for RBD-Delta above using 10 ml Repligen v1 resin and ÄKTA Pure system. 85 ml of supernatant and pH 7.8 for PBS-buffers were used in the purification. The extinction coefficient of 1.49 was used in calculating the amount of RBD-Delta-C-tag obtained from the purification. Dialysis of the RBD-Delta-C-tag was carried out essentially as described above for RBD-Delta. Aliquots of RBD-Delta-C-tag were stored at −80° C.

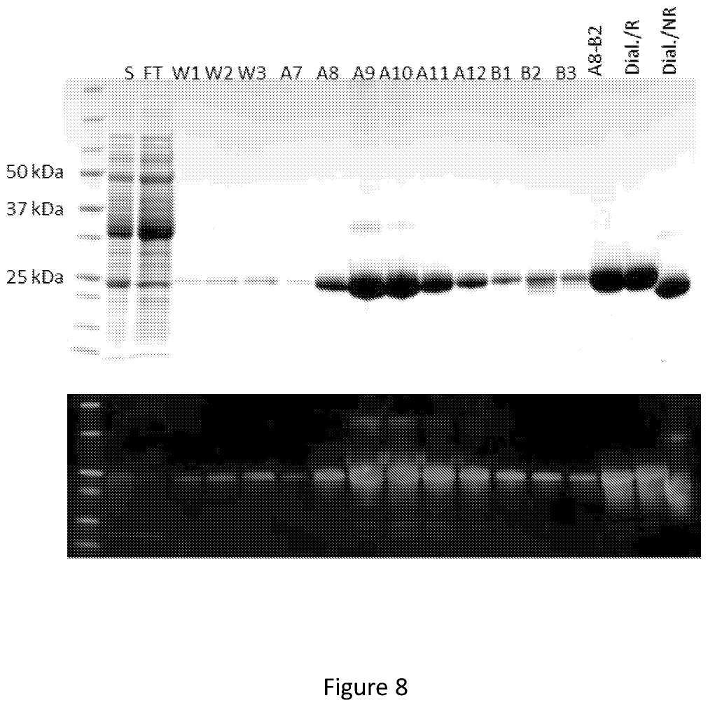

Large scale affinity purification of RBD-Delta from M5517 fermentation is shown as an example in FIG. 8 for stained SDS-PAGE and Western analysis. Detection in the Western is the same as used in the screening of transformants. Results show that the purification of RBD-Delta variant is quite similar to Wuhan-RBD-C-tag and resulted in purified protein of similar purity level as Wuhan-RBD-C-tag. Large scale affinity purification of RBD-Delta-C-tag from M5577 fermentation resulted in purified protein of similar purity level as Wuhan-RBD-C-tag (data not shown).

### Example 6: Co-Expression of Three SARS-CoV-2 RBD Variants Together in Protease Deficient C1 Strain

Three variants of Receptor Binding Domain (RBD) of SARS-CoV-2 spike protein were co-expressed in the protease deficient C1 strain DNL155. The three variants are: 1) RBD_B.1.1.7-UK (alpha-UK) having N501Y mutation, 2) RBD_B.1.351-SA (beta-SA) having K417N, E484K and N501Y mutations and 3) RBD_1.1.28.1(P.1)-BR (gamma-BR) having K417T, E484K and N501Y mutations. Same sequences for the variants were used as in single variant expression C1 strains M5260, M5266 and M5270 in Example 4. Each of the three variants were expressed from a single expression cassette. Alpha-UK and beta-SA variants were integrated into the bgl8 locus and gamma-BR into the cbh1 locus.

First, a gamma-BR expression plasmid targeting to cbh1 locus was constructed. The synthesized gamma-BR fragment in a GenScript plasmid was cut out with MssI restriction enzyme and cloned by Gibson Assembly (NEBuilder® HiFi DNA Assembly Cloning Kit, New England Biolabs) method into the PacI site between C1 bgl8 promoter and C1 bgl8 terminator to the expression vector pMYT0570 harbouring a pyr4 marker gene. Plasmid of correct sequence was given the plasmid number pMYT1578 (SEQ ID NO: 30).

Strain construction for co-expression of three variants was performed in two transformation steps. pMYT1578 was transformed to the DNL156 strain from which fourteen protease genes have been deleted. DNL156 is the pyr4-version of DNL155, an auxotrophic strain unable to grow without uracil/uridine supplementation. When transformed with an expression cassette with a pyr4 marker DNL156 restores the ability to grow without uracil/uridine supplementation. Transformation was performed as for the M4169 strain in Example 1 and transformants were selected for the pyr4-positive phenotype. Screening of transformants by 24-well cultivation was performed as transformants producing as in Example 4. Transformants producing the gamma-BR-C-tag variant were purified by single colony plating followed by PCR and qPCR screening. 5′ integration of the expression cassette in the cbh1 locus was shown by a PCR reaction with the primers set forth as SEQ ID NOs: 52 (oMYT0744) and 42 (oMYT2823) and 3′ integration of the expression cassette in the bgl8 locus was shown by a PCR reaction with the primers set forth as SEQ ID NOs: 53 (oMYT0027) and 54 (oMYT129). The loss of cbh1 ORF was shown by a PCR reaction with the primers set forth as SEQ ID NOs: 55 (oMYT0116) and 56 (oMYT0117). The loss of cbh1 ORF was further verified by qPCR with the primers set forth as SEQ ID NOs: 57 (oMYT0485) and 58 (oMYT0486).

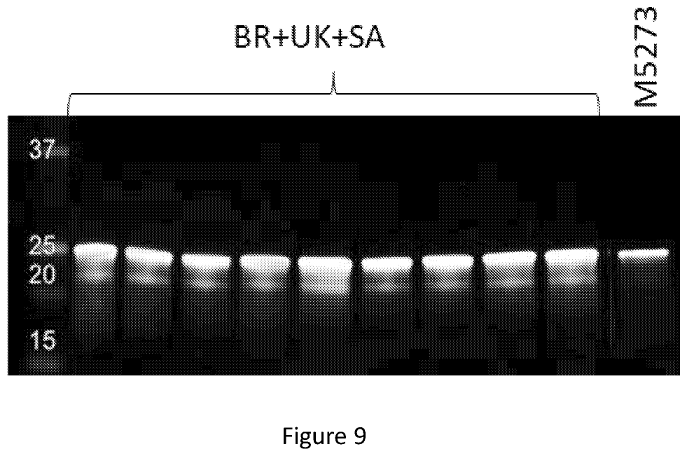

One verified transformant was at stored at −80° C. and given the strain number M5273. The M5273 strain was then transformed with alpha-UK 5′ arm expression plasmid pMYT172 and beta-SA 3′arm expression plasmid pMYT1575 as described in Example 4 introducing one copy of alpha-UK (in forward direction) and one copy of beta-SA (in reverse direction) into bgl8 locus. The transformation was performed as described for the M4169 strain in Example 1 and transformants were selected for nia1+ phenotype and hygromycin resistance. Screening of transformants by 24-well plate cultivation was performed as for expression of individual RBD-C-tag variants in Example 4. FIG. 9 is an example of such Western blotting results. Strong signals of the expected size with both primary antibodies were detected and the level of RBD was increased in transformants harbouring all three variant expression cassettes as compared to the intermediate strain M5273 harbouring only the expression cassette of gamma-BR variant. Single colony plating was performed as for the M4169 strain in Example 1. The resulting clones were verified by PCR for correct integration to both bgl8 and in cbh1 loci as above and for the presence of all three variant genes. Purity of clones, meaning the complete loss of bgl8 and cbhi1 was verified by qPCR as above. Single colony clones were verified by PCR from correct integration of gamma-BR in cbh1 locus as above and the 5′integration reaction verified also the presence of gamma-BR gene. The correct integration of alpha-UK and beta-SA in bgl8 locus was verified by PCR as in Example 4 and primer pair is designed so that the same integration PCR reactions also verify the presence of alpha-UK and beta-SA genes. Purity of clones for the complete loss of cbhi1 was verified by qPCR as above and for the complete loss of bgl8 by qPCR as in Example 1. One verified transformant was stored at −80° C. and given the strain number M5407.

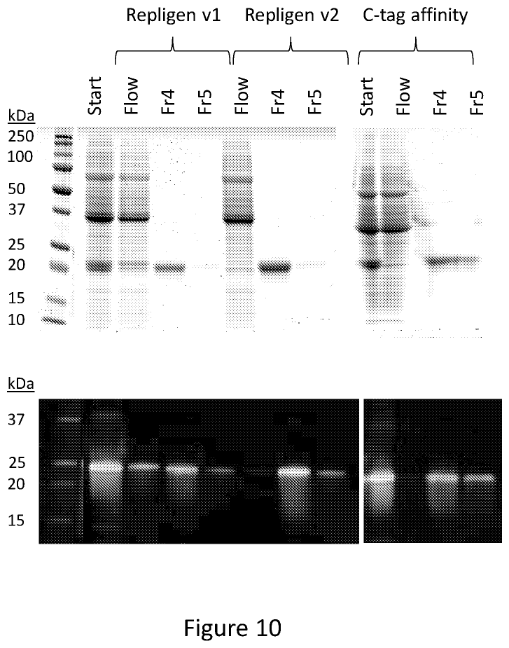

The C1 M5407 strain was cultivated in three parallel 1 L bioreactors in a fed-batch process in a medium with yeast extract as an organic nitrogen source and glucose as a carbon source. The cultures were performed at 38° C. for five days. Variant RBD-C-tag mix was purified in small scale from all bioreactor samples with three affinity resins; 1) C-tag affinity, 2) Repligen v1 and 3) Repligen v1 as described in Examples 1 and 4. FIG. 10 shows an example of Western blotting of purifications with all three resins, detection is with same antibodies as in Example 4 with individual RBD variants. Purified samples were analysed by Intact Mass Liquid Chromatography-Mass Spectrometry (LC-MS) analysis to determine the ratio of each variant in samples. Samples were first treated with PngaseF to remove glycans followed by intact mass reverse phase UPLC-MS. Table 1 below shows the proportions of the variants in samples purified by C-affinity and by Repligen resin v2. The ratio of the variants is consistent between the parallel fermentations. Additionally, the ratio is similar in samples purified with two different affinity resins. Samples purified by Repligen v1 contained over 90% of UK as v1 resin is not binding beta-SA or gamma-BR efficiently (data not shown).

TABLE 1

| | | C-tag | C-tag | | | |
|---|---|---|---|---|---|---|
| | C-tag affinity | affinity | affinity | Repligen v2 | Repligen v2 | Repligen v2 |
| Fermentation | alpha-UK | beta-SA | gamma-BR | alpha-UK | beta-SA | gamma-BR |
| MT508 | 49.0% | 15.7% | 35.3% | 49.2% | 15.6% | 35.2% |
| MT533 | 47.8% | 15.7% | 36.4% | 45.9% | 15.8% | 38.4% |
| MT534 | 46.9% | 15.1% | 38.0% | 45.1% | 15.2% | 39.8% |

The yield after a purification step was: BR—0.6 g/L, SA—0.29 g/L, UK—1.05 g/L.

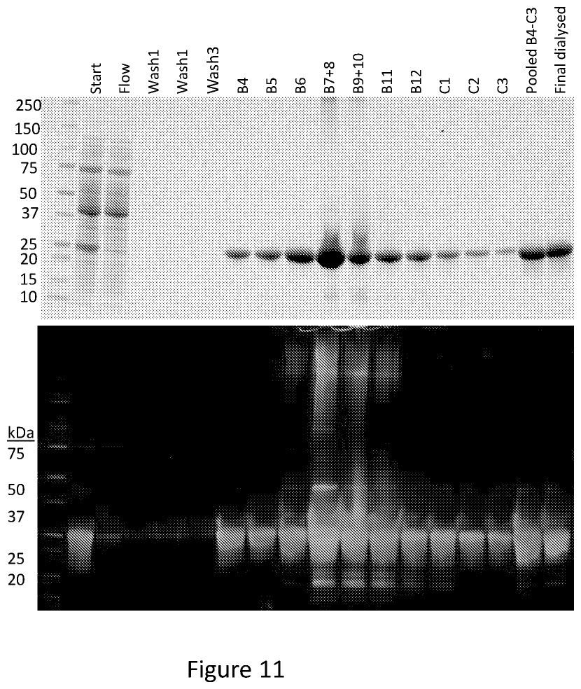

Variant RBD-C-tag mix was also purified in larger scale with Repligen v2 resin as described in Example 4. FIG. 11 is shown as an example of larger scale purifications of variant-RBD-C-tag mix by Repligen v2 resin. Intact LC-MS analysis was performed in similar manner as for the small-scale purification samples above and ratio of variants was alpha-UK 49.4%, beta-SA 17.5% and gamma-BR 33.1%.

### Example 7: ACE2-Binding ELISA Assay of C1 Produced RBD Proteins

The binding activity of C1 produced SARS-CoV-2 RBD proteins to human Angiotensin Converting Enzyme-2 (ACE2) was studied in Enzyme-Linked Immunosorbent Assay (ELISA). The protocol was as follows; the microtiter ELISA plate was coated with recombinant human ACE2 receptor (SinoBiological). RBD-C-tag proteins purified by Repligen v1 or v2 resin were diluted and incubated in the ACE2-coated wells, where the immobilized ACE2 bind the SARS-CoV2 RBD proteins. The bound RBD was detected by the same Capture Select Biotin Anti-C-tag conjugate (ThermoFisher) that was used in Western blotting, the secondary detection agent was Streptavidin-HRP (Cytiva). 3,3′,5,5′-tetramethylbenzidine (TMB) substrate, which is reactive with Horseradish Peroxidase, was added and produced colour in a colorimetric reaction. The amount of substrate reacted is proportional to the concentration of the RBD protein present in the wells. The enzymatic reaction was stopped with sulfuric acid, absorbance at 450 nm was measured and results were analysed with 4-parameter logistic (4PL) analysis. A C1 produced Wuhan RBD-C-tag reference, purified for toxicology studies (BTG, Israel), was used in each assay as a control.

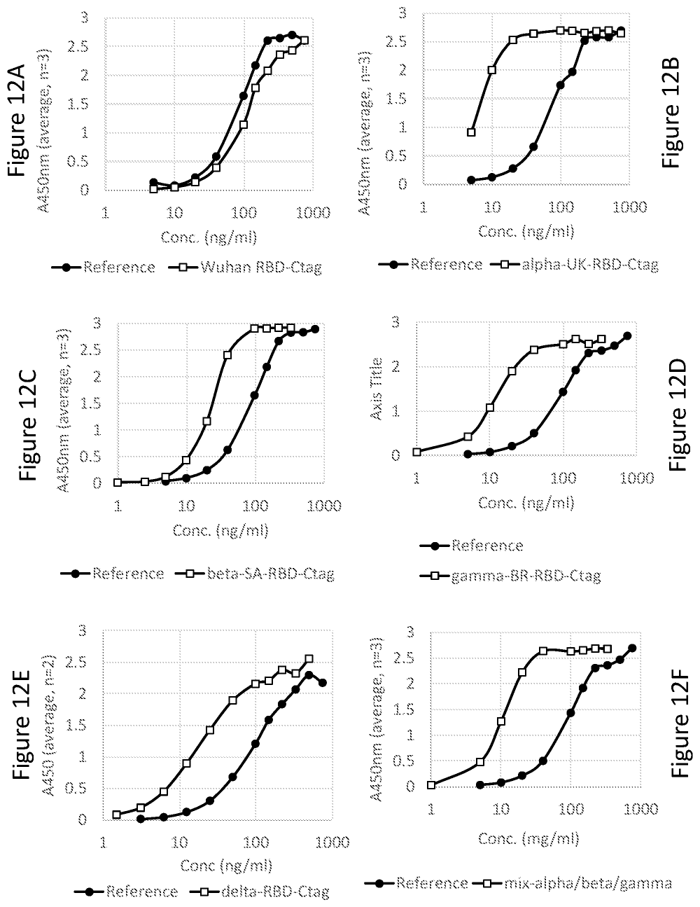

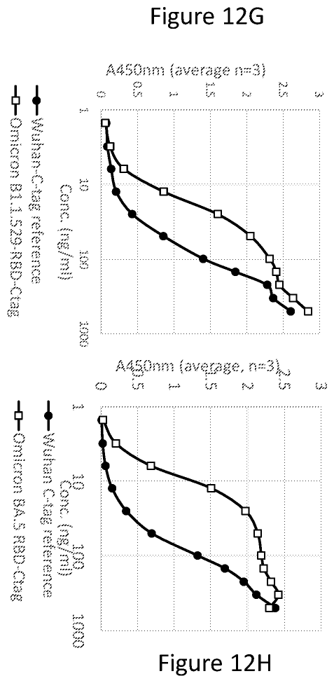

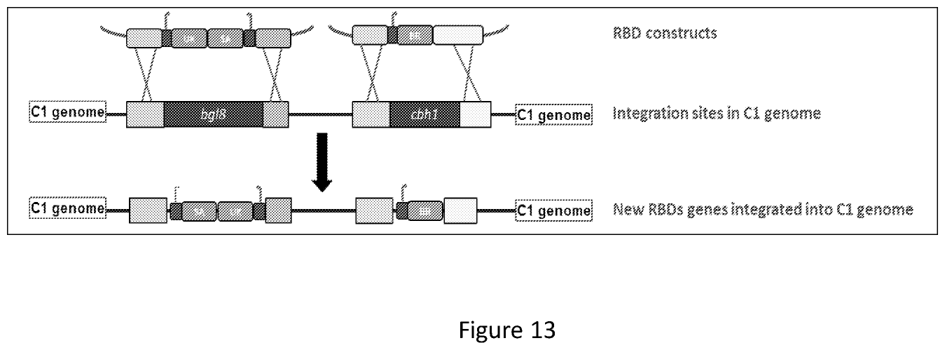

All the RBD molecules produced in C1 showed ACE2 binding activity in the ELISA assay. FIG. 12 shows examples of the results of ACE2-ELISA binding assays. Samples were analysed in triplicate and average values are shown in the graphs. The Wuhan-RBD-C-tag described herein had slightly lower activity as compared with the reference which is also Wuhan-RBD-C-tag. This is likely due to the fact that the reference is purified by other methods and is more polished than Wuhan RBD-C-tag after a single-step affinity purification. All the purified variant RBD-C-tag proteins were more active than the reference. Alpha-UK had over 11-fold, beta-SA about 3-fold, gamma-BR about 7.5-fold, delta about 4.5-fold, omicron B.1.1.529 about 4-fold and omicron BA.5 about 8-fold higher binding activity in the ELISA assay. The mix of three variants (alpha-UK/beta-SA/gamma-BR) had nearly 9-fold higher activity than the reference.

### Example 8: Generation of Variant RBD-C-Tag Mix by Co-Cultivation of Three C1 Strains Each Producing a Variant of RBD-C-Tag

An alternative approach for the co-expression of several RBD variants in a same C1 strain to generate a RBD variant mix is to perform mixed cultivations of C1 strains each expressing a RBD variant. This approach was executed as follows: Three C1 strains; M5260 (alpha-UK), M5266 (beta-SA) and M5577 (delta-C-tag) were cultivated together in ambr250 bioreactors in a fed-batch process in a medium with yeast extract as an organic nitrogen source and glucose as a carbon source at 38° C. for five days. Strains were first pre-cultivated as separate cultivations, then mixed and the mixed preculture was used to inoculate two parallel ambr250 bioreactors. Small-scale protein purification of the RBD-C-tag mixes (day three and day five samples) was performed by C-tag affinity as in Example 1 and purified RBD-C-tag variant mixes were analysed by intact LC-MS analysis as described in Example 6. Table 2 below shows the proportions of the variants in samples. Results indicate quite good reproducibility in the proportions between the parallel cultures. Though the ratio is not exact 1:1:1, all three RBD variants are present in adequate proportion. Notably, proportion of the RBD-C-tag variants may change during cultivations as seen in this example cultivation. Proportion of Alpha-UK variant is reducing during cultivation from day three to day five whereas the proportion of beta-SA variant is increasing during the same time span.

TABLE 2

| | alpha-UK | beta-SA | delta |
|---|---|---|---|
| Fermentation | Day3 → Day5 | Day3 → Day5 | Day3 → Day5 |
| MTA324 | 45.6% → 28.2% | 29.7% → 50.9% | 24.7% → 20.9% |
| MTA325 | 38.4% → 29.5% | 33.9% → 48.5% | 27.8% → 22.0% |

### Example 9: Expression of Two SARS-CoV-2 RBD Omicron Variants Separately in Protease Deficient C1 Strain

Two omicron variants of Receptor Binding Domain (RBD) of SARS-CoV-2 spike protein were expressed in the protease deficient C1 strain DNL155. The two omicron variants are: 1) RBD-omicron B.1.1.529 amino acid sequence is set forth in SEQ ID NO: 59 (including C1 cbh1 signal sequence, GSG-linker and C-tag), and DNA sequence in SEQ ID NO: 60; 2) RBD-omicron BA.5, amino acid sequence is set forth in SEQ ID NO: 63 (including C1 cbh1 signal sequence, GSG-linker and C-tag), and DNA sequence in SEQ ID NO: 64; Both omicron variants contained the residues 333-527 of the Spike protein from SARS-CoV-2, a Gly-Ser-linker and the C-tag. The fragment of both variants was synthesized by GenScript (USA). The synthetized fragment design was similar to the Wuhan RBD with C-tag as in Example 1 except that the Gly/Ser-linker between the RBD variant and the C-tag was three amino acids long whereas in Wuhan RBD-C-tag the linker was five amino acids long.

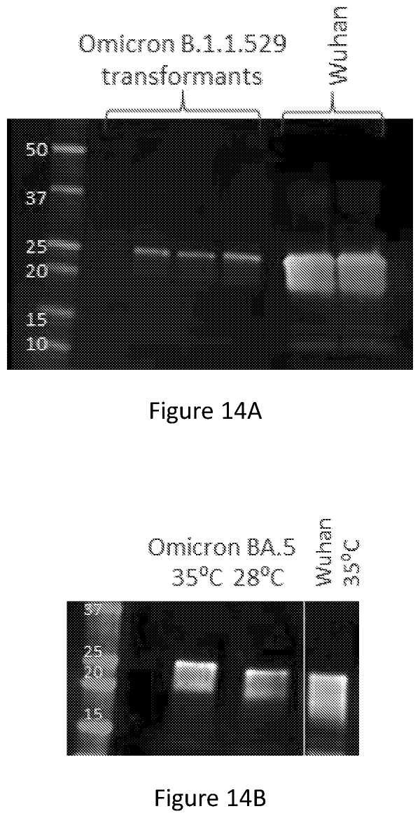

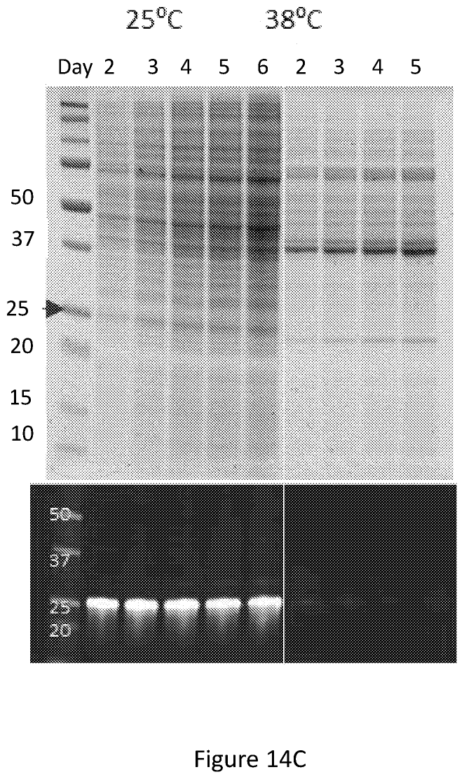

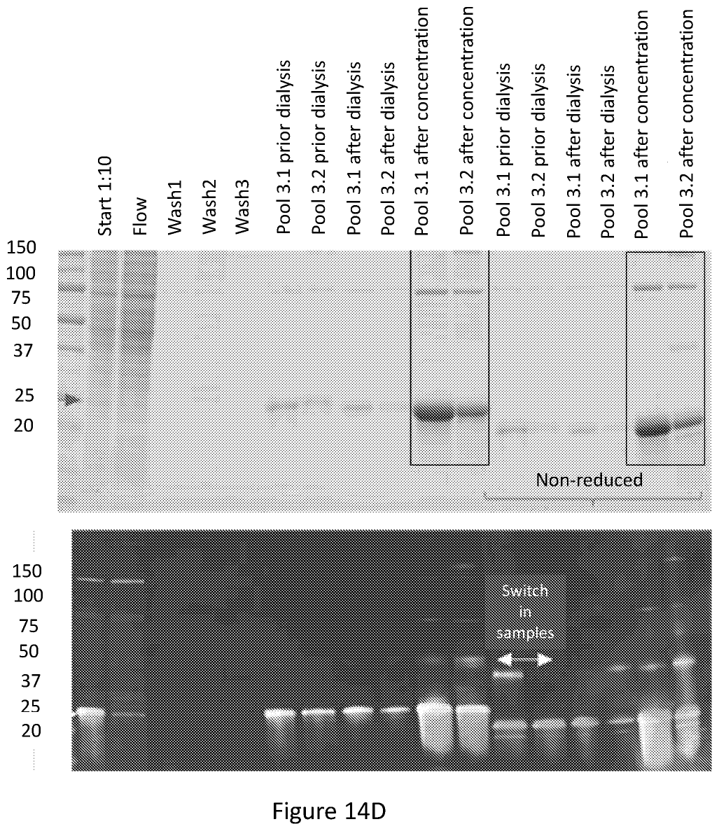

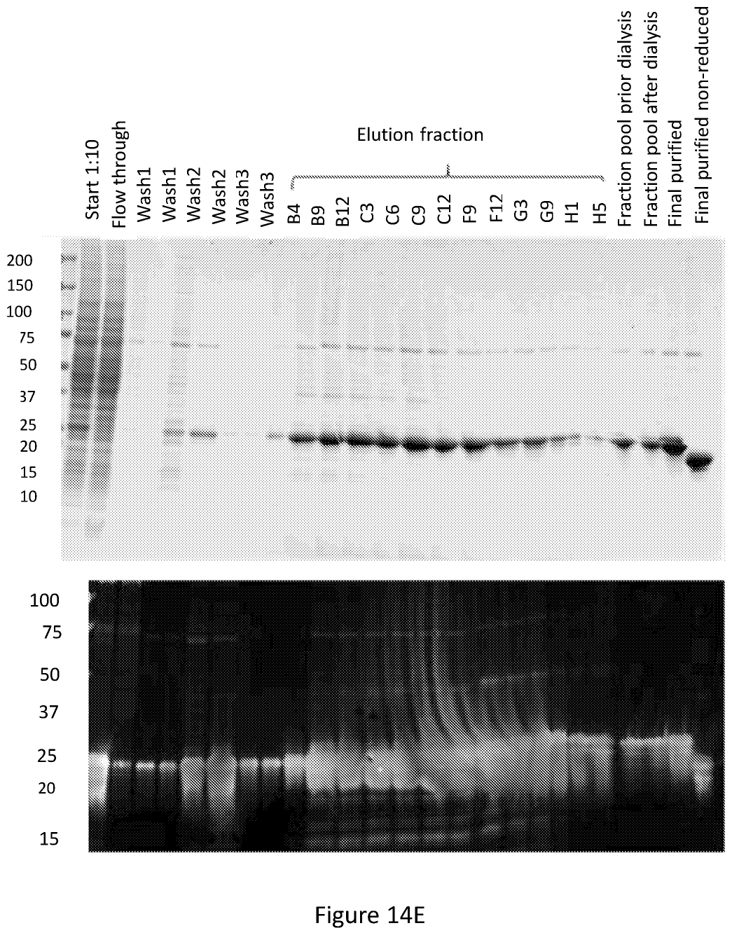

RBD-Omicron B.1.1.529 was expressed as: 1) having one expression cassette and 2) having two expression cassettes in the same locus. Two plasmids (5′arm and 3′arm), both harbouring one expression cassette, were constructed. For the 5′arm plasmid, synthesized fragment in GenScript plasmid was cut out with MssI restriction enzyme and cloned by Gibson Assembly (NEBuilder® HiFi DNA Assembly Cloning Kit, New England Biolabs) method into the PacI site of the C1 expression vector pMYT1055 between endogenous C1 bgl8 promoter and C1 chi1 terminator. Plasmid of correct sequence was given the plasmid number pMYT1822 (SEQ ID NO: 61). For the 3′arm plasmid, synthesized fragment was amplified by PCR from the GenScript plasmid and cloned by Gibson Assembly (NEBuilder® HiFi DNA Assembly Cloning Kit, New England Biolabs) method into the PacI site of the C1 expression vector pMYT1056 between endogenous C1 bgl8 promoter and C1 bgl8 terminator. Plasmid of correct sequence was given the plasmid number pMYT1823 (SEQ ID NO: 62). To generate the strain having one expression cassette, the expression vector pMYT1822 and a mock vector pMYT1140 (SEQ ID NO: 13), which is needed for completion of the hygromycin resistance marker gene and integration to the bgl8 locus, were digested with MssI and transformed to the DNL155 strain from which fourteen protease genes have been deleted in similar manner as in the Example 1. To generate the strain having two expression cassettes, pMYT1822 (5′arm expression plasmid) and pMYT1823 (3′arm expression plasmid) plasmids were digested with MssI and co-transformed to the DNL155 strain from which fourteen protease genes have been deleted as in Example 1. Similar to other RBD variants strains of two copies, the orientation in the genomic locus is such that one gene copy is in forward direction while the second gene copy is in reverse direction. The transformation and screening of transformants by 24-well cultivation was performed as in Example 1 except that culture supernatants were analysed by Western blotting as in Example 4. Western analysis (FIG. 14A) showed a weak signal of the expected size in a number transformants, indicating omicron B.1.1529 being produced at lower level than Wuhan-RBD-C-tag. Producing transformants of both one or two expression cassettes were purified by single colony plating, and purified clones were verified by PCR for correct integration of the expression cassette and by qPCR for clone purity as in Example 1 except the 5′ integration of the expression cassette in the bgl8 locus was shown by a PCR reaction with the primers set forth as SEQ ID NOs: 40 (oMYT0606) and 66 (oMYT2749). For the strain of two expression cassette, presence of two RBD genes were verified by PCR as in Example 2. qPCR results showed that the transformants analysed by qPCR were pure prior to single colony plating purification. One verified transformant of each strain was stored at −80° C. and given the strain number M5890 for the strain of one expression cassette and the strain number M5892 for the strain of two expression cassettes.

RBD-Omicron B.A.5 was expressed only as having one expression cassette. Synthesized fragment in GenScript plasmid were cut out with MssI restriction enzyme and cloned by Gibson Assembly (NEBuilder® HiFi DNA Assembly Cloning Kit, New England Biolabs) method into the PacI site of the C1 expression vector pMYT1055 between endogenous C1 bgl8 promoter and C1 chi1 terminator. Plasmid of correct sequence was given the plasmid number pMYT1990 (SEQ ID NO: 65). The expression vector pMYT1990 and a mock vector pMYT1140 (SEQ ID NO: 13), which is needed for completion of the hygromycin resistance marker gene and integration to the bgl8 locus, were digested with MssI and transformed to the DNL155 strain from which fourteen protease genes have been deleted in similar manner as in the Example 1. The transformation and screening of transformants by 24-well cultivation was performed as in Example 1 except 24-well cultivations were performed both at 28° C. and in 35° C. Culture supernatants were analysed by Western blotting as in Example 4. Western analysis (FIG. 14B, example of one positive transformant) showed a quite strong signal of the expected size in some transformants at both temperatures, indicating that omicron BA.5 was produced at somewhat higher level than omicron B.1.1529. Producing transformants were purified by single colony plating, and purified clones were verified by PCR for correct integration of the expression cassette and by qPCR for clone purity as above for RBD-omicron B.1.1.529 except no PCR verification of the second expression cassette. One verified transformant was stored at −80° C. and given the strain number M6369.

M5890 (one expression cassette) and M5892 (two expression cassettes) strains of omicron B.1.1529 variant and M6369 of omicron BA.5 variant were cultivated in 1 L bioreactors in a fed-batch process as described in Example 1. M5890 and M5892 strains were first cultivated at 38° C. As no product was observed in stained SDS-PAGE nor in Western blotting, the M5890 strain was recultivated in 1 L bioreactors in a fed-batch process in which the temperature was reduced from 38° C. to 25° C. in the beginning of the feeding phase and cultivated at 25° C. all feeding phase. Reducing temperature improved the productivity significantly (FIG. 14C). M6369 strain of omicron BA.5 was cultivated only at 25° C. as the M5890 strain above and production was obtained. Both omicron variants were purified by C-tag affinity in small scale and in large scale as in Example 1 except in large scale purification, wash and elution steps were performed as follows: after sample loading, the column was first washed with 5CV 1×PBS, second wash with 5CV 1×PBS with additional 0.5M NaCl and third wash with 5CV 1×PBS. Elution was performed first as linear gradient elution of 8CV from 0% to 70% elution buffer followed with 2CV 100% elution buffer. After dialysis, purified proteins were concentrated with centrifugal concentrators. The resulted purified proteins were estimated as 80-85% pure (FIGS. 14D and 14E).

### Example 10: Generation of Antibody Producing Strains with CRISPR-Cas9 Technology

Protease deficient C1 strain M5824 was transformed with a construct expressing a monoclonal antibody using the CRISPR-Cas9 technology. The construct did not contain specific sequences homologous with the targeted loci. Difference between the CRISPR-Cas9 technology and random integration is the presence of guide RNAs designed to guide the Cas9 enzyme to specific loci where the enzyme makes double stranded breaks to the DNA removing a piece from the targeted gene which is then replaced by the expression construct. crRNAs were designed to guide the Cas9 enzyme to cellobiohydrolase (cbh1), β-glucosidase (bgl8), cellobiose dehydrogenase (cbd), chitinase (chi1), a glycoside hydrolase family 6 gene (GH6), a glycoside hydrolase family 61 gene (GH61) and a carbohydrate-binding WSC gene loci of C1. Two crRNAs were designed for each locus. RNA sequences of the crRNAs are set forth in SEQ ID NO: 67-80. Sequences include a 20 nt target-specific protospacer region and a 16 nt tracrRNA fusion domain. The CRISPR components including crRNA:tracrRNA duplex and Cas9 enzyme were added to the C1 transformation as preassembled ribonucleoproteins. Transformants were streaked onto selective medium plates and inoculated from the streaks to liquid cultures in 96-well plates. The medium components were as in Example 1. The 96-well plates were incubated at 35° C. with 800 RPM shaking for four days. Culture supernatants were collected and analysed by dot blots. Transformants showing the highest signal intensities according to the dot blot were single-colony purified and cultivated in bioreactors in a fed-batch process in a medium with yeast extract as an organic nitrogen source and glucose as a carbon source. The cultures were performed at 38° C. for seven days. Produced antibody was purified and quantified using an affinity chromatography method with ÄKTA Start protein purification system (Cytiva). The quantity of the purified antibody was calculated by integrating the UV trace of the elution peak with the Unicorn 1.0 software included in the ÄKTA Start system. Table 3 shows production levels of the best C1 strains obtained with CRISPR-Cas9 technology. In a strain with similar background where one copy of the antibody expression cassette was integrated to one specific locus with targeted integration the production level of the same antibody was about 2.5 g/(about 0.048 g/g dry weight)

TABLE 3

| | Production level, | Production level, |
|---|---|---|
| Strain | g/l, day 7 | g/g dry weight, day 7 |
| M5824-1 | 22.3 | 0.47 |
| M5824-2 | 16.1 | 0.53 |
| M5824-3 | 15.4 | 0.50 |

Sequences

| SEQ ID | |
|---|---|
| NO: # | Description |
| 1 | rDNA of Thermothelomyces heterothallica C1 |
| 2 | RBD_B.1.1.7-UK-amino acids |
| 3 | RBD_B.1.1.7-UK-nucleic acid |
| 4 | RBD_B.1.351-SA-amino acids |
| 5 | RBD_B.1.351-SA-nucleic acids |
| 6 | RBD_1.1.28.1(P.1)-BR-amino acids |
| 7 | RBD_1.1.28.1(P.1)-BR-nucleic acids |
| 8 | Amino acid sequence of Kex2 (Alp11) |
| | (jgi\|Spoth2\|2293998\|fgenesh_kz1_kg.chromosome_1_#_65_#_365) |
| 9 | Amino acid sequence of Alp7 |
| | (jgi\|Spoth2\|2309026\|fgenesh_kz1_kg.chromosome_5_#_1195_#_103253 |
| 10 | Amino acid sequence of Wuhan RBD-C-tag, including C1 cbh1 signal |
| | sequence (MYAKFATLAALVAGAAA), GGGGS-linker and C-tag |
| | (EPEA) |
| 11 | DNA sequence of Wuhan RBD-C-tag, including C1 cbh1 signal sequence, |
| | GGGGS-linker and C-tag |
| 12 | Full nucleotide sequence of plasmid pMYT1142 |
| 13 | Full nucleotide sequence of plasmid pMYT1140 |
| 14 | Full nucleotide sequence of plasmid pMYT1366 |
| 15 | Amino acid sequence of Wuhan RBD no tag, including C1 cbh1 signal |
| | sequence (MYAKFATLAALVAGAAA) |
| 16 | DNA sequence of Wuhan RBD-C-tag, including C1 cbh1 signal sequence |
| 17 | Full nucleotide sequence of plasmid pMYT1237 |
| 18 | Amino acid sequence of alpha-UK-RBD-C-tag, including C1 cbh1 signal |
| | sequence (MYAKFATLAALVAGAAA (SEQ ID NO: 81)), GSG-linker |
| | and C-tag (EPEA) |
| 19 | DNA sequence of alpha-UK-RBD-C-tag, including C1 cbh1 signal |
| | sequence, GSG-linker and C-tag |
| 20 | Full nucleotide sequence of plasmid pMYT1572 |
| 21 | Full nucleotide sequence of plasmid pMYT1573 |
| 22 | Amino acid sequence of beta-SA-RBD-C-tag, including C1 cbh1 signal |
| | sequence (MYAKFATLAALVAGAAA (SEQ ID NO: 81)), GSG-linker |
| | and C-tag (EPEA) |
| 23 | DNA sequence of beta-SA-RBD-C-tag, including C1 cbh1 signal sequence, |
| | GSG-linker and C-tag |
| 24 | Full nucleotide sequence of plasmid pMYT1574 |
| 25 | Full nucleotide sequence of plasmid pMYT1575 |
| 26 | Amino acid sequence of gamma-BR-RBD-C-tag, including C1 cbh1 signal |
| | sequence (MYAKFATLAALVAGAAA (SEQ ID NO: 81)), GSG-linker |
| | and C-tag (EPEA) |
| 27 | DNA sequence of gamma-BR-RBD-C-tag, including C1 cbh1 signal |
| | sequence, GSG-linker and C-tag |
| 28 | Full nucleotide sequence of plasmid pMYT1576 |
| 29 | Full nucleotide sequence of plasmid pMYT1577 |
| 30 | Full nucleotide sequence of plasmid pMYT1578 |
| 31 | Amino acid sequence of RBD-Delta, including C1 cbh1 signal sequence |
| | (MYAKFATLAALVAGAAA (SEQ ID NO: 81)) |
| 32 | DNA sequence of RBD-Delta, including C1 cbh1 signal sequence |
| 33 | Amino acid sequence of RBD-Delta-C-tag, including C1 cbh1 signal |
| | sequence (MYAKFATLAALVAGAAA (SEQ ID NO: 81)), GSG-linker |
| | and C-tag (EPEA) |
| 34 | DNA sequence of RBD-Delta-C-tag, including C1 cbh1 signal sequence, |
| | GSG-linker and C-tag |
| 35 | Full nucleotide sequence of plasmid pMYT1717 (RBD-Delta, no C-tag) |
| 36 | Full nucleotide sequence of plasmid pMYT1716 (RBD-Delta-C-tag) |
| 37 | Amino acid sequence of RBD-beta-SA, including C1 cbh1 signal sequence |
| | (MYAKFATLAALVAGAAA (SEQ ID NO: 81)) |
| 38 | DNA sequence of RBD-beta-SA, including C1 cbh1 signal sequence |
| 39 | Full nucleotide sequence of plasmid pMYT1715 (RBD-beta-SA) |
| 59 | Amino acid sequence of RBD-omicron B.1.1.529, including C1 cbh1 signal |
| | sequence (MYAKFATLAALVAGAAA (SEQ ID NO: 81)) |
| 60 | DNA sequence of RBD-omicron B.1.1.529, including C1 cbh1 signal |
| | sequence |
| 61 | Full nucleotide sequence of plasmid pMYT1822 (RBD-omicron B.1.1.529) |
| 62 | Full nucleotide sequence of plasmid pMYT1823 (RBD-omicron B.1.1.529) |
| 63 | Amino acid sequence of RBD-omicron B.A.5, including C1 cbh1 signal |
| | sequence (MYAKFATLAALVAGAAA (SEQ ID NO: 81)) |
| 64 | DNA sequence of RBD-omicron B.A.5, including C1 cbh1 signal sequence |
| 65 | Full nucleotide sequence of plasmid pMYT1990 (RBD-omicron B.A.5) |
| 81 | cbh1 signal sequence |

Primer sequences:

| SEQ ID NO: | Name | Primer sequence |
|---|---|---|
| 40 | oMYT0746 | CTCGCACCGTAAGGACCAAG |
| 41 | oMYT0606 | CCACGTAAGCGGAAGTTG |
| 42 | oMYT2823 | CGTTGAAGACCTCGCCGAA |
| 43 | oMYT1277 | AGCGTGACCCACTCAGGTAA |
| 44 | oMYT0748 | AGCGTGACCCACTCAGGTAA |
| 45 | oMYT0529 | CACTACGTCAAGTTCGTCGA |
| 46 | oMYT0532 | GCCGTAGAAGTCGTTGGAG |
| 47 | oMYT0649 | CGGTCACGTAGATCTTGGG |
| 48 | oMYT0648 | AACCTCGAGACGCTCTTCTA |
| 49 | oMYT0086 | CATCATCCGTCTTCCATCC |
| 50 | oMYT0107 | GGTAAACATGTCCAGGCTTC |
| 51 | oMYT0089 | CTTCAGAGACAGGAGCCGT |
| 52 | oMYT0744 | AGGAAGGAGACATCGCTCTTTT |
| 53 | oMYT0027 | AGCCTGGAAGGCCTATCTGG |
| 54 | oMYT0127 | TAGCGCGAATACTGCTGTGG |
| 55 | oMYT0116 | CCTACACTTGCACCAAGCTG |
| 56 | oMYT0117 | GGCACGTATGGTCGTTTGTA |
| 57 | oMYT0485 | CCCTCTACTTCGTGTCCATG |
| 58 | oMYT0486 | CAGTTCTCTACGTTGGCCTC |
| 66 | oMYT2749 | AGCTTGTAGTTGTAGTCGGCGAT |

crRNA sequences:

| SEQ ID | | |
|---|---|---|
| NO: | Name | Sequence |
| 67 | bg18_crRNA_1 | CUACGGCAUGAACCACUACAGUUUUAGAGCUAUGCU |
| 68 | bg18_crRNA_2 | GGGUCCUUGAACUUACGAGGGUUUUAGAGCUAUGCU |
| 69 | cbd_crRNA_1 | CGACCCCAUGAAUCUCAGCGGUUUUAGAGCUAUGCU |
| 70 | cbd_crRNA_2 | CAUCUCCUCAGCAUUCACCGGUUUUAGAGCUAUGCU |
| 71 | cbh1_crRNA_1 | AAGCACUAUGAGCAAUGCGGGUUUUAGAGCUAUGCU |
| 72 | cbh1_crRNA_2 | GCACUUGGACCACGUCAGCGGUUUUAGAGCUAUGCU |
| 73 | chil_crRNA_1 | CUCACUGGCCAAUCGAACAGGUUUUAGAGCUAUGCU |
| 74 | chil_crRNA_2 | CGGCUGUGCUGGCCGCCGCGGUUUUAGAGCUAUGCU |
| 75 | GH61_crRNA_1 | CGCCCAUUAUACCCUCCCCAGUUUUAGAGCUAUGCU |
| 76 | GH61_crRNA_2 | UGAUGUUGAUAAGAAUGCCGGUUUUAGAGCUAUGCU |
| 77 | WSC_crRNA_1 | GUAGGUCUGCGCAAGGACGGGUUUUAGAGCUAUGCU |
| 78 | WSC_crRNA_2 | GGUGAGCGAGACUCAGUACGGUUUUAGAGCUAUGCU |
| 79 | GH6_crRNA_1 | GUACUCGUCGAACCACUGGCGUUUUAGAGCUAUGCU |
| 80 | GH6_crRNA 2 | CGUGGCGGCGACAAGACCACGUUUUAGAGCUAUGCU |

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A genetically modified filamentous fungus comprising at least one cell capable of producing at least two different immune-active molecules, said immune-active molecules are produced from at least two different transcription units, wherein the ratio between the amounts of the at least two immune-active molecules produced is from about 1:1 to about 1:10.

2. The genetically modified ascomycetous filamentous fungus of claim 1, wherein the immune-active molecules are antigens.

3. The genetically modified ascomycetous filamentous fungus of claim 2, wherein the antigens are viral antigens.

4. The genetically modified ascomycetous filamentous fungus of claim 3, wherein the viral antigens are selected from the group consisting of coronavirus, influenza virus, hepatitis B virus, hepatitis C virus, papillomavirus, HIV, HTLV-1, and EBV antigens.

5. The genetically modified ascomycetous filamentous fungus of claim 4, wherein the antigens are coronavirus antigens.

6. The genetically modified ascomycetous filamentous fungus of claim 5, wherein the antigens are coronavirus receptor binding domain (RBD), or a fragment thereof.

7-8. (canceled)

9. The genetically modified ascomycetous filamentous fungus of claim 1, wherein the fungal cell is capable of producing three immune-active molecules.

10. The genetically modified ascomycetous filamentous fungus of claim 9, wherein the three immune-active molecules are RBD antigens, or a fragment thereof, of three different SARS-COV-2 variants.

11. (canceled)

12. The genetically modified ascomycetous filamentous fungus of claim 1, wherein the immune-active molecules are antibodies or a fragment thereof.

13. (canceled)

14. The genetically modified ascomycetous filamentous fungus of claim 1, wherein the modified filamentous fungus comprises at least one cell having reduced expression and/or activity of at least one protease.

15-17. (canceled)

18. The genetically modified ascomycetous filamentous fungus of claim 1, wherein the ascomycetous filamentous fungus is of a genus within Pezizomycotina.

19-20. (canceled)

21. The genetically modified ascomycetous filamentous fungus of claim 1, said ascomycetous filamentous fungus is a Thermothelomyces heterothallica strain comprising rDNA sequence having at least 95%, or at least 96%, or at least 97%, or at least 98%, or at least 99% or 100% identity to the nucleic acid sequence set forth in SEQ ID NO: 1.

22-24. (canceled)

25. The genetically modified ascomycetous filamentous fungus of claim 1, wherein at least one of the immune-active molecules comprises a signal peptide for secretion.

26. The genetically modified ascomycetous filamentous fungus of claim 1, wherein the genetically modified ascomycetous filamentous fungus is capable of producing at least 0.1 gr of each viral antigen to a batch of Liter fermentation.

27. A method for producing a fungus capable of producing at least two immune-active molecules, the method comprising transforming at least one cell of the fungus with at least two exogenous polynucleotides, wherein each polynucleotide comprises a sequence encoding for an immune-active molecule.

28. (canceled)

29. A method of producing at least two immune-active molecules, the method comprising culturing the genetically modified fungus of claim 1 in a suitable medium; and recovering the produced immune-active molecules.

30. (canceled)

31. A combination of at least two immune-active molecules produced by a method according to the method of claim 26.

32. (canceled)

33. A cultivation comprising a mix of at least two genetically modified filamentous fungi, each fungus comprising at least one cell capable of producing a viral antigen, and wherein each of the at least two fungi is capable of producing a different viral antigen.

34-37. (canceled)

38. The genetically modified ascomycetous filamentous fungus of claim 41, wherein the transcription units are inserted to two, three, four or more different loci (‘landing site’) within of the fungal genome.

39-40. (canceled)

41. The genetically modified ascomycetous filamentous fungus of claim 1, wherein each of the transcription units is inserted to at least one locus within the fungal genome and wherein the locus is selected from the group consisting of cellobiohydrolase (cbh1), β-glucosidase (bgl8), cellobiose dehydrogenase (cdh), chitinase (chi1), a glycoside hydrolase family 6 gene (GH6), a glycoside hydrolase family 61 gene (GH61), a carbohydrate-binding WSC, and any combination thereof.
